(19) United States
(12) Patent Application Publication
(10) Pub. No.: US 2025/0064859 A1
(43) Pub. Date: Feb. 27, 2025

(54) HPSC-DERIVED ARTICULAR CHONDROCYTE COMPOSITIONS, SYSTEMS AND METHODS OF USE THEREOF

(71) Applicants: The Children's Medical Center Corporation, Boston, MA (US); The General Hospital Corporation, Boston, MA (US)

(72) Inventors: April M. Craft, West Roxbury, MA (US); Steven Pregizer, Boston, MA (US); Jenna Galloway, Boston, MA (US)

(21) Appl. No.: 18/847,536

(22) PCT Filed: Mar. 16, 2023
(86) PCT No.: PCT/US2023/064533

§ 371 Date: Sep. 16, 2024

Related U.S. Application Data
(60) Provisional application No. 63/320,334, filed on Mar. 16, 2022.

(51) Int. Cl. A61K 35/32; C12N 5/077

(52) U.S. Cl. CPC A61K 35/32; C12N 5/0655; C12N 2501/115; C12N 2501/15; C12N 2501/155; C12N 2506/02

(57) ABSTRACT

Improved compositions and methods for generating chondrocytes and cartilage tissues from human pluripotent stem cells are provided. Methods include use of one or more of FGF agonist, cAMP agonist, and TGFβ agonist to induce chondrogenesis in monolayer culture. Articular cartilage tissues generated using the methods have zonal organization similar to native cartilage tissue including surface chondrocytes and intermediate zone chondrocytes, with increased extracellular matrix components consistent with native cartilage tissue. These biochemical and mechanical properties make the cartilage tissue particularly suited for tissue implants in vivo.

### CROSS-REFERENCE TO RELATED APPLICATIONS

This application claims the benefit of and priority to U.S. Provisional Application No. 63/320,334 filed Mar. 16, 2022, which is hereby incorporated by reference in its entirety.

### STATEMENT REGARDING FEDERALLY SPONSORED RESEARCH OR DEVELOPMENT

This invention was made with government support under Grant No. R01AR073821 awarded by the National Institutes of Health. The government has certain rights in the invention.

### FIELD OF THE INVENTION

This invention is generally in the field of chondrocytes and cartilage and particularly methods for producing articular chondrocytes and articular cartilage-like tissue from human pluripotent stem cells.

### BACKGROUND OF THE INVENTION

Degenerative joint disease, also known as osteoarthritis, is among the top five most costly medical conditions in the United States. One of the challenges of repairing the articular cartilage that lines our joints is that this tissue forms prenatally, and regeneration does not normally occur after birth.

Damage to the articular cartilage often leads to the development of joint degeneration, or osteoarthritis, which causes lifelong pain and restricts the ability of patients to lead normal lives. Current treatments for damaged or degenerating cartilage are inadequate, primarily focusing on pain management and joint replacement (Redman et al., 2005). Successfully repairing damaged areas of articular cartilage using human pluripotent stem cell-based therapies may provide an effective way of preventing or delaying the onset of joint degeneration and improving quality of life for patients. Current cell-based therapies approved for cartilage repair are limited to autologous chondrocyte implantation (ACI) and its modified form, matrix-induced autologous chondrocyte implantation (MACI). These procedures require an initial operation to collect small pieces of cartilage, from which chondrocytes can be isolated and expanded before the re-implantation of the cultured cells during a second procedure. ACI and MACI, while moderately successful, have a number of disadvantages such as the requirement for multiple surgical procedures, donor site morbidity, the low numbers of cells retrieved, and the de-differentiation of chondrocytes during expansion. In addition, the efficacy of such technologies has not surpassed that of the current standard of care, microfracture, which involves perforating the subchondral bone, releasing cells from the bone marrow cavity to stimulate repair within the defect (Steadman et al., 2001). All of these approaches result in fibrocartilage-like repair tissue that lacks important biochemical and biomechanical properties of cartilage (LaPrade et al., 2008), which can lead to graft delamination and failure.

Human pluripotent stem cells (hPSCs) may provide solutions to many of the problems currently faced in treating damaged articular cartilage. The use of self-renewing human embryonic stem cells (hESCs) provides an opportunity to develop an ‘off’ the shelf cell source and/or tissue implant for cartilage repair without the need for the multiple procedures currently required for cell-based therapies. Further, a cartilage defect can be repaired using tissue generated from induced pluripotent stem cells (hiPSCs) derived from a patient's own cells, thus reducing the potential for immune rejection (Staerk et al., 2010; Takahashi et al., 2007). Although rigorous safety studies are required before the routine clinical use of iPSCs, many approaches to treat human disease using these cells are under development. Regenerative-medicine-based biotechnology companies and academic institutions are developing clinical grade iPSCs, and the first clinical trial for iPSC-derived platelets was approved in Japan in 2018 (Akabayashi et al., 2019). Clinical trials are also ongoing to establish the potential for hESC-derived progenitor cells to treat a number of conditions including ischemic heart disease and age-related macular degeneration (Ilic et al., 2015). The progression of these studies clearly demonstrates the need, and also the potential, for hPSCs to be used in the clinic. Both hESC and hiPSCs can be differentiated into either an articular-like or a hypertrophic, growth plate-like chondrocyte phenotype using chemically defined and precisely controlled directed differentiation methods (Craft et al., 2013; Craft et al., 2015). This protocol provides the signaling embryonic cells experience during cartilage development in utero and involves the induction of a primitive-streak-like mesoderm followed by specification of the chondrogenic mesoderm and finally the generation of chondrocyte progenitors and cartilage tissues. Long term exposure to transforming growth factor β3 (TGFβ1, TGFβ2, and TGFβ3) further induces the formation of an articular cartilage-like tissue, marked by the expression of SOX9, COL2A1 and PRG4, the latter encoding the proteoglycan lubricin, which is important for maintaining a frictionless articular surface. Conversely, the exposure of chondrocyte progenitors to bone morphogenetic protein 4 (BMP4) induces the expression of hypertrophic chondrocyte genes including COL10A1 and ALPL. When implanted subcutaneously into a mouse, TGFβ3-treated chondrocytes produced articular-cartilage-like matrix that resisted vascularization and ossification over a period of 12 weeks in vivo, while tissue produced by the hypertrophic BMP4-treated chondrocytes created a cartilaginous matrix that initiated remodeling and ossification (Craft et al., 2015).

In addition to hPSCs, other cellular sources are being investigated for use in cell-based therapies, in particular human mesenchymal stem cells (hMSCs). Human MSCs can be isolated from a number of post-natal tissues including adipose, synovial membrane, periosteum and bone marrow and are also capable of generating cartilage-like tissue when stimulated with TGFβ (De Bari et al., 2001a; De Bari et al., 2001b; Johnstone et al., 1998; Pittenger et al., 1999; Zuk et al., 2002). However, unlike hESCs and hiPSCs, hMSCs demonstrate high levels of donor-to-donor variability (Stoddart et al., 2012) and are not capable of maintaining a stable articular-cartilagelike phenotype in vivo. Instead, these cells progress towards hypertrophy in response to chondrogenic induction (Johnstone et al., 1998; Mueller et al., 2013). The relative homogeneity and phenotypic stability of hPSC-derived chondrocytes compared to hMSCs (Craft et al., 2015), in conjunction with their unlimited capacity for proliferation makes them an excellent candidate cell type for future cell-based therapies and studies of cartilage repair, as a prelude to repairing damaged tissue in patients.

There is still a need for methods to generate chondrocytes from hPSC, which reduce the number of steps and/or the duration needed, and which provide chondrocytes with a reduced tendency to de-differentiated following implantation.

It is therefore an object of the present invention to provide methods and reagents to produce cells forming articular cartilage.

It is a further object of the present invention to provide methods and reagents to produce cells which can proliferate in cell culture to form articular cartilage.

It is still an object of the present invention to provide methods of treating a subject in need thereof using chemically induced chondrocytes.

It is a further object of the present invention to provide methods of expanding and preserving chemically induced chondrocytes.

### SUMMARY OF THE INVENTION

Chemically induced chondrocytes or cartilage tissues engineered using chemically induced chondrocytes and extracellular matrix are disclosed. Cell culture media and methods for inducing chondrocytes from human pluripotent stem cells (hPSC) are also disclosed. Typically, the cell culture media is used in a four-stage process to induce chondrocytes from hPSC. The stages are:
Stage I, inducing the formation of primitive streak mesoderm from hPSC;
Stage II, inducing the formation of paraxial mesoderm;
Stage III, inducing conversion of paraxial mesoderm cells into chondrocytes; and
Stage IV, inducing chondrocytes to form mature cartilage tissues.

In some embodiments, the cell culture media useful for Stage I are supplemented with effective amounts of combinations selected from: an FGF agonist for example, FGF; a BMP4 agonist, for example BMP4; and a TGFβ agonist, for example Activin A; in an effect amount to induce induction of iPSC cells to form primitive streak mesoderm. In some embodiments, Stage I includes the formation of embryoid bodies. In other embodiments, Stage I does not include the formation of embryoid bodies. The cell culture media are optionally supplemented with a Wnt agonist.

In some embodiments, the cell culture media useful for Stage II are supplemented with effective amounts of small molecule and protein combinations selected from: a BMP4 inhibitor and/or an FGF agonist, for example FGF, in effective amounts to induce formation of paraxial mesoderm from primitive streak mesoderm. The cell culture medium in this stage is optionally supplemented with a Wnt inhibitor or a TGFβ inhibitor.

In some embodiments, the cell culture media useful for Stage III (i.e., induction of chondrocytes) are supplemented with effective amounts of combinations of small molecule(s) and protein(s) selected from: a TGFβ agonist, an FGF agonist, and a cyclic AMP agonist (e.g., Forskolin) to induce conversion of paraxial mesoderm into chondrocytes. In preferred embodiments, the cell culture media useful for Stage IV (i.e., induction of mature chondrocytes and cartilage tissues) are supplemented with a TGFβ agonist.

Methods for chemically inducing differentiation of hPSC into mature chondrocytes are also provided. Small molecule/protein combinations are used in a four-stage cell culture process, to induce formation of mature chondrocytes from hPSC. Stage I includes of hPSC in cell culture medium supplemented with effective amounts of an FGF agonist, for example, FGF; a BMP4 agonist, for example BMP4; and a TGFβ agonist, for example Activin A; for 1-6 days, preferably for about 1-3 days, to form primitive streak mesoderm. In some embodiments, the cell culture medium is also supplemented with a Wnt agonist. In some embodiments, Stage I includes the formation of hPSC in embryoid bodies. In other embodiments, Stage I does not include the formation of hPSC in embryoid bodies. Stage II includes culturing a monolayer of primitive streak mesoderm cells in cell culture media supplemented with supplemented with effective amounts of small molecule/protein combinations selected from: a BMP4 inhibitor and/or an FGF agonist (e.g., FGF), in effective amounts for about 8-14 days, preferably for about 11 days, to induce formation of paraxial mesoderm. In some embodiments, the cell culture medium is also supplemented with a Wnt antagonist or a TGFβ antagonist. Stage III includes culturing monolayer of paraxial mesoderm cells in cell culture medium supplemented with effective amounts of small molecule/protein combinations selected from: a TGFβ agonist, an FGF agonist, and a cyclic AMP agonist, for example Forskolin, for 14-40 days, preferably about 28 days, to obtain chondrocytes from a monolayer culture of paraxial mesoderm. Stage IV includes culturing chondrocytes in high density micromass or encapsulation within biomaterials to produce a cartilage tissue in cell culture media containing a TGFβ agonist.

Compositions of mature chondrocytes produced according to the disclosed methods are provided.

Methods of treating a subject in need thereof using chemically induced chondrocytes are also disclosed. The methods provide chondrocytes and/or cartilage tissues suitable for transplant. The chondrocytes and/or cartilage tissues can be used to treat or prevent one or more diseases or disorders, in a subject in need thereof. In some embodiments, the subject has osteoarthritis, osteochondritis dissecans, polychondritis, other chondropathies, or injuries or damages affecting the cartilage.

### BRIEF DESCRIPTION OF THE DRAWINGS

FIG. 1A is a schematic showing micromass cultures derived from human embryonic stem cells (HES) serially replated to make new cartilage tissues. FIG. 1B and FIG. 1C are bar graphs showing relative mRNA copy number of COL10A1 (FIG. 1B) and PRG4 (FIG. 1C) in cultures following 12 (12w) weeks in response to TGF3 and BMP in samples including unpassaged micromasses (P0), cultures serially passaged once (P1) and cultures serially passaged twice (P2) as indicated in FIG. 1A. FIG. 1D is a schematic showing the stages for differentiating pluripotent stem cells into articular chondrocytes using a micromass versus a monolayer during Stage III of the differentiation process.

FIG. 2A is a schematic showing micromass cultures derived from human embryonic stem cells (HES) serially replated and expanded (phenotype of expanded cells in micrograph) to make new cartilage tissues. FIG. 2B and FIG. 2C are bar graphs showing relative mRNA copy number of COL10A1 (FIG. 2B) and PRG4 (FIG. 2C) in cultures 12 (12w) weeks in response to TGFβ and BMP in samples including unpassaged micromasses (P0), cultures serially passaged once (E1), cultures from unpassaged micromasses that were expanded once in monolayer prior to replating (E2) or expanded twice in monolayer prior to replating (E3) as indicated in FIG. 2A.

FIGS. 3A-3C are bar graphs showing relative gene expression (mRNA copy number relative to TBP) of COL2A1 (cartilage gene) in monolayer cultures of paraxial mesoderm (day 14 of induction protocol) after 2, 3, and 4 days treated with either a combination of TGFβ, FGF and DMSO, or a combination of TGF-β. FGF and Forskolin (FSK) in which the specific components, DMSO or FSK, was added for only 1 day (1D hit), 2 days (2D hit), 3 days (3D hit) or 4 days (4D hit) (FIG. 3A); relative gene expression of COL2A1 in monolayer cultures after 2 weeks in presence of TGF-β, FGF and DMSO, or TGF-β, FGF and FSK for indicated durations (1 day (1D hit), 2 days (2D hit), 3 days (3D hit), 4 days (4D hit), or continuous (CONT)) (FIG. 3B); and gene expression of SCX in monolayer cultures after 2, 3, 4 days, or 2 weeks in presence of TGF-β, FGF and DMSO, or TGF-β, FGF and FSK FIG. 3C) for indicated durations.

FIGS. 4A-4C are bar graphs showing relative gene expression of COL2A1 (FIG. 4A), SCX (FIG. 4B), and MKX (tendon gene, FIG. 4C) of paraxial mesoderm (day 14; meso) and monolayer cultures 4 weeks in media containing FGF alone (FGF), FGF+TGFβ+Forskolin at 30 μM (FGF+TGFB+FSK30), or FGF+TGFβ+Forskolin at 100 M (FGF+TGFB+FSK100) in two starting populations of day 14 paraxial mesoderm (meso) including 420 (with BMP inhibitor and FGF from day 3-5 of differentiation, i.e., stage 2) and 420i mesoderm (420 treated with a Wnt inhibitor IWP2 from day 3-5 of differentiation, i.e., stage 2). FIG. 4D is a dot plot showing qPCR expression data for the Mohawk Homeobox gene (MKX), a tendon-associated gene, in 420 and 420i paraxial mesoderm cells plated in micromass cultures supplemented with TGFβ. qPCR was conducted 12 weeks after incubation in micromass cultures.

FIGS. 5A and 5B are bar graphs comparing the expression of COL2A1 (a cartilage associated gene, FIG. 5A) and SCX (a tendon associated gene, FIG. 5B) in 420 and 420i paraxial mesoderm cells. Cells were plated in serum-free monolayer culture with no additional factors and qPCR was performed following 4 weeks in culture. N=3 per group. FIGS. 5C and 5D are bar graphs comparing the expression of SCX (FIG. 5GC) and COL2A1 (FIG. 5D) in 420 and 420i paraxial mesoderm cells. Cells were plated in serum-free monolater culture supplemented with either FGF alone or a combination of FGF, TGFβ, and FSK. qPCR was performed following 4 weeks in culture. N=3 per group.

FIGS. 6A-6C are bar graphs showing relative gene expression of COL2A1 (cartilage gene, FIG. 6A), SCX (tendon gene, FIG. 6B), and MKX (tendon gene, FIG. 6C) of monolayer cultures after 2 weeks, 3 weeks, and 4 weeks in culture media containing no additives (SFD), DMSO alone or Forskolin alone (30 μM FSK30) without TGF-β and FGF.

FIGS. 7A-7C are bar graphs showing relative gene expression of SOX9 (cartilage gene, FIG. 7A), COL2A1 (cartilage gene, FIG. 7B), and SCX (tendon gene, FIG. 7C), of monolayer cultures in culture media, with or without bFGF, containing i) TGFβ 3, ii) TGFβ 3 and FSK (T+FSK), or iii) TGFβ 3, FSK, and a Creb binding protein inhibitor (CBPi) at a concentration of 0.1 μM, 0.5 μM, or 1.0 μM.

FIGS. 8A-8D are bar graphs showing (FIG. 8A) copy number of COL2A1 mRNA relative to TBP in tissues generated from 3-week-old cells from micromass in T15 paraxial mesoderm progenitors, Micromass (C+), RAD16-I+3w micromass [100 μl], and RAD16-I+3w micromass [200 μl]; (FIG. 81B) copy number of COL2A1 mRNA relative to TBP in tissues generated from 3-week-old cells from monolayer in paraxial mesoderm progenitors (T15), Monolayer (C+), RAD16-I+3w monolayer [100 μl], and RAD16-I+3w monolayer [200 μl]; (FIG. 8C) copy number of PRG4 mRNA relative to TBP in tissues generated from 3-week-old cells from micromass in paraxial mesoderm progenitors (T15), Micromass (C+), RAD16-I+3w micromass [100 μl], and RAD16-I+3w micromass [200 μl]; (FIG. 8D) copy number of PRG4 mRNA relative to TBP in tissues generated from 3-week-old cells from monolayer in paraxial mesoderm progenitors (T15), Monolayer (C+), RAD16-1+3w monolayer [100 μl], and RAD16-I+3w monolayer [200 μl]. The copy number of mRNA is relative to TBP as determined by qPCR-based expression analysis. T15 mesoderm indicates the starting paraxial mesoderm population which was the same for both micromass and monolayer-derived chondrocytes. Bars represent the standard error of the mean. n=number of samples per condition.

FIGS. 9A-9C are bar graphs showing relative mRNA copy number for COL10A1 (growth plate cartilage marker) (FIG. 9A), PRG4 (articular cartilage marker) (FIG. 9B) and COL2A1 (general cartilage marker) (FIG. 9C) of monolayer-derived chondrocytes (derived with either FGF-alone or the combination of TGFβ+FGF+FSK treatment) that were subsequently cultured in micromass for 6 weeks in the presence of TGFβ or BMP4 to generate articular or growth plate-like chondrocytes and cartilage tissues. FIGS. 9D-9F are bar graphs showing mRNA copy number for COL10A1 (FIG. 9D), PRG4 (FIG. 9E) and COL2A1 (FIG. 9F) of monolayer-derived chondrocytes (derived with either FGF-alone or the combination of TGFβ+FGF+FSK treatment) that were subsequently cultured in micromass (uM) for 12 weeks in the presence of TGFβ or BMP4 to generate articular or growth plate-like chondrocytes and cartilage tissues.

FIGS. 10A and 10B are principal component (based on gene expression) and gene expression plots showing that hESC-derived articular and growth plate chondrocytes have distinct transcriptional profiles similar to their respective fetal cartilage counterparts. FIG. 10A is a principal component analysis (PCA) plot of RNA-seq expression data from hESC-derived and fetal cartilages. The legend indicates cell type and sequencing batch. FIG. 10B is a graph showing the top 100 differentially-expressed genes up- and down-regulated in the hESC-derived cartilages (top) compared with equivalent log(2)FC values from the fetal cartilage (bottom).

FIGS. 11A-11P are dot plots illustrating results from the validation of differential gene expression in hESC-derived articular and growth plate cartilage and fetal epiphyseal and growth plate cartilage. FIGS. 11A-11H show quantitative RT-PCR data of the differentially expressed genes (DEGs) in hESC-derived cartilage: FGF18 (FIG. 11A), PTHLH (FIG. 11B), MEOX1 (FIG. 11C), CHI3L1 (FIG. 11D), PTH1R (FIG. 11E), FGFR3 (FIG. 11F), PANX3 (FIG. 11G), and ALPL (FIG. 11H). N=5 independent experiments with 3-6 replicates per experiment. FIGS. 11I-11P show quantitative RT-PCR data of the differentially expressed genes (DEGs) in fetal cartilage: FGF18 (FIG. 11I), PTHLH (FIG. 11J), MEOX1(FIG. 11K), CHI3L1 (FIG. 11L), PTH1R (FIG. 11M), FGFR3 (FIG. 11N), PANX3 (FIG. 11O), and ALPL (FIG. 11P). Chondrocytes were isolated from the epiphysis and growth plate of the distal femur and proximal tibia (N=3 per site at E59, E67 and E72). *p<0.05, **p<0.01, ***p<0.001.

FIG. 12A is a histogram showing the expression and overlap in expression of a subset of transcription factors (TFs) between hESC-derived and human fetal chondrocytes. FIG. 12A shows the direction of the top 20 differentially-expressed transcription factors up- and down-regulated when comparing HESC-derived articular and growth-plate chondrocytes (top), along with the equivalent log(2)FC values from the fetal tissue samples (bottom). FIGS. 12B and 12C are representative tables showing transcription factor motif enrichments within the epigenetic profiles of hESC-derived articular chondrocytes (ACs) and growth plate cells (GPCs). HOMER de-novo motif enrichments for putative enhancer sequences biased towards either TGFβ or BMP-treated lineages. Best-matched motif to de-novo results are indicated; BMP4 homer motifs (FIG. 12B) and TGFβ homer motifs (FIG. 12C).

FIG. 13A is a graph illustrating results suggesting that the variance in expression of genes can be attributed to different classes of regulatory elements (gene regulatory behavior). In FIG. 13A, logFC values of genes clustered by regulatory behavior. Significance bars indicate Tukey post-hoc corrected p-values. Proportion of significant differentially expressed (DE) genes in each cluster are indicated. n.s., not significant; *p<0.05; **p<0.01; ***p<0.001. FIGS. 13B-13G are graphs identifying putative lineage-delineating transcription factors. Enrichment test results comparing the occurrence of the indicated motif in TGFβ or BMP-biased DARs relative to randomized backgrounds. Top five motifs (ordered by difference in enrichment results) shown for each category. FIG. 13B is a bar graph showing TFs differentially expressed (DE) in TGFβ-treated articular chondrocytes, testing motif occurrence in TGFβ or BMP-biased DARs around enhancer-centric DEGs. FIG. 13C is a bar graph showing TFs DE in TGFβ-treated articular chondrocytes, testing motif occurrence in TGFβ or BMP-biased DARs around combo-centric DEGs. FIG. 13D is an enrichment histogram of RELA motif occurrence in BMP (left) and TGFβ (right)-biased DARs around combo-centric genes DE in their respective lineages. Red line indicates target set value, black bars indicate occurrences in randomized sets. FIG. 13E is a bar graph showing the TFs DE in BMP-treated growth plate chondrocytes, testing motif occurrence in TGFβ or BMP-biased DARs around enhancer-centric DEGs. FIG. 13F is a bar graph showing TFs DE in BMP-treated growth plate chondrocytes, testing motif occurrence in TGFβ or BMP-biased DARs around combo-centric DEGs. FIG. 13G is an enrichment histogram of RUNX2 motif occurrence in BMP (left) and TGFβ (right)-biased DARs around combo-centric genes DE in their respective lineages. Red line indicates target set value, black bars indicate occurrences in randomized sets. *p<0.05; NS, not significant.

FIGS. 14A-14S are scatter dot plots showing the putative targets of TF regulation in hESC-derived articular and growth plate chondrocytes. FIG. 14A illustrates results that RELA is differentially expressed in TGFβ-treatment. FIGS. 14B-14H illustrates the expression of selected genes with putative RELA binding motifs quantified by qRT-PCR. The selected genes are GLIPR2 (FIG. 14B), LOXL2 (FIG. 14C), PRG4 (FIG. 14D), DKK3 (FIG. 14E), TLR2 (FIG. 14F), LTBP2 (FIG. 14G), and COL15A1 (FIG. 14H). *p<0.05, **p<0.01, ***p<0.001. FIG. 14I illustrates results showing that RUNX2 is differentially expressed in BMP-treatment. FIGS. 14J-14S show the expression of selected genes with putative RUNX2 binding motifs was quantified by qRT-PCR. The selected genes are ATOH8 (FIG. 14J), ACAN (FIG. 14K), C16ORF72 (FIG. 14L), COL10A1 (FIG. 14M), RCL1 (FIG. 14N), WNT10B (FIG. 14O), and GRP153 (FIG. 14P), MAP4K3 (FIG. 14Q), and RXRA (FIG. 14R), and SCUBE (FIG. 14S. *p<0.05, **p<0.01, ***p<0.001.

FIGS. 15A-15C are graphs showing the TF interaction with putative regulatory elements was validated by ChIP-qPCR. FIGS. 15A and 15B illustrate ChIP-qPCR of RELA results for differentially accessible peaks near target genes show enrichment of these sequences compared to a negative (gene desert) control (Untr12). BIRC3 is a positive RELA control. FIGS. 15A and 15B represent two pools of TGFβ-treated articular chondrocytes for ChIP. FIG. 15C shows ChIP-qPCR of RUNX2 results for differentially accessible peaks near target genes show enrichment of these sequences compared to a negative (gene desert) control (Untr12). DPF1 is a positive RUNX2 control.

FIG. 16A shows copy number of COL2A1 mRNA relative to TBP and FIG. 16B shows copy number of PRG4 mRNA relative to TBP in 6-week-old tissues generated from micromass cells or monolayer-derived cells either by replating into micromass culture (replated uM) or by encapsulation in RAD16-I [100 μl]. The copy number of mRNA is relative to TBP as determined by qPCR-based expression analysis. T15 mesoderm indicates the starting paraxial mesoderm population which was the same for both micromass and monolayer-derived chondrocytes. Bars represent the standard error of the mean. n=number of samples per condition. FIG. 16C-16F depict copy number mRNA in stage III paraxial mesoderm (day 14), micromass cultures derived from paraxial mesoderm plated into high densitty micromass at stage III (Micromass), monolayer cultures derived from paraxial mesoderm plated in monolayer at stage III (Monolayer), and Monolayer-derived micromass cultures derived from monolayer cells plated into micromass culture after 4 weeks, after 1 week, 2 weeks, 3 weeks, 4 weeks, as indicated.

FIGS. 17A-17C depict copy number COL10A1 mRNA normalized to TBP in micromass cultures at indicated timepoints and treatment regimens. FIG. 17A depicts copy number COL10A1 mRNA in micromass cultures treated with TGFB3 from 2-24 weeks, and in TGFB3-treated micromass cultures that were switched to BMP4-supplemented media after 2 weeks (BMP4 @2w), 2 weeks, and 4, 6, 8, 10, 12 weeks in which gene expression was quantified after 2, 6, 12, or 24 weeks after switching media to BMP4-supplementation. FIG. 17B and FIG. 17C depict copy number PRG4 (FIG. 17B) and COL10A1 (FIG. 17C) in micromass cultures after 2, 6, 12, and 24 weeks (w).

FIG. 18A shows examples of quantified amount of sulfated (s) glycosaminoglycans (GAG) (μg per μg of DNA content) in micromass cultures cultured in the presence of TGFβ3 or BMP4 for indicated times (weeks). sGAG content increases over time in both cartilaginous tissues. FIG. 18B depicts representative quantification of both sulfated GAG and hydroxy-proline (OH-Pro; a surrogate biochemical quantification of collagen content), in TGFβ3-treated articular cartilage tissues cultured for 12 weeks. Values were calculated as μg per μg of DNA content per culture. Error bars represent standard error of the mean. FIG. 18C depicts the relative differential expression levels of representative collagen genes in articular (TGFβ) and growth plate (BMP) cartilage micromass tissues. Values represent mean (n=6) counts from RNA-sequencing, error bars indicate standard deviation. Graph depicts only those genes that were found to be significantly differentially expressed, i.e., non-inclusive of all collagen genes expressed in these tissues.

### DETAILED DESCRIPTION OF THE INVENTION

### I. Definitions

The term “primitive streak-like mesoderm cell population” as used herein means a population of mesoderm cells expressing Brachyury and the cell surface markers CD56 and PDGFRα. For example, the primitive streak-like mesoderm cell population can comprise at least 50%, at least 60%, at least 70%, at least 80% or about 90% cells expressing CD56 and PDGFRα Cartilage differentiation has been obtained with the disclosed methods using for example 50% CD56/PDGFRα+ cells.

The term “paraxial mesoderm cells” refer to a population of mesoderm cells expressing cell surface CD73, CD105 and/or PDGFR-beta. For example, the paraxial mesoderm cell population comprises at least 70% cells expressing, CD73, CD105 and/or PDGFR-beta.

The term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCR, in situ hybridization, Western blotting, and immunostaining such as FACS.

The term “expressing” also represented as “+” means, with respect to a cell protein level, detectable protein expression compared to a cell that is not expressing the protein, for example as measured by FACS analysis.

The term “culturing” as used herein incubating and/or passaging cells in an adherent, suspension or 3D culture. As used herein, the term “adherent culture” refers to a cell culture system whereby cells are cultured on a solid surface, which may in turn be coated with an insoluble substrate that may in turn be coated with another surface coat of a substrate, such as those listed below, or any other chemical or biological material that allows the cells to proliferate or be stabilized in culture. The cells may or may not tightly adhere to the solid surface or to the substrate.

The term “contacting” or “culturing . . . with” is intended to include incubating the component(s) and the cell/tissue together in vitro (e.g., adding the compound to cells in culture) and the step of “contacting” or “culturing . . . with” can be conducted in any suitable manner. For example, the cells may be treated in adherent culture, in suspension culture, or in 3D culture; the components can be added temporally substantially simultaneously (e.g., together in a cocktail) or sequentially (e.g., within 1 hour, 1 day or more from an addition of a first component). The cells can also be contacted with another agent such as a growth factor or other differentiation agent or environments to stabilize the cells, or to differentiate the cells further and include culturing the cells under conditions known in the art.

The term “serum-free” refers to the absence of serum in the solutions e.g., medias used to culture the given cell population. For example, serum free medium or environment can contain less than 4, 3, 2, or 1% serum. In a preferred embodiment, the serum free composition does not contain serum, or only contains trace amounts of serum from the isolation of components that are added to the defined media (e.g., contains 0% added serum).

The term “BMP inhibitor” means any inhibitor of BMP signaling and includes, for example, a type 1 BMP receptor inhibitor, BMP ligands and/or soluble BMP receptors, such as dorsomorphin (DM), noggin, Chordin, LDN-193189, soluble BMPR1a, and/or soluble BMPR1b.

The term “nodal agonist” as used herein means any molecule that activates nodal signal transduction such as “nodal” (for example human nodal such as Gene ID: 4338) or “activin” in a hepatocyte lineage cell.

The term “agonist” means an activator, for example, of a pathway or signaling molecule. An agonist of a molecule can retain substantially the same, or a subset, of the biological activities of the molecule (e.g., nodal). For example, a nodal agonist means a molecule that selectively activates nodal signaling.

The term “inhibitor” means a selective inhibitor, for example, of a pathway or signaling molecule. An inhibitor or antagonist of a molecule (e.g., BMP4 inhibitor) can inhibit one or more of the activities of the naturally occurring form of the molecule. For example, a BMP4 inhibitor is a molecule that selectively inhibits BMP4 signaling.

The term “selective inhibitor” means the inhibitor inhibits the selective entity or pathway at least 1.5×, 2×, 3×, 4× or 10× more efficiently than a related molecule.

The term “specifying” means a process of committing a cell toward a specific cell fate, prior to which the cell type is not yet determined and any bias the cell has toward a certain fate can be reversed or transformed to another fate. Specification induces a state where the cell's fate cannot be changed under typical conditions. Specification is a first step of differentiation but can also refer to the differentiation of cells derived in the first step in subsequent steps or stages.

The term “stem cell” refers to an undifferentiated cell which is capable of proliferation, self-renewal and giving rise to more progenitor or precursor cells having the ability to generate a large number of mother cells that can in turn give rise to differentiated, or differentiable, daughter cells. The daughter cells can for example be induced to proliferate and produce progeny cells that subsequently differentiate into one or more mature cell types, while also retaining one or more cells with parental developmental potential. The term “stem cell” includes embryonic stem cell and pluripotent stem cell.

The term “embryonic stem cell” refers to the pluripotent stem cells of the inner cell mass of the embryonic blastocyst (see, for example, U.S. Pat. Nos. 5,843,780, 6,200,806). Such cells can also be obtained from the inner cell mass of blastocysts derived from somatic cell nuclear transfer (see, for example, U.S. Pat. Nos. 5,945,577, 5,994,619, 6,235,970).

The term “pluripotent stem cell” refers to a cell with the capacity, under different conditions, to differentiate to more than one differentiated cell type, and, for example, the capacity to differentiate to cell types having characteristic of the three germ cell layers. Pluripotent cells are characterized by their ability to differentiate to more than one cell type using, for example, a nude mouse teratoma formation assay. Pluripotency is also evidenced by the expression of embryonic stem (ES) cell markers. Pluripotent stem cells include induced pluripotent stem cells (iPSC) and embryonic stem cells. In an embodiment, the pluripotent stem cell is derived from a somatic cell. In an embodiment, the pluripotent stem cell is derived from a human somatic cell.

The terms “iPSC” and “induced pluripotent stem cell” are used interchangeably and refers to a pluripotent stem cell artificially derived (e.g., induced or by complete reversal) from a non-pluripotent cell, typically an adult somatic cell, for example, by inducing expression of one or more genes including POU4F1/OCT4 (Gene ID; 5460) in combination with, but not restricted to, SOX2 (Gene ID; 6657), KLF4 (Gene ID; 9314), cMYC (Gene ID; 4609), NANOG (Gene ID; 79923), LIN28/LIN28A (Gene ID; 79727)). The expression can be induced for example by forced gene expression or using small molecules, small RNAs, non-integrating gene expression vectors, or proteins.

The term “chondrocyte like cells” means chondrocyte cells and cells that are cytochemically similar and express chondrocyte markers, including for example Sox9 and Collagen 2, and behave as chondrocyte cells. The chondrocyte cells can be articular cartilage like chondrocytes or precursors or chondrocytes that are capable of hypertrophy (optionally referred to as Growth plate chondrocyte (GPC)-like cells) or precursors thereof.

The term “cartilage-like tissue” means cartilage tissue and tissue that is histologically similar and expresses cartilage markers, for example, collagen 2 and aggrecan, and behaves as cartilage, including articular cartilage tissue and/or growth plate cartilage-like tissue.

The term “articular chondrocyte like cells and/or cartilage tissue” means a population, optionally enriched or mixed, comprising articular chondrocyte cells and/or articular chondrocyte-like cells including for example, cartilage like tissue comprising articular chondrocyte-like cells.

The term “hypertrophic chondrocyte like cells and/or cartilage tissue” or “GPC like cells and/or cartilage tissue” means a population, optionally enriched or mixed, comprising hypertrophic chondrocyte cells and/or hypertrophic chondrocyte like cells (e.g., chondrocytes within the growth plates of developing bones) including, for example, cartilage like tissue comprising hypertrophic chondrocyte like cells.

The term “articular cartilage like tissue” or “cartilage containing non-hypertrophic chondrocyte-like cells” is histologically similar and expresses articular cartilage markers such as lubricin (PRG4) and/or CILP2 and behaves as articular cartilage. For example, articular cartilage is maintained as stable cartilage in vivo.

The term “growth plate cartilage like tissue” as used herein means cartilage tissue that is histologically similar and expresses cartilage markers that are found in growth plate cartilage tissue including COL10A1, RUNX2, SP7 and/or ALPL and behaves like growth plate cartilage. For example, growth plate cartilage functions in vivo to provide a scaffold onto which new bone will form.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 65%, preferably at least about 75%, at least about 85%, more preferably at least about 90%, and most preferably at least about 95% pure, with respect to the cells making up a total cell population.

The terms “enriching” or “enriched” are used interchangeably herein and mean that the yield (fraction) of cells of one type is increased by at least about 10%, at least about 20%, at least about 30%, at least about 40%, at least about 50% or at least about 60% over the fraction of cells of that type in the starting culture or preparation. Enriching and partially purifying can be used interchangeably.

The population of cells can be enriched using different methods such as methods based on markers such as cell surface markers (e.g., FACS sorting etc.).

The term “subject” as used herein includes all members of the animal kingdom including mammals such as and including a primate such as human, monkey or ape, domestic pets, livestock, and laboratory animals.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to a subject.

The term “treatment” as applied to a subject, refers to an approach aimed at obtaining beneficial or desired results, including clinical results and includes medical procedures and applications including for example pharmaceutical interventions, surgery, radiotherapy, and naturopathic interventions as well as test treatments for treating joint/bone disorders. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable.

The terms “administering”, “implanting” and “transplanting” are used interchangeably in the context of delivering cells tissues and/or products described herein into a subject, by a method or route which results in at least partial localization of the introduced cells at a desired site. The cells can be implanted directly to a joint, or alternatively be administered by any appropriate route which results in delivery to a desired location in the subject where at least a portion of the implanted cells or components of the cells remain viable.

“Pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual.

The term “reduce”, “inhibit”, “alleviate” or “decrease” are used relative to a control, either no other treatment or treatment with a known degree of efficacy. One of skill in the art would readily identify the appropriate control to use for each experiment. For example, a decreased response in a subject or cell treated with a compound is compared to a response in subject or cell that is not treated with the compound.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., injury size/type, age, joint health, immune system health, etc.), the disease or disorder, and the treatment being administered. The effective amount can be relative to a control. Such controls are known in the art and discussed herein, and can be, for example the condition of the subject prior to or in the absence of administration of the drug, or drug combination, or in the case of drug combinations, the effect of the combination can be compared to the effect of administration of only one of the drugs.

“Excipient” is used herein to include a compound that is not a therapeutically or biologically active compound. As such, an excipient should be pharmaceutically or biologically acceptable or relevant, for example, an excipient should generally be non-toxic to the subject. “Excipient” includes a single such compound and is also intended to include a plurality of compounds.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### II. Compositions

Human pluripotent stem cell (hPSC)-derived articular chondrocytes with desirable features for use in cartilage tissue engineering are disclosed. Formulations containing chondrocytes produced according to methods, and one or more excipients are also provided herein. Agents for use in chemical differentiation of hPSC cells into functional chondrocytes are also provided.

Cell culture media compositions supplemented with small molecules/protein which can be used to facilitate the chemical differentiation of human pluripotent stem cells (hPSCs) into articular chondrocytes are provided.

### A. Cell Culture Media

Cell culture media compositions containing combinations of chemical compounds which can be used to induce the chemical differentiation of human pluripotent stem cells (hPSCs) partially or completely into articular chondrocytes are disclosed. The required combinations of small molecules/protein can vary depending on stage of chondrogenesis.

The cells culture media includes base media supplemented with small molecule factors/proteins as disclosed herein. In an embodiment, optionally during stages 3 and/or 4 the media is serum free and comprises a base media optionally, high glucose DMEM supplemented with dexamethasone, ascorbic acid, insulin, transferrin, selenium, and proline.

As used herein, a base media refers to a mixture of salts that provide cells with water and certain bulk inorganic ions essential for normal cell metabolism, maintain intra- and extra-cellular osmotic balance. provide a carbohydrate as an energy source. and provide a buffering system to maintain the medium within the physiological pH range. Examples of base medias include, but are not limited to, Dulbecco's Modified Eagle's Medium (DMEM), Minimal Essential Medium (MEM), Basal Medium Eagle (BME), RPMI 1640, Ham's F-10, Ham's F-12, alpha-Minimal Essential Medium (aMEM), Glasgow's Minimal Essential Medium (0-MEM), and Iscove's Modified Dulbecco's Medium (IMDM), STEM PROR, STEM PRO-34® and mixtures thereof.

### 1. Cell Culture Media for Stage I

First for induction of hPSC cells to form Primitive streak rnesoderm, the required combination of active signals includes: (A) an FGF agonist. (B) a BMP4 agonist. and (C) a TGFβ agonist, and optionally, Wnt agonist, at effective levels to induce induction of hPSC cells to form Primitive streak mesoderm. These signals can be endogenously made by hPSC-derived cells, or they can be induced through the supplementation of molecules including (A) an FGF agonist, (B) a BMP4 agonist, and (C) a TGF3 agonist, and optionally, Wnt agonist, at effect amounts to induce induction of hPSC cells to form Primitive streak mesoderm.

### (a) FGF Agonist

In preferred embodiments, the FGF agonist is a fibroblast growth factor agonist. The FGF agonist may be a molecule such as a cytokine, for example FGF. “FGF” as used herein, refers to any fibroblast growth factor, and optionally bFGF, FGF2, FGF4, FGF9 and/or optionally FGF 19, 21, 3, 5, 6, 8a, 16-18, 20 and/or 23, for example human FGF1 (Gene ID: 2246), FGF2 (also known as bFGF; Gene ID: 2247), FGF3 (Gene ID: 2248), FGF4 (Gene ID: 2249), FGF5 (Gene ID: 2250), FGF6 (Gene ID: 2251), FGF7 (Gene ID: 2252). FGF8 (Gene ID: 2253), FGF9 (Gene ID: 2254) and FGF10 (Gene ID: 2255) optionally including active conjugates and fragments thereof, including naturally occurring active conjugates and fragments. In some embodiments, FGF is βFGF, FGF2, FGF4, and/or FGF9. As used herein, “active conjugates and fragments of FGF” include conjugates and fragments of a fibroblast growth factor that bind and activate a FGF receptor and optionally activate FGF signaling. In some forms, the FGF agonist is a molecule that activates a FGF signaling pathway, i.e., binds and activates a FGF receptor. Preferably the FGF agonist is FGF2 or FGF. Preferably, the FGF agonist FGF is any concentration between about 0.1 ng/ml and about 20 ng/ml, optionally about 5 ng/ml.

### (b) BMP4 Agonist

In some embodiments, the 3MP4 agonist is a bone morphogenic protein 4 agonist and includes any BMP or GDF that activates the receptor for BMP4. The term “BMP4” (for example Gene ID: 652) as used herein, refers to Bone Morphogenetic Protein 4, for example human BMP4, as well as active conjugates and fragments thereof, optionally including naturally occurring active conjugates and fragments, that can for example activate BMP4 receptor signaling. BMP4 agonists include but are not limited to GDF5, GDF6, GDF7, BMP4, BMP2, BMP6, BMP7 and/or BMP10. Preferably, the BMP4 agonist is BMP4 is any concentration between about 0.1 ng/mil and about 100 ng/m, optionally about 3 ng/ml.

### (c) TGFβ Agonist

TGFβ, agonist refer to any molecule that activates the TGFβ receptor TGFβ receptors are single pass serine/threonine kinase receptors. Three TGFβ receptor types include receptor ty pes I, II and III i.e., TGF receptor 1, TGF P receptor 2, and TGFβ receptor 3. Preferably, the TGFβ agonist is TGFβ 3 or Activin A. Examples of Activin A include but are not limited to GENE ID: 3624 (human activin), as well as active conjugates and fragments thereof, optionally including naturally occurring active conjugates and fragments, that can for example activate nodal signal transduction as well as active conjugates and fragments thereof, including naturally occurring active conjugates and fragments. Preferably, the TGFβ agonist is Activin A in any concentration between about 0.1 ng/ml and about 100 ng/ml, optionally about 2 ng/ml.

### (d) Wnt Agonist

Useful Wnt (Wingless and Int-1) agonist are molecules that activates Wnt/beta-catenin receptor signaling in a chondrocyte lineage cell and includes, for example, Wnt3a and as well as GSK3 selective inhibitors such as CHTR99021 (STEMOLECULE™ CHIR99021 Stemgent), 6-Bromolndirubin-3′-Oxime (BIO) (Cayman Chemical (cat:13123)), or STEMOLECULE™ BIO from Stemgent (cat:04003). CHIR99021 is a selective inhibitor of GSK3. The GSK3 selective inhibitors contemplated are for example selective inhibitors for GSK-3α/β in the Wnt signaling pathway. Wnt3a as used herein refers to wingless-type MMTV integration site family, member 3A factor (e.g., Gene ID: 89780), for example human Wnt3a, as well as active conjugates and fragments thereof, including naturally occurring active conjugates and fragments. Other Wnt agonists include but are not limited to SB216763, TWS119, CHIR98014, Tideglusib, SB415286, LY2090314, CHIR-98014, AZD1080, TDZD-8 and wnt3a.

### 2. Cell Culture Media for Stage II

Second, to induce formation of the paraxial mesoderm from Primitive streak mesoderm, the required combination of molecules includes effective amounts of (A) a BMP4 inhibitor and/or (B) a FGF agonist. Optionally, a Wnt inhibitor and a TGFβ inhibitor is used in effective amounts.

### (a) BMP4 Inhibitor

A preferred BMP4 inhibitor is dorsomorphin used in a concentration ranging from about 0.5 μM and optionally about 6 μM, preferably 4 μM. Other molecules which an inhibit BMP4 signaling are known in the art and include, but at not limited to LDN 193189 dihydrochloride.

### (b) FGF Agonist

Useful FGF agonists the same as disclosed above for the Stage I medium. A preferred FGF agonist useful for supplementing the stage II medium is basic FGF/FGF2 used in a concentration ranging from about 1 ng/mL to about 100 ng/mL, from about 10 to about 50 ng/ml, preferably about 20, 30, or 40 ng/ml, with the intervening numbers contemplated.

### (c) Wnt Inhibitor/Antagonist

Useful Wnt antagonists or Wnt inhibitors refers to molecules that inhibits Wnt/beta catenin receptor signaling in a chondrocyte lineage cell, including for example IWP2 (N-(6-Methyl-2-benzothiazolyl)-2-[(3,4,6,7-tetrahydro-4-oxo-3-phenylthieno[3,2-d]pyrimidin-2-yl)thio]-acetamide; Sigma); Dickkopf-related protein 1 (DKK1; R & D Systems), Wnt-C59 (4-(2-Methyl-4-pyridinyl)-N-[4-(3-pyridinyl)phenyl]benzeneacetamide) and/or XAV939 (3,5,7,8-Tetrahydro-2-[4-(trifluoromethyl)phenyl]-4H-thiopyrano[4,3-d]pyrimidin-4-one; Sigma). A preferred Wnt inhibitor is IWP2 used in a concentration ranging from 0.5 μM and about 4 μM, preferably 2 μM.

### (d) TGFβ Inhibitor

A preferred TGFβ inhibitor is the inhibitor of type I activin receptor-like kinase (ALK) receptors SB431542. SB431542 is used in a concentration ranging from about 0.5 μM and about optionally about 10 μM, preferably 5.4 M. Other exemplary molecules which can inhibit ALK receptors include GW788388 and A-83-01.

### 3. Cell Culture Media for Stage III

Third, to convert paraxial mesoderm cells into chondrogenic progenitors, a cocktail containing one or a combination of (1) an FGF agonist, (2) a TGFβ agonist, and (3) a cyclic AMP agonist in effective amounts.

### (a) TGFβ Agonist

Useful TGFβ agonist the same as disclosed above for the Stage I medium. A preferred TGFβ agonist useful for supplementing the stage III medium is TGFβ3 used in a concentration ranging from about 1 ng/ml and about 50 ng/ml, optionally about 10 ng/ml.

### (b) FGF Agonist

Useful FGF agonists the same as disclosed above for the Stage I medium. A preferred FGF agonist useful for supplementing the stage III medium is FGF used in a concentration ranging from 1 ng/ml to 100 ng/ml, preferably 10 ng/ml.

### (c) Cyclic AMP Agonist

In preferred embodiments, molecules that improve or boost differentiation of chondrocytes are included. Preferably, the molecules that improve or boost differentiation are cyclic AMP (cAMP) agonists. Non-limiting examples of cyclic AMP agonists include prostaglandin E2 (PGE2), dibutyryl cyclic-AMP (dbcAMP), 8-Br-cAMP, genistein, Forskolin (FSK), colforsin, and roliprarn. Preferably, the cyclic AMP agonist used to improve differentiation of chondrocyte precursors into chondrocytes is forskolin. A preferred cAMP agonist is Forskolin used in a concentration ranging from 5 μM and about 100 M, optionally about 30 μM.

### 4. Cell Culture Media for Stage IV

In some embodiments, to generate articular cartilage tissues from chondrocytes, a cocktail of media containing a TGFβ agonist is used.

### (a) TGFβ Agonist

Useful TGFβ agonist are the same as disclosed above for the Stage I medium. A preferred TGFβ agonist useful for supplementing the stage III medium is TGFβ3 used in a concentration ranging from about 1 ng/ml and about 50 ng/ml, preferably about 10 ng/ml.

### B. Chemically Induced Chondrocytes and Formulation

### 1. Chemically Induced Chondrocytes

The articular chondrocytes are preferably derived from human pluripotent stem cells (hPSCs). However, the articular chondrocytes can be derived from an animal PSCs, including, but not limited to, dog, horse, pig primates such as monkey and chimpanzee. The PSCs can be embryonic stem cells (ESCs) or induced pluripotent stem cells (iPSCs). The ESCs can be “true” ESCs derived from the inner cell mass of embryos, ESCs made by somatic cell nuclear transfer.

In some forms, the derived articular chondrocytes express one or more markers associated with a general cartilaginous phenotype, such as ACAN, SOX9, COL2A1, ACAN, COL9A1, SOX5, and SOX6. Additional markers specific for articular chondrocytes include PRG4, COL1A1, CILP2, COL22A1, COL15A1, FGF18, COMP, PTHLH, FGF1, ERG, COL6A1, UCMA, LECT1/CNMD, CHI3LT, CHI3L2.

The population of articular chondrocytes can be isolated from the stem cell- or progenitor cell-derived paraxial mesoderm cell culture by selecting cells that express one or more markers associated with a cartilaginous phenotype, e.g., ACAN, SOX9, COL2A1, COL9A1, SOX5, PRG4 and SOX6. This process ensures that no other types of cells are present in the population of articular chondrocytes.

In some embodiments, the population of hPSC-derived articular chondrocytes cells are in arrested superficial zone or intermediate zone-like states with less than 10% that continue differentiating/proliferating. In some embodiments, more than about 20% of the cells are in an arrested superficial zone or like state, (5-40%). In some embodiments, more than about 60% of the cells are in an arrested intermediate zone-like state (30-95%).

In some embodiments, the pluripotent cells and/or their progeny are autologous. In some embodiments, the pluripotent cells and/or their progeny are allogeneic. In some embodiments, the pluripotent cells and/or their progeny are syngeneic. In some embodiments, the pluripotent cells and/or their progeny are xenogeneic.

a. Structure of hPSC-Derived Articular Chondrocytes

Chondrocytes are specialized mesenchymal cells that occupy approximately 1-10% of the total tissue volume of the articular cartilage. Chondrocytes have different morphologies due to their different locations in cartilage. Superficial zone chondrocytes are located in the surface layer of cartilage tissue. They are distributed individually with a small volume and an oval shape. The long axis is parallel to the surface of the cartilage. Developmentally, cells that are present at the superficial zone of the cartilage can give rise to chondrocytes in deeper zones. Chondrocytes in the intermediate zone of articular cartilage, just below the superficial zone, are generally evenly distributed within the extracellular matrix. Under the electron microscope, chondrocytes have a large number of rough endoplasmic reticulum, a developed Golgi complex, and a small amount of mitochondria in the cytoplasm. Chondrocytes are buried in the cartilage interstitium. It is located in a small cavity called a cartilage lacuna.

Adult articular cartilage has a highly organized structure with 4 zones: the superficial (tangential zone); transitional (middle/intermediate) zone; deep (basal) zone, and calcified zone. Chondrocyte phenotype, density and cell shape vary among the 4 zones. In some embodiments, the derived articular chondrocytes in the superficial zone account for 5-25% of articular cartilage volume. In some embodiments, the derived articular chondrocytes in the transitional/intermediate zone account for 75-95% of articular cartilage volume. In some embodiments, the derived articular chondrocytes in the deep zone account for <5% of articular cartilage volume. There exist no calcified cartilage cells in the derived articular cartilage tissues.

Typically, naturally existing chondrocytes in the superficial zone of articular cartilage are elongated and flattened. The transitional zone typically contains more roundly shaped chondrocytes, and the deep zone contains spherical chondrocytes. In some embodiments, 5-40% of the derived articular chondrocytes are elongated and flattened. In some embodiments, 30-95% of the derived articular chondrocytes are roundly shaped. In some embodiments, <5% of the derived articular chondrocytes are spherical.

b. Function of hPSC-Derived Articular Chondrocytes

Articular chondrocytes produce and maintain the cartilaginous matrix, which consists mainly of collagen and proteoglycans. In some embodiments, the articular chondrocytes are metabolically active cells that synthesize and turnover extracellular matrix (ECM) components such as collagen, glycoproteins, proteoglycans, and hyaluronan. In some forms, 70-100% of the derived articular chondrocytes are expressing ECM components such as collagens and proteoglycans

In some forms, the derived-articular chondrocytes maintain cartilage homeostasis by producing enzymes, growth factors and inflammatory mediators. The functions of derived articular chondrocytes are similar to those chondrocytes in developing human articular cartilage. These functions are considerably higher than chondrocytes derived from PSCs using other methods, as they produce ECM and a cartilage tissue that is similar to native cartilage. In preferred embodiments, hPSC-derived articular chondrocytes tissues have abundant sulfated glycosaminoglycans (sGAGs), more preferably at a level comparable to range within human articular cartilage.

Articular chondrocytes remodel and degrade ECM constituents by synthesizing and secreting proteinases necessary for tissue remodeling. Among the proteinases responsible for cleavage of collagen and proteoglycans are matrix metalloproteinases (MMPs) and disintegrin-metalloproteinases with thrombospondin motifs (ADAMTS), respectively, and to a lesser extent, other types of enzymes such as elastase and cathepsin.

i. Formulations

In some embodiments, the formulation includes articular chondrocytes provided in a cell culture media. In some forms, the number of cells in the formulation is between about 1 and 100 million cells, between about 5 and 50 million cells, or preferably about 5 million cells.

In some embodiments, the formulation includes articular chondrocytes provided in a cryopreservative. In some forms, the number of cells in the formulation is between about 1 and 30 million cells, preferably 10 million cells.

Media for preservation of cells are known in the art, for example, CRYO-GOLD™ (cryopreservation medium) and CROSSTOR® (cyropreservation freeze media) designed to mitigate temperature-induced molecular cell stress responses during freezing and thawing. All CROSSTOR® products are pre-formulated with USP grade DMSO, a permeant solute cryoprotective agent which helps mitigate damage from the formation of intracellular ice. CROSSTOR® is offered in several packages and pre-formulated with DMSO in final concentrations of 2%, 5%, and 10%. A preferred medium for cell preservation includes 5-10% DMSO, for example, CRYOSTOR® CS10 (a uniquely formulated serum-free, animal component-free, and defined cryopreservation medium containing 10% dimethyl sulfoxide (DMSO)). Additionally, cryoprotectants/cryoprotectant additives which can be include in a cell composition (for cryopreservation) are known in the art and include, ethylene glycol (EG), antioxidants such as taurine, Metformin, gamma amino butyric acid (GABA). Cells may be suspended in a “freeze medium” such as cell culture medium containing 15-20% fetal bovine serum (FBS) and 7-10% DMSO, with or without 5-10% glycerol, at a density, for example, of about 1-10×10⁶ cells/ml. The cells are dispensed into glass or plastic vials, which are then sealed and transferred to a freezing chamber of a programmable or passive freezer. The optimal rate of freezing may be determined empirically. For example, a freezing program that gives a change in temperature of −1° C./min through the heat of fusion may be used. Once vials containing the cells have reached −80° C., they are transferred to a liquid nitrogen storage area.

In one preferred embodiment, the cryopreservation media includes about 50% FBS, about 10% DMSO and about 40% IMDM (Iscove's Modified Dulbecco's Medium).

c. Articular Chondrocytes and ECM

In some embodiments, the method is used to produce a cartilage repair implant comprising an extracellular matrix (ECM) and a population of articular chondrocytes as disclosed herein. In some embodiments, articular chondrocytes are suspended in a media or biomaterial composition injectable solution whose formulation is between 5 and 100 million cells per milliliter, preferably 12.5 million cells. The injectable material could contain biomaterials such as collagens, polyglycolic acid (pga), polylactic acid, alginates (for example, the calcium salt), polyethylene oxide, fibrin adhesive, polylactic acid-polyglycolic acid copolymer, proteoglycans, glycosaminoglycans, natural biomaterials such as matrigel, chondrocyte-derived extracellular matrix that has been partially or fully disrupted by enzymes, or synthetic components such as RAD-161 (puramatrix).

In some embodiments, the formulation contains a population of articular chondrocyte cells surrounded by an extracellular matrix (ECM). In some forms, the articular chondrocytes constitute 2-10% of cartilage tissue volume. In some forms, the number of cells in the formulation is between about 0.4 and 100 million cells, preferably about 5 million cells. In some forms, the ECM constitutes 90% of the cartilage tissue volume.

In some embodiments, the formulation of articular chondrocyte cells with ECM is an organized structure with 2 zones: the superficial (tangential zone) and the transitional (middle/intermediate) zone. In some forms, the articular chondrocyte phenotype, density, and cell shape vary among the 2 zones. In some embodiments, the superficial zone accounts for 5-25% of articular cartilage volume. In some embodiments, the transitional zone is the thickest layer, accounting for 75-95% of articular cartilage volume. In some embodiments, the cartilage contains additional zones of cartilage: deep zone chondrocytes and calcified chondrocytes. The derived deep zone may account for 10-30% of articular cartilage volume.

In some embodiments, the superficial zone of the formulation is composed of collagen fibers oriented parallel to the articular surface. In some forms, the articular chondrocytes in the superficial layer are elongated and flattened. In some embodiments, the articular chondrocytes in the superficial layer of the articular cartilage are tightly packed and aligned parallel to the articular surface. In some forms, the superficial layer of the articular cartilage expresses proteoglycans and other ECM genes unique to the superficial layer of cartilage (e.g., PRG4/lubricin, COL1A1).

In some embodiments, the intermediate zone of the formulation comprises collagen fibers. In some forms, the collagen fibers are thick, less organized, and are typically in an oblique orientation to the articular surface. In some forms, the repertoire of proteoglycans and other ECM genes expressed in the intermediate zone of the formulation is different from the superficial zone (e.g., higher COL2A1, CNMD). In some forms, the collagen fibers are in a perpendicular orientation to the articular surface. In some forms, articular chondrocyte morphology in the intermediate zone is more rounded than the flattened chondrocytes of the superficial zone.

In some embodiments the articular cartilage tissue has biomechanical properties that are similar to native cartilage tissue. The biomechanical properties are measured in an indentation assay as well as an unconfined compression using 3 to 4 stress/relaxation phases (0-5%, 5-10%, 10-15%, 15-20%). The equilibrium modulus (E) of micromass derived articular cartilage tissues ranges from 60-2000 kPa, preferably between 200-600 kPa. An example of the modulus (E) of the biomaterial encapsulated cartilage tissue by indentation is 1.68-1.91 MPa.

In some embodiments, the ECM of the articular cartilage is a hyperhydrated tissue. The percentage of water in the ECM can range from about 60% to about 95% of the total wet weight of the ECM. The ECM also includes the macromolecular proteins: type II collagen, and the large highly negatively charged proteoglycan, aggrecan. In some embodiments, several other classes of molecules, including lipids, phospholipids, proteins, and glycoproteins, also make up a portion of the ECM. Type II collagen is the major structural protein of ECM and is the major fibrillar collagen of articular cartilage and constitutes 90% to 95% of total collagen and 10% of the wet weight of articular cartilage. Type IX and XI collagen are also present.

### III. Methods of Generating Chondrocytes and Cartilage Compositions

Methods of generating chondrocytes and cartilage tissues involve inducing human pluripotent stem cell (hPSC)-derived progenitors into chondrogenic differentiation, thereby providing chondrocytes with desirable features for use in cartilage tissue engineering. Prior methods of generating chondrocytes from hPSC are described in detail in U.S. Pat. Nos. 9,993,504 and 10,736,923, which disclose a three-stage process for obtaining articular chondrocytes from hPSCs. By contrast, the disclosed methods, in some embodiments, use a four-stage process from hPSCs to articular chondrocytes, and different combination of chemical inducers to induce conversion of intermediate paraxial mesoderm cells plated at a low density (monolayer), into articular chondrocytes In some embodiments, methods disclosed herein include the activation of the TGFβ pathway in hPSC-derived chondrogenic progenitors promotes the efficient development of articular chondrocytes that can form stable cartilage tissue in vitro and in vivo.

Typically, the methods of differentiation of paraxial mesoderm, chondrocyte progenitors, and cartilage tissues from human pluripotent stem cells (hPSCs) include one or more of the following steps:
(a) induction of a primitive streak-like mesoderm population from hPSCs (Stage I),
(b) generating a paraxial mesoderm population from a primitive streak-like mesoderm population (Stage II),
(c) generating a chondrocyte precursor population from a paraxial mesoderm population (Stage III), via micromass (high cell density) culture, and
(d) generating articular chondrocytes (Stage IV) from a chondrocyte precursor population via micromass (high cell density) culture; or
in another embodiment,
(e) generating chondrogenic progenitors (Stage III) from a paraxial mesoderm population via a low density (monolayer) culture.

High cell density is used herein to refer to plating about 200,000 cells-about 1,000,000 cells per about 0.2 cm-about 2 cm diameter surface area (2D), or with respect to micromass is at least about 100,000 cells per about 20 microliters of media, or for example up to about 2,000,000 cells per about 20 microliters of media to allow for cells to adhere to the small surface area permitted for a micromass ‘spot’. For membrane filters, the area is dependent on the commercially available membrane that is purchased and the volume used to dictate biomaterial (if used) thickness, for example approximately 400,000 cells-about 5,000,000 cells can be plated in about 100 microliters-about 500 microliters of media or biomaterial in for example about 0.5 cm-about 2 cm diameter cylinder-shaped membrane filter-containing insert to allow cells to adhere. In both micromass and membrane filter culture, cells adhere in about a 1-5 cell layer and tissue is permitted to grow ‘thicker’ after adherence. A similar cell density could be used to seed onto a bone matrix or a bone substitute scaffold such as calcium polyphosphate (CPP).

A cell culture is referred to as a ‘monolayer culture’ herein, when cell density varies between 20×10³ per cm² and 20×10⁴ per cm². Beyond this cell concentration, the culture can be defined as high-density culture, which has characteristics very different from those of a monolayer.

The cells are generally subjected to adherent cell culture. The substrate for the adherent culture may be any one or combination of tissue culture treated plastic, polyorithine, laminin, poly-lysine, purified collagen, gelatin, fibronectin, tenascin, vitronectin, entactin, heparin sulfate proteoglycans, poly glycolytic acid (PGA), poly lactic acid (PLA), and poly lactic-glycolic acid (PLGA). In one embodiment, the cells are plated on MATRIGEL®-coated plates. In another embodiment, the cells are plated on fibronectin-coated plates. Cells can be cultured in filter cultures and micromass cultures. In an embodiment, cells are plated onto membrane filters, optionally those that are placed into tissue cultures dishes as part of a transwell system (e.g., MILLIPORE®, ALVATEX®). The substrate could also be a bone scaffold substitute such as CPP (calcium polyphosphate) or other pharmaceutically available scaffolds available.

Micromass culture is when a high-density suspension of cells is permitted to adhere in a single cell layer to a small area of the substrate (e.g., 200,000-500,000 cells adhere to a 0.2-1 cm diameter circular area of the substrate). Any shape or size of substrate can be used, prepared for example by 3D printing. The term “suspension” as used in the context of cell culturing is used as it is in the art. Namely, cell culture suspensions are cell culture environments where the cells are not adherent to a surface. One of skill in the art will be familiar with cell culture techniques, including, but not limited to, the use of equipment such as flow hoods, incubators and/or equipment used to keep the cells in constant motion, e.g., rotator platforms, shakers, etc., if necessary.

### A. Generating a Primitive Streak-Like Mesoderm Population (Stage I)

Generally, any human pluripotent stem cell population can be used as the starting population, including induced pluripotent stem cell populations. In one embodiment, the starting population is a human embryonic stem cell population (hESC) or an induced pluripotent stem cell population (iPSCs), optionally, primary hESC and/or primary iPSC. Many human ESC lines are commercially available and listed, for example, on the NIH HESC registry. In one embodiment, the human ESC population is a cell line optionally selected from a HES2, H1, H9, or any NIH ESC Registry available hESC cell line, or any human iPS cell line, such as any commercially available iPS cell lines, for example, as available from System Biosciences.

In some embodiments, the pluripotent cell population is contacted with the primitive streak inducing cocktail for between about 1 to about 5 days. In one embodiment, the primitive streak inducing cocktail includes an activin agonist, such as activin A or nodal; a BMP4 agonist, such as BMP4, BMP2, BMP6, BMP7 and/or, BMP10; and a FGF agonist, such as bFGF, FGF2, FGF4, FGF9 and/or optionally FGF 19, 21, 3, 5, 6, 8a, 16-18, 20 and/or 23, preferably, by culturing the cells in cell culture medium supplemented with effective amounts of these factors.

In some embodiments, the cell culture medium is also supplemented with a Wnt agonist. In some embodiments, Stage I does not include a step that results in the formation of embryoid bodies. In another embodiment, the initial stage of differentiation involves the induction of a primitive streak-like mesoderm population by contacting the pluripotent cells with a primitive streak inducing cocktail including Activin A, BMP4 and basic FGF from days 1 to 4 of differentiation. Typically, on day 3, mesoderm populations are monitored by the expression of CD56 and PDGFRα on the cell surface by flow cytometry. In some embodiments, the contacting with the primitive streak inducing cocktail is shortened, for example, from days 1-3, if the CD56+/PDGFRα+population is generated sooner.

Brachyury expression is also induced during this stage, as monitored by gene expression on approximately day 2-3, and the expression of cell surface markers PDGFRα and CD56 by day 4. In human PSCs, PS-like mesoderm induction relies on activin and Wnt signaling and is monitored by Brachyury and PDGFRα expression.

In some embodiments, CD56 is used to monitor for example human primitive streak cell formation. The appearance of cell surface markers such as CD56 and PDGFRα indicates that a primitive streak-like mesoderm population has been generated. In some embodiments, the pluripotent cell population is induced with a primitive streak inducing cocktail for one day, 2 days, 3 days, 4 days, or 5 days.

### 1. Feeder-Free Monolayer Culture

In some embodiments, the hPSCs are cultured on irradiated mouse fibroblasts/feeders (MEFs) and knockout serum-based media (KSR). In preferred embodiments, the hPSCs are cultured on feeder-free culture.

The maintenance of undifferentiated human pluripotent stem cells (hPSC) under xeno-free condition requires the use of human feeder cells or extracellular matrix (ECM) coating. However, human-derived sources may cause human pathogen contamination by viral or non-viral agents to the patients. A feeder-free culture system is designed to keep the stem cells from differentiating while protecting the stem cells from direct contact with the feeder in an effort to prevent cross-contamination or passing nonhuman pathogens into the stem cells.

In some embodiments, the pluripotent cells are cultured as embryoid bodies. In other embodiments, the pluripotent stem cells are cultured as a monolayer. Human embryonic stem cells (hESCs) are often cocultured on mitotically inactive fibroblast feeder cells to maintain their undifferentiated state. Under these growth conditions, hESCs form multilayered colonies of morphologically heterogeneous cells surrounded by flattened mesenchymal cells. In contrast, hPSC grown in feeder cell-conditioned medium on Matrigel or other ECM coating, instead tend to grow as monolayers with uniform morphology.

### 2. Addition of a Wnt Agonist

Blocking Wnt signaling with an antagonist inhibits primitive streak formation. In some embodiments, a Wnt agonist is added to an hPSC line to enhance the development of a CD56+PDGFRa+ primitive streak-like population, for example, added from day 1 to day 3.

In cell lines and starting populations where endogenous Wnt signaling is absent or low, adding a Wnt agonist improves the efficiency of primitive streak formation from PSCs. Thus, in preferred embodiments, the primitive streak inducing cocktail further comprises a Wnt agonist. Exemplary Wnt agonist is Wnt3a or a GSK-3 selective inhibitor such as CHIR-99021 (STEMOLECULE™ CHIR99021 Stemgent), 6-Bromolndirubin-3′-Oxime (BIO) (Cayman Chemical (cat:13123)), or STEMOLECULE™ BIO from Stemgent (cat:04003).

Endogenous Wnt signaling is sufficient for primitive streak induction in some cell lines e.g., HES2, H9. Thus, in other embodiments, no external Wnt agonist is added to an hPSC line to develop into a primitive streak population.

### B. Generating a Paraxial Mesoderm Population

In some embodiments, the methods include the step of culturing a primitive streak-like mesoderm population to generate a paraxial mesoderm population. In some embodiments, the primitive streak-like mesoderm population is a CD56+, PDGFRα+ primitive streak-like mesoderm population. In some embodiments, the primitive streak-like mesoderm population is cultured with a paraxial mesoderm specifying cocktail including:
(i) a FGF agonist;
(ii) a BMP inhibitor, such as Noggin, LDN-193189, and/or Dorsomorphin; and
(iii) optionally one or more of a TGFβ inhibitor, optionally SB431542; and a Wnt inhibitor, optionally IWP2 (N-(6-Methyl-2-benzothiazolyl)-2-[(3,4,6,7-tetrahydro-4-oxo-3-phenylthieno[3,2-d]pyrimidin-2-yl)thio]-acetamide; Sigma); Dickkopf-related protein 1 (DKK1; R & D Systems), and/or XAV939 (3,5,7,8-Tetrahydro-2-[4-(trifluoromethyl)phenyl]-4H-thiopyrano[4,3-d]pyrimidin-4-one; Sigma);

In some embodiments, the generation of paraxial mesoderm is generally characterized by the expression of transcription factors Meox1 and Nkx3.2. In preferred embodiments, the culturing condition induce to specify a paraxial mesoderm population expressing one or more of cell surface molecules of CD73, CD105 and/or PDGFR3. In some embodiments, the paraxial mesoderm population expressing CD73+CD105+, or CD73+PDGFRβ, or CD73+CD105+PDGFRβ+.

In some embodiments, the primitive streak (PS)-like cells are induced to a paraxial fate in monolayer culture during this stage (e.g., day 3-15). In some embodiments, BMP signaling is inhibited in the primitive streak (PS)-like cells using a molecule such as Dorsomorphin, and TGFβ signaling is inhibited using a small molecule such as SB431542. Human paraxial mesoderm requires the addition of FGF (such as bFGF) and it is added to culture media for example, between days 3 and 15 in monolayer culture. Data suggest that Wnt inhibition is better for cartilage potential later, and without Wnt inhibition more tendon/ligament gene expression was observed. Thus, in preferred embodiments, a Wnt antagonist is also added. Human paraxial mesoderm is determined by the expression of cell surface markers including CD73, CD105, and PDGFRβ.

In one embodiment, human paraxial mesoderm is specified with the BMP inhibitor Dorsomorphin (e.g., days 3-5) and FGF during a monolayer culture between days 3 and 14 of differentiation. Human paraxial mesoderm is characterized by the expression of cell surface markers CD73, CD105, PDGFRβ, and/or, Meox1 and Nkx3.2 gene expression on day 14. Typically, expression of these markers begins at or around day 11 and is maximal at or around day 14.

### 1. Timing of Paraxial Mesoderm Specification

In some embodiments, FGF treatment persists for 11 days to get ‘Paraxial mesoderm’ on day 14. In some embodiments, the culturing to produce paraxial mesoderm population is about 1, 2, 3, 4, 5, 6, 7, 8, 9, 10, and 11 days. In preferred embodiments, the time required to produce paraxial mesoderm population is about 1, 2, 3, 4, 5, or less than 5 days, less than 6 days, less than 7 days, less than 8 days, less than 9 days, less than 10 days.

### C. Generating a Chondrocyte Precursor Population

In some embodiments, the methods include the step of culturing the paraxial mesoderm population to generate a chondrocyte precursor population. In some embodiments, the paraxial mesoderm population expressing CD73, CD105 and/or PDGFR-β is cultured at a high cell density optionally in serum free or serum containing media. In other embodiments, the paraxial mesoderm population expressing CD73, CD105 and/or PDGFR-β is cultured at a low cell density such as in monolayer, optionally in serum free or serum containing media.

The paraxial mesoderm population expressing CD73+, CD105+ and/or PDGFRD+ is cultured with a TGF-β agonist such as TGFβ 3 in serum free media at high density to produce a SOX9+, COL2A1+chondrocyte/chondrocyte precursor population.

### D. Generating a Differentiated Chondrocytes and Cartilage Tissue

### 1. Generating Differentiated Chondrocytes Via Micromass Culture of Chondrocyte Precursor Cells

In some embodiments, the methods include the step of further culturing the chondrocyte precursor population to generate a cartilage tissue. In this embodiment, the high cell density SOX9+, COL2A1+ chondrocyte precursor population is cultured with the TGFβ3 agonist for a period of time effective to produce an articular like non-hypertrophic chondrocyte cells and/or cartilage like tissue. In other embodiments, the high cell density SOX9+, COL2A1+ chondrocyte precursor population is cultured with a BMP4 agonist for a period of time effective to produce a hypertrophic chondrocyte like cells and/or cartilage like tissue.

In one embodiment, the paraxial mesoderm population is cultured as high-density micromass in tissue culture medium including a combination of agents of a TGFβ agonist, an FGF agonist, and a cyclic AMP agonist.

### 2. Generating Differentiated Chondrocytes Via Monolayer Culture of Paraxial Mesoderm

In some embodiments, the methods include the step of generating chondrogenic precursors directly from the paraxial mesoderm population in low-density monolayer culture, and subsequent cartilage tissues are generated from these chondrogenic precursors.

### Method for Generating Chondrogenic Precursor in Low Density Monolayer

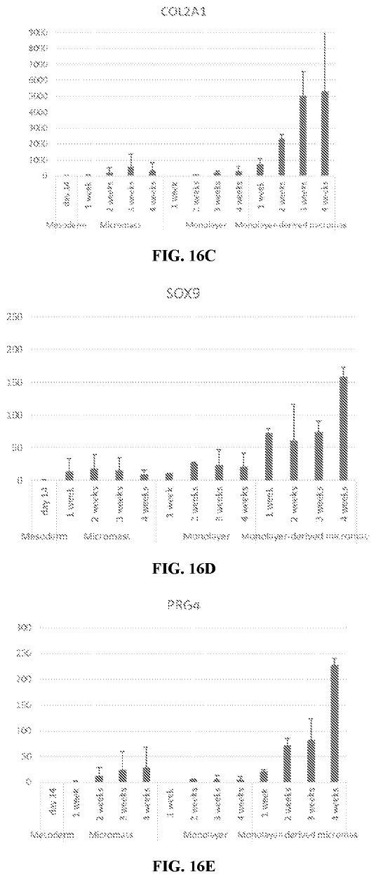

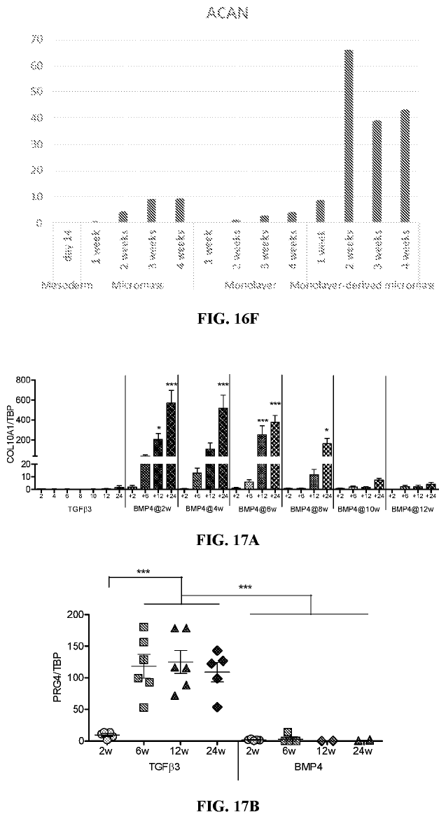

Experiments in the Examples below have shown that Forskolin increases chondrogenic potential of monolayer cells treated with TGFβ agonist and FGF agonist, and in cells cultured without TGFβ agonist or FGF agonist. Chondrocytes derived in monolayer with FGF treatment only (no exogenous TGFβ) or FGF with exogenous TGFβ with or without Forskolin were suitable for use in cartilage tissue engineering. The advantages of deriving chondrocytes in monolayer over micromass cultures include but are not limited to (1) a higher cell yield, (2) greater chondrocyte viability, and (3) reduced generation time of the articular cartilage (e.g., about 3 weeks to generate articular cartilage from monolayer-derived chondrocytes compared to at least 6-12 weeks to generate articular cartilage tissues in direct micromass cultures). Thus, in some embodiments, the chondrocytes derived in monolayer of the paraxial mesoderm cells have a 10%, 20%, 30%, 40%, 50%, 60%, 70%, 80%, 90%, 100%, or over 100% more viable cells, preferably chondrocyte cells expressing one or more of SOX9, COL2A1, ACAN, and PRG4, more than those derived from a high-density culture of the paraxial mesoderm cells, provided they have the same number of paraxial mesoderm cells as a starting population (FIG. 16D-16F).

Thus, in some embodiments, the paraxial mesoderm population expressing CD73+, CD105+ and/or PDGFRβ+ is cultured with one or more of FGF agonist, cAMP agonist, and TGFβ agonist. Exemplary FGF agonists include basic FGF or FGF2. Additional FGF include FGF4, FGF9, FGF 19, 21, 3, 5, 6, 8a, 16-18, 20, and 23. Exemplary cAMP agonists include Forskolin, 8-bromo-cAMP, and colforsin. Exemplary TGFβ agonists include TGFβ 1, TGFβ2, TGFβ3. In one embodiment, the paraxial mesoderm population expressing CD73+, CD105+ and/or PDGFRβ+ is cultured with FGF agonist, Forskolin, and TGFβ agonist. In another embodiment, the paraxial mesoderm population expressing CD73+, CD105+ and/or PDGFRβ+ is cultured with Forskolin alone without FGF agonist or TGFβ agonist. In a further embodiment, the paraxial mesoderm population expressing CD73+, CD105+ and/or PDGFRβ+ is cultured with FGF agonist alone without cAMP agonist or TGFβ agonist.

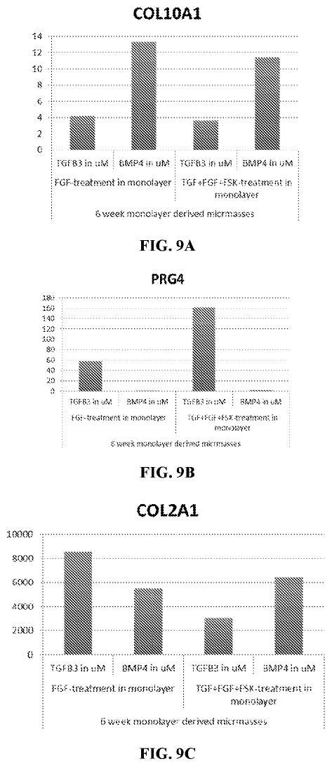

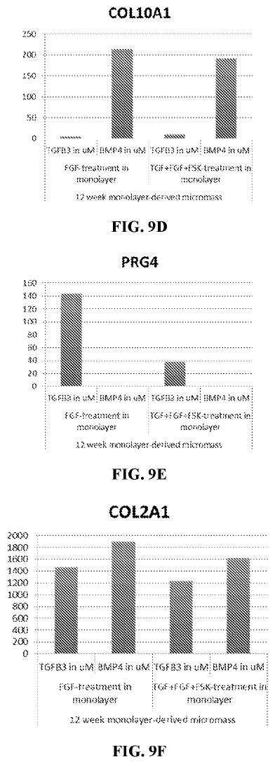

In preferred embodiments, chondrocytes derived in monolayer with any of the above conditions are further used for cartilage tissue engineering. Cartilage tissue of desirable dimensions, composition can be subsequently produced via micromass or encapsulation in biomaterials for implantation. For example, FIGS. 8A-8D and 9A-9F illustrate results from experiments conducted in an exemplary embodiment. FIG. 8A-D are bar graphs showing results from qPCR experiments comparing the expression of genes associated with encapsulated cartilage tissues (COL2A1 and PRG4) grown from either monolayer-derived or micromass-derived chondrocytes. FIG. 9A-9F shows qPCR results of the expression of cartilage-associated genes in micromass tissues derived from the monolayer-derived chondrocytes as disclosed herein. Chondrocyte progenitors derived in monolayer with either FGF alone or TGFB+FGF+FSK can generate articular and growth plate cartilage later in micromass.

In preferred embodiments, chondrocytes derived in monolayer with any of the above conditions provide articular chondrocytes tissues having abundant sulfated glycosaminoglycans (sGAGs), and/or other components of the extracellular matrix, more preferably at a level comparable to range within human articular cartilage.

### IV. Methods of Culturing and Expanding Chondrocytes In Vitro

Improved methods of serially generating and expanding chondrocytes in vitro are also described. In some embodiments, micromass-derived chondrocytes expanded in serum-containing or serum-free media retained ability to make cartilage in new micromass or when encapsulated in biomaterial after passaging. In other embodiments, micromass-derived chondrocytes expanded and passaged in 2% serum media, in serum-free media with TGFβ and FGF, or in serum-containing or serum-free media, in serum-free media with FGF, in serum-free media with TGFβ, FGF, and FSK, for up to 3 passages and retained ability to make cartilage tissue in new micromass or when encapsulated in biomaterial after passaging. In some embodiments, the micromass has been cultured for at least 2, 3, 4, 5, 6, 7, 8, 9, 10, 11, 12 weeks prior to serial replating and/or expansion.

In some embodiments, micromass-derived chondrocytes are replated and/or expanded for at least 1, 2, 3, 4, 5, 6, 7, 8, 9, 10 passages prior to generating cartilage tissues suitable for transplant. In preferred embodiments, the cells after replating and/or expanding are responsive to TGFβ for articular cartilage and/or BMP for growth plate cartilage throughout all passages. In further preferred embodiments, micromass-derived chondrocytes are replated and/or expanded in serum-free media. In some embodiments, micromass-derived chondrocytes are replated in monolayer or into new micromasses (high density cell culture). In some embodiments, the micromass-derived chondrocytes after replating and/or expanding produce new cartilage tissues smaller in size compared to those with less or with no replating and/or expanding in monolayer.

Methods: Monolayer-derived and micromass-derived chondrocytes are enzymatically isolated from the encompassing extracellular matrix prior to passage or cryopreservation, typically mediated by collagenase. Approximately between 500,000 and 5 million cells are plated per well on 6-well plates or 10 cm dishes coated with gelatin, type II collagen, or fibronectin, with culture condition of DMEM (plus ITS/proline/Dexamethasone)+2% FBS+Ascorbic acid or serum-free media (SFD) supplemented with a combination of FGF, TGFB, FSK or no supplements. Cells were cultured until confluent (2-3 cell doublings) (˜2-5 days), and then trypsinized and replated at similar density.

### V. Methods of Encapsulating Chondrocytes for Tissue Engineering

Examples of cartilage tissue implants/constructs were created by encapsulating hPSC-derived articular chondrocytes in RAD16-I, a self-assembling peptide scaffold commercially known as Puramatrix (Corning), to a final concentration of 0.01% to 0.5%, preferably 0.15% RAD16-I. This biomaterial is polymerized by increases in pH via graduated equilibration with culture media over a period of several minutes to 2 hours.

Puramatrix has repeating units of hydrophilic-hydrophobic amino acids. In ionic or neutral environments, it spontaneously self-assembles in antiparallel β-sheet configuration, generating an interweaving network of nanofibers (pores 50-200 nm). Its non-covalent interactions allow cell migration. Stiffness can be controlled by varying concentration (100 Pa-6 kPa).

In some forms, hPSC-derived articular chondrocytes may also be encapsulated in biomaterials comprised of combinations of hyaluronic acid, chondroitin sulfate, and collagens (primarily collagen I), and cartilage tissues resulted with similar success.

In some embodiments, the chondrocytes are encapsulated in hydrogels as tissue engineering scaffolds. Hydrogels can be polymerized using light, UV radiation, a redox agent (e.g., sodium thiosulfate in combination with sodium persulfate), changes in pH or by using some other suitable polymerization initiator such as a divalent cation like calcium.

The polymerizable agent may comprise monomers, macromers, oligomers, polymers, or a mixture thereof. The polymer compositions can consist solely of covalently cross-linkable polymers, or blends of covalently and ionically cross-linkable or hydrophilic polymers.

Suitable hydrophilic polymers include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as FICOLL™, polysucrose, hyaluronic acid, dextran, heparan sulfate, chondroitin sulfate, heparin, or alginate, and proteins such as gelatin, collagen, albumin, or ovalbumin or copolymers or blends thereof. “celluloses” includes cellulose and derivatives of the types described above; “dextran” includes dextran and similar derivatives thereof.

Examples of materials that can be used to form a hydrogel include modified alginates. Alginate is a carbohydrate polymer isolated from seaweed, which can be crosslinked to form a hydrogel by exposure to a divalent cation such as calcium. Alginate is ionically crosslinked in the presence of divalent cations, in water, at room temperature, to form a hydrogel matrix. Modified alginate derivatives may be synthesized which have an improved ability to form hydrogels. The use of alginate as the starting material is advantageous because it is available from more than one source and is available in good purity and characterization. The term “modified alginates” refers to chemically modified alginates with modified hydrogel properties.

Naturally occurring alginate may be chemically modified to produce alginate polymer derivatives that degrade more quickly. For example, alginate may be chemically cleaved to produce smaller blocks of gellable oligosaccharide blocks and a linear copolymer may be formed with another preselected moiety, e.g. lactic acid or epsilon-caprolactone. The resulting polymer includes alginate blocks which permit ionically catalyzed gelling, and oligoester blocks which produce more rapid degradation depending on the synthetic design. Alternatively, alginate polymers may be used wherein the ratio of mannuronic acid to guluronic acid does not produce a film gel, which are derivatized with hydrophobic, water-labile chains, e.g., oligomers of epsilon-caprolactone. The hydrophobic interactions induce gelation, until they degrade in the body.

Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Polysaccharides which gel in the presence of monovalent cations form hydrogels upon exposure, for example, to a solution comprising physiological levels of sodium. Hydrogel precursor solutions also may be osmotically adjusted with an anion, such as mannitol, and then injected to form a gel.

Polysaccharides that are very viscous liquids or are thixotropic and form a gel overtime by the slow evolution of structure, are also useful. For example, hyaluronic acid, which forms an injectable gel with a consistency like a hair gel, may be utilized. Modified hyaluronic acid derivatives are particularly useful. The term “hyaluronic acids” refers to natural and chemically modified hyaluronic acids. Modified hyaluronic acids may be designed and synthesized with preselected chemical modifications to adjust the rate and degree of crosslinking and biodegradation. For example, modified hyaluronic acids may be designed and synthesized which are esterified with a relatively hydrophobic group such as propionic acid or benzylic acid to render the polymer more hydrophobic and gel-forming, or which are grafted with amines to promote electrostatic self-assembly. Modified hyaluronic acids thus may be synthesized which are injectable, in that they flow under stress, but maintain a gel-like structure when not under stress.

Other materials which may be utilized include proteins such as fibrin, collagen, and gelatin. Other polymeric hydrogel precursors include polyethylene oxide-polypropylene glycol block copolymers such as PLURONICS™ or TETRONICS™, which are crosslinked by hydrogen bonding and/or by a temperature change, as described in Steinleitner et al., Obstetrics & Gynecology, 77:48-52 (1991); and Steinleitner et al., Fertility and Sterility, 57:305-308 (1992). Polymer mixtures also may be utilized. For example, a mixture of polyethylene oxide and polyacrylic acid which gels by hydrogen bonding upon mixing may be utilized. In one embodiment, a mixture of a 5% w/w solution of polyacrylic acid with a 5% w/w polyethylene oxide (polyethylene glycol, polyoxyethylene) 100,000 can be combined to form a gel over the course of time, e.g., as quickly as within a few seconds.

Water soluble polymers with charged side groups may be crosslinked by reacting the polymer with an aqueous solution containing ions of the opposite charge, either cations if the polymer has acidic side groups or anions if the polymer has basic side groups. Examples of cations for cross-linking of the polymers with acidic side groups to form a hydrogel are monovalent cations such as sodium, divalent cations such as calcium, and multivalent cations such as copper, calcium, aluminum, magnesium, strontium, barium, and tin, and di-, tri- or tetra-functional organic cations such as alkylammonium salts. Aqueous solutions of the salts of these cations are added to the polymers to form soft, highly swollen hydrogels and membranes. The higher the concentration of cation, or the higher the valence, the greater the degree of cross-linking of the polymer. Additionally, the polymers may be crosslinked enzymatically, e.g., fibrin with thrombin.

Suitable ionically crosslinkable groups include phenols, amines, imines, amides, carboxylic acids, sulfonic acids and phosphate groups. Aliphatic hydroxy groups are not considered to be reactive groups for the chemistry disclosed herein. Negatively charged groups, such as carboxylate, sulfonate and phosphate ions, can be crosslinked with cations such as calcium ions. The crosslinking of alginate with calcium ions is an example of this type of ionic crosslinking. Positively charged groups, such as ammonium ions, can be crosslinked with negatively charged ions such as carboxylate, sulfonate and phosphate ions. Preferably, the negatively charged ions contain more than one carboxylate, sulfonate, or phosphate group.

The preferred anions for cross-linking of the polymers to form a hydrogel are monovalent, divalent or trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a semi-permeable surface membrane. Examples of materials that can be used include polymers having basic reactive groups such as amine or imine groups, having a preferred molecular weight between 3,000 and 100,000, such as polyethylenimine and polylysine. These are commercially available. One polycation is poly(L-lysine); examples of synthetic polyamines are: polyethyleneimine, poly(vinylamine), and poly(allyl amine). There are also natural polycations such as the polysaccharide, chitosan.

Polyanions that can be used to form a semi-permeable membrane by reaction with basic surface groups on the polymer hydrogel include polymers and copolymers of acrylic acid, methacrylic acid, and other derivatives of acrylic acid, polymers with pendant SO3H groups such as sulfonated polystyrene, and polystyrene with carboxylic acid groups. These polymers can be modified to contain active species polymerizable groups and/or ionically crosslinkable groups. Methods for modifying hydrophilic polymers to include these groups are well known to those of skill in the art.

The polymers may be intrinsically biodegradable but are preferably of low biodegradability (for predictability of dissolution) but of sufficiently low molecular weight to allow excretion. The maximum molecular weight to allow excretion in human beings (or other species in which use is intended) will vary with polymer type but will often be about 20,000 Daltons or below. Usable, but less preferable for general use because of intrinsic biodegradability, are water-soluble natural polymers and synthetic equivalents or derivatives, including polypeptides, polynucleotides, and degradable polysaccharides.

The polymers can be a single block with a molecular weight of at least 600, preferably 2000 or more, and more preferably at least 3000. Alternatively, the polymers can include can be two or more water-soluble blocks which are joined by other groups. Such joining groups can include biodegradable linkages, polymerizable linkages, or both. For example, an unsaturated dicarboxylic acid, such as maleic, fumaric, or aconitic acid, can be esterified with hydrophilic polymers containing hydroxy groups, such as polyethylene glycols, or amidated with hydrophilic polymers containing amine groups, such as poloxamines.

Covalently crosslinkable hydrogel precursors also are useful. For example, a water-soluble polyamine, such as chitosan, can be cross-linked with a water soluble diisothiocyanate, such as polyethylene glycol diisothiocyanate. The isothiocyanates will react with the amines to form a chemically crosslinked gel. Aldehyde reactions with amines, e.g., with polyethylene glycol dialdehyde also may be utilized. A hydroxylated water-soluble polymer also may be utilized.

Alternatively, polymers may be utilized which include substituents which are crosslinked by a radical reaction upon contact with a radical initiator. For example, polymers including ethylenically unsaturated groups which can be photochemically crosslinked may be utilized. In this embodiment, water soluble macromers that include at least one water soluble region, a biodegradable region, and at least two free radical-polymerizable regions, are provided. The macromers are polymerized by exposure of the polymerizable regions to free radicals generated, for example, by photosensitive chemicals and or light. Examples of these macromers are PEG-oligolactyl-acrylates, wherein the acrylate groups are polymerized using radical initiating systems, such as an eosin dye, or by brief exposure to ultraviolet or visible light. Additionally, water soluble polymers which include cinnamoyl groups which may be photochemically crosslinked may be utilized, as disclosed in Matsuda et al., ASAID Trans., 38:154-157 (1992).

In general, the polymers are at least partially soluble in aqueous solutions, such as water, buffered salt solutions, or aqueous alcohol solutions. Methods for the synthesis of the other polymers described above are known to those skilled in the art. See, for example Concise Encyclopedia of Polymer Science and Polymeric Amines and Ammonium Salts, E. Goethals, editor (Pergamen Press, Elmsford, N.Y. 1980). Many polymers, such as poly(acrylic acid), are commercially available. Naturally occurring and synthetic polymers may be modified using chemical reactions available in the art and described, for example, in March, “Advanced Organic Chemistry,” 4^{th} Edition, 1992, Wiley-Interscience Publication, New York.

Preferably, the hydrophilic polymers that include active species or crosslinkable groups include at least 1.02 polymerizable or crosslinkable groups on average, and, more preferably, each includes two or more polymerizable or crosslinkable groups on average. Because each polymerizable group will polymerize into a chain, crosslinked hydrogels can be produced using only slightly more than one reactive group per polymer (i.e., about 1.02 polymerizable groups on average). However, higher percentages are preferable, and excellent gels can be obtained in polymer mixtures in which most or all of the molecules have two or more reactive double bonds. Poloxamines, an example of a hydrophilic polymer, have four arms and thus may readily be modified to include four polymerizable groups.

The hydrogel solution is prepared, for example, by mixing 10% weight/volume (w/v) of the polymerizable polymer in sterile phosphate buffered saline (PBS), which is a suitable solvent, adjusted to a pH of about 7.4. In some embodiments, the polymer is either photopolymerizable poly(ethylene glycol) diacrylate (PEGDA) or photopolymerizable poly(ethylene oxide) diacrylate (PEODA).

Optionally, various additives can be included in the hydrogel solution such as 100 U/ml of penicillin and 100 μg/ml streptomycin to inhibit microbial contamination. However, these are not the only bioactive additives that can be included in the hydrogel solution. For example, the bioactive additives could include, singly or in combination, growth factors, cell differentiation factors, other cellular mediators, nutrients, antibiotics, anti-inflammatories, and other pharmaceuticals. Although not limiting, some suitable cellular growth factors, depending upon the cell type to be encapsulated in either the hydrogel of the same or adjacent hydrogel layer, include heparin binding growth factor (HBGF), transforming growth factor (TGFα or TGFβ), alpha fibroblastic growth factor (FGF), epidermal growth factor (EGF), vascular endothelium growth factor (VEGF), various angiogenic factors, nerve growth factor (NGF) and muscle morphologic growth factor.

In addition, the hydrogel solution optionally includes a suitable non-toxic polymerization initiator, mixed thoroughly to make a final concentration of 0.05% w/v. When PEGDA or PEODA are selected as the polymers, the polymerization initiator is preferably added and selected to be the photoinitiator Igracure 2959 (commercially available from Ciba Specialty Chemicals Corp., Tarrytown, N.Y.), although other suitable photoinitiators can be used.

Exemplary photopolymerizable polymers are PEGDA and PEODA. Suitable hydrophilic polymers include synthetic polymers such as partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll® polysucrose, hyaluronic acid, dextran, heparan sulfate, chondroitin sulfate, heparin, or alginate, and proteins such as gelatin, collagen, albumin, or ovalbumin or copolymers or blends thereof. The term “celluloses” includes cellulose and derivatives of the types described above; “dextran” includes dextran and similar derivatives thereof.

Exemplary photoinitiator is Igracure 2959. Other photoinitiators include HPK, which is commercially available from Polysciences. In addition, various dyes and an amine catalyst are known to form an active species when exposed to external radiation. Specifically, light absorption by the dye causes the dye to assume a triplet state, which subsequently reacts with the amine to form the active species that initiates polymerization. Typically, polymerization can be initiated by irradiation with light at a wavelength of between about 200-700 nm, most preferably in the long wavelength ultraviolet range or visible range, 320 nm or higher, and most preferably between about 365 and 514 nm.

Numerous dyes can be used for photopolymerization, and these include erythrosin, phloxime, rose bengal, thonine, camphorquinone, ethyl eosin, eosin, methylene blue, riboflavin, 2,2-dimethyl-2-phenylacetophenone, 2-methoxy-2-phenylacetophenone, 2,2-dimethoxy-2-phenyl acetophenone, other acetophenone derivatives, and camphorquinone. Suitable cocatalysts include amines such as N-methyl diethanolamine, N,N-dimethyl benzylamine, triethanol amine, triethylamine, dibenzyl amine, N-benzylethanolamine, N-isopropyl benzylamine. Triethanolamine is a preferred cocatalyst with one of these dyes. Photopolymerization of these polymer solutions is based on the discovery that combinations of polymers and photoinitiators (in a concentration not toxic to the cells, less than 0.10% by weight, more preferably between 0.05 and 0.01% by weight percent initiator) will crosslink upon exposure to light equivalent to between one and 3 mWatts/cm2.

While photopolymers are preferred for making the hydrogels, because it is convenient to control polymerization using external radiation supplied through a surgical scope, the present invention can be practiced using other polymer materials and polymerization initiators. Examples of other materials which can be used to form a hydrogel include (a) modified alginates, (b) polysaccharides (e.g. gellan cum and carrageenans) which gel by exposure to monovalent cations, (c) polysaccharides (e.g., hyaluronic acid) that are very viscous liquids or are thiotropic and form a gel over time by the slow evolution of structure, and (d) polymeric hydrogel precursors (e.g., polyethylene oxide-polypropylene glycol block copolymers and proteins).

### VI. Methods of Cryopreserving Chondrocytes

Methods of cryopreservation of chondrocytes with high viability are known to those of skill in the art. In some embodiments, as described in Example 7, the hPSC-derived chondrocytes retain the ability to generate articular cartilage following freeze-thaw cycles following cryopreservation. As described herein, a cryopreservation solution is used for the cryopreservation of viable chondrocytes and subsequent generation of articular cartilage tissues after thawing.

Cryopreservation solution refers to any solution or media in which biological material (such as chondrocytes) is immersed before cryopreservation. Typically, cryopreservation solutions contain a balanced salt solution such as phosphate buffered saline and at least one cryoprotectant. Cryoprotectants are substances that reduce the damage incurred by the cells or tissues during freezing and/or thawing. Most freezing solutions are composed of intracellular cryoprotectants (e.g., DMSO, glycerol, ethylene glycol, polyethylene glycol, 1,2-propanediol, formamide) and/or extra cellular cryoprotectants (sugars, proteins, carbohydrates such as: Hydroxy Ethyl Starch, dextran, etc.), Some optional cryopreservation solutions do not comprise glycosaminoglycans.

Cryoprotectants or cryoprotective agents (CPAs) required to prevent any freezing damage to cells are well known in the art (see, for example, Fuller, Cryo. Lett. 2004; 25:375-388, the contents of which are incorporated by reference herein). DMSO is the most common cryoprotectants used in cryopreservation of MSCs. Therefore, in some embodiments, chondrocytes are frozen in one or more cryo-preservatives including DMSO.

In some embodiments, the biomass-derived chondrocytes or the monolayer-derived chondrocytes are frozen, for example, in cryopreservation solution, i.e., the presence of cryoprotectant.

In some embodiments, after thawing from being frozen and stored in cryoprotectants for a period of time the micromass-derived chondrocytes or the monolayer-derived chondrocytes retain high viability, for example 95%, 90%, 85%, 80%, 75%, 70%, 65%, 60%, 55%, 50% viable cells out of the total number of cells frozen. In preferred embodiments, after thawing from being frozen and stored in cryoprotectants for a period of time the biomass-derived chondrocytes or the monolayer-derived chondrocytes retain cartilage potential and are capable of generating new cartilage tissue.

Monolayer-derived and micromass-derived chondrocytes must be enzymatically isolated from the encompassing extracellular matrix prior to cryopreservation, typically mediated by collagenase. In some embodiments, chondrocytes are resuspended in cryopreservation media comprised of 50% serum, 40% IMDM and 10% DMSO at cell densities ranging from 1 million to 50 million cells per milliliter, preferably about 5-10 million cells per milliliter. In another embodiment, chondrocytes are resuspended in commercial cryopreservation media Cryostor-CS10. In some embodiments, thawed cells are immediately cultured in biomaterials or high-density micromass to produce cartilage tissues. In another embodiment, thawed cells are plated in monolayer and expanded 1, 2, or 3 times prior to encapsulation in biomaterials or cultured in high density micromass to produce cartilage tissues. Cells expanded in monolayer acquire a less-chondrogenic phenotype that is restored upon high density micromass plating or encapsulation.

### VII. Methods of Using Cartilage Tissues

The methods provide chondrocytes and/or cartilage tissues suitable for transplant.

The methods can be used to treat or prevent one or more disease or disorders, in a subject in need thereof. In some embodiments, the subject has osteoarthritis, osteochondritis dissecans, polychondritis, other chondropathies, or injuries or damages affecting the cartilage. Thus, in some embodiments, the disclosed compositions and methods of use thereof are able to ameliorate one or more symptoms and/or treat osteoarthritis, osteochondritis dissecans, polychondritis, other chondropathies, or injuries or damages affecting the cartilage.

Cartilage (hyaline cartilage or articular cartilage) is a 1-5 mm thin tissue that coats the boney surfaces inside joints, as well as forms other lubricating strong surfaces. It provides a very low friction articulation that ideally lasts a lifetime. Cartilage may be damaged through acute injury or degeneration over time. For example, osteoarthritis (OA) is a joint disorder that leads to thinning of cartilage and progressive joint damage. Focal lesions of articular cartilage can progress to more widespread cartilage destruction and arthritis that is disabling. In some embodiments, the methods ameliorating one or more symptoms of cartilage damage, injury, and/or defects.

The chondrocytes and cartilage tissues prepared thereof can, in principle, be applied to any site in need of cartilage repair. Preferably, the disclosed cartilage tissues such as articular cartilage tissues, maybe in the form of chondral (osteochondral) auto- and allografts, prepared from the chondrocytes are fully functional cartilage tissues suitable for implanting into defects and more preferably for integration to the surrounding cartilage tissue. The disclosed cartilage tissues are particularly suited for implantation in vivo.

In some embodiments, the chondrocytes and cartilage tissues are administered or implanted to promote resurfacing, repair, and/or regeneration of cartilage. In preferred embodiments, the percentage of cartilages repaired and/or regenerated is about 5%, 10%, 20%, 30%, 40%, 50%, 60%, 70%, 80%, 90%, or more than 90% of the initially damaged, worn cartilage, in volume.

Methods of using the cartilage tissues or tissue implants to repair cartilage damage or defect are described. In some embodiments, the methods and compositions are effective in repairing and/or regenerating surface cartilage such as articular cartilage. In further embodiments, the cartilage tissues are implanted to repair articular cartilage in the femoral, tibial, and/or patellar articular surfaces to about 10%, 20%, 30%, 40%, 50%, 60%, 70%, 80%, 90%, 100%, 150%, 200%, 250%, 300%, or more than 300% of the damaged or worn cartilage present at the time of treatment, measured by cartilage volume.

The present invention will be further understood by reference to the following non-limiting examples.

### EXAMPLES

### Example 1: Serial Replating and Expansion/Replating of 12-Week-Old Micromass-Derived Chondrocytes

### Materials and Methods

### Expansion in Monolayer Culture

Chondrocytes within articular cartilage tissues (12-week-old micromass tissues) were dissociated from their matrix and immediately replated in micromass culture or expanded in monolayer briefly prior to replating in micromass culture. For monolayer expansion, approximately 2 million cells were plated per well on 10 cm dishes coated with gelatin with culture condition of DMEM (plus ITS/proline/Dex)+2% FBS+Ascorbic acid. Expanded cells were cultured until confluent (˜3 days), and then trypsinized and replated in additional monolayer expansion or plated into micromass after the first passage.

### Results

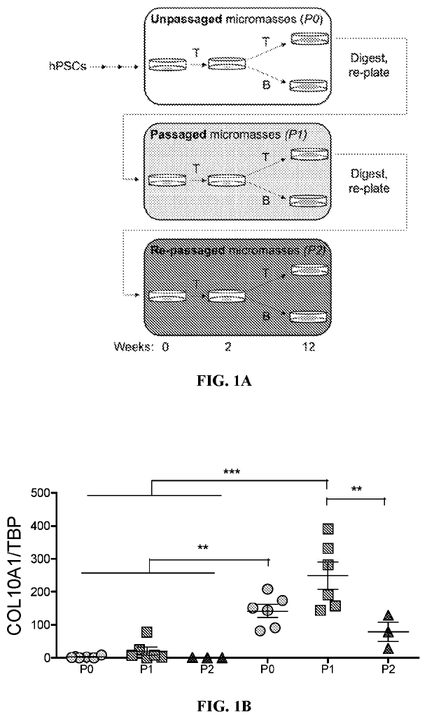

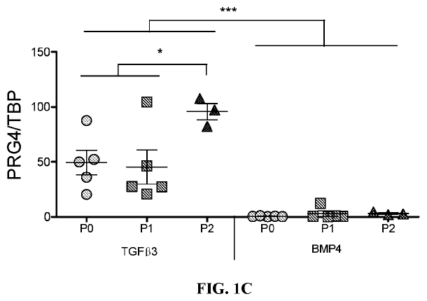

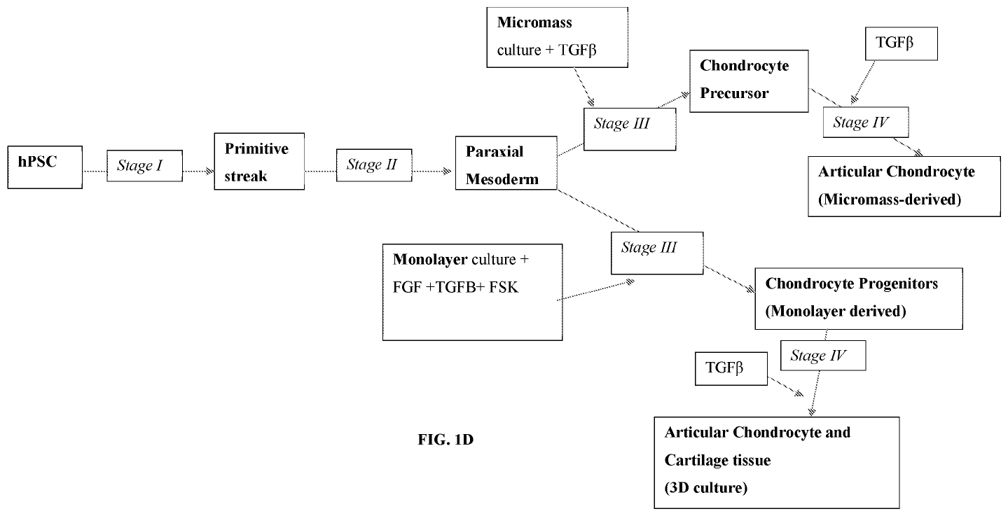

Two sequential rounds of re-plating were performed without any expansion in monolayer (FIGS. 1A-1C). The resulting tissues were cartilaginous, although they can be reduced slightly in size with each passage. They respond appropriately to TGFβ (for articular cartilage) and BMP (growth plate cartilage) throughout all passages. n=3 (or more) for each time point.

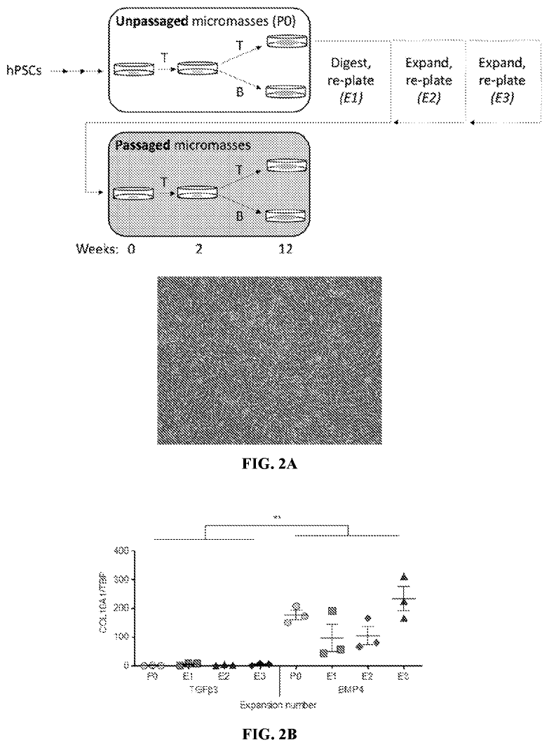

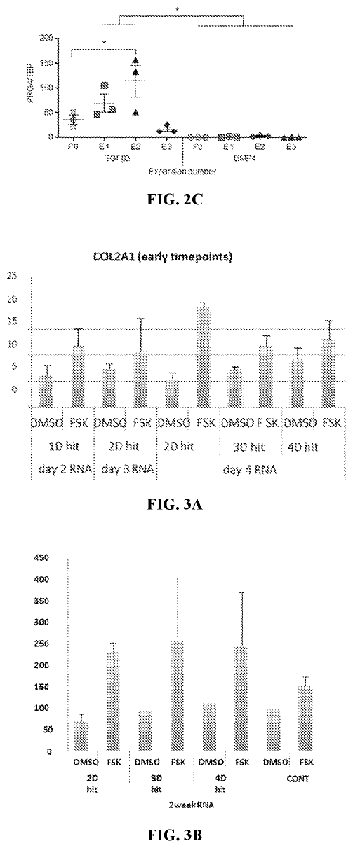

12-week-old TGFβ-treated chondrocytes in micromass (P0) were dissociated and cells were directly replated into new micromasses (E1) or expanded once in monolayer before re-plating them in micromass (E2), or expanded twice in monolayer before re-plating them in micromass (E3) (FIGS. 2A-2C). The histology confirms that these cells retain cartilage potential, and they respond to the two treatments (TGFB for articular cartilage and BMP4 for growth plate cartilage). The qPCR results suggest that COL10A1 comes back on with each passage following BMP4 treatment (as expected). PRG4 levels rebound nicely as well after passaging. A total of n=3-6 tissues were analyzed per timepoint. Expansion media contained 2% serum.

Phenotype of 12-week-old micromass-derived chondrocytes as they are expanded in monolayer (FIG. 2A). The cells were expanded in monolayer as described. It resulted in about 2-3-fold increase in yield. Cells expanded in monolayer acquire a mesenchymal stem cell-like phenotype, and chondrogenic phenotypes are restored upon high density micromass plating or encapsulation.

### Example 2: Expansion/Replating of 6-Week-Old Micromass-Derived Chondrocytes

### Materials and Methods

Chondrocytes within articular cartilage tissues (6-week-old micromass tissues) were dissociated from their matrix and expanded in monolayer prior to replating in micromass culture or biomaterial encapsulation for cartilage tissue formation. For monolayer expansion, approximately 500,000 to 2 million cells were plated per well on 6 well dishes coated with gelatin, collagen, or fibronectin, in culture condition of DMEM (plus ITS/proline/Dex)+2% FBS+Ascorbic acid or serum free culture media supplemented with TGFβ and FGF, or in serum-free media with TGFβ, FGF, and FSK. Expanded cells were cultured until confluent (˜3-5 days), and then trypsinized and replated in additional monolayer expansion or plated into micromass or encapsulated in biomaterials (data not shown).

### Results

Micromass-derived chondrocytes (6 weeks old) were expanded in 2% serum media and retained ability to make cartilage in new micromass or when encapsulated in biomaterial after passaging.

Micromass-derived chondrocytes were expanded and passaged in 2% serum media, in serum-free media with TGFβ and FGF, or in serum-free media with TGFβ, FGF, and FSK, for up to 3 passages and retained ability to make cartilage tissue in new micromass or when encapsulated in biomaterial after passaging.

These studies show that:
1. Micromass-derived chondrocytes can be expanded and retain cartilage forming potential (e.g., after 6 weeks and after 12 weeks). This is confirmed following expansion in both serum-containing and serum-free media
2. Micromass-derived chondrocytes can be serially passaged in micromass for 12 weeks each (up to three passages) and retain cartilage forming potential.

### Example 3: Inducing Chondrocytes in Monolayer Culture

### Materials and Methods

100,000-200,000 paraxial mesoderm cells were plated per well on 24-well plates with culture condition of serum-free differentiation media supplemented with FGF and/or TGFB and/or FSK or no supplements. Cells are cultured for 1, 2, 3, or 4 and up to 6 weeks in media.

### Results

Monolayer is initiated by seeding cells at low density in a tissue culture vessel. This differs from micromass, which is initially seeded at very high density in a ‘spot’. Monolayer cultures require several days or weeks to become confluent and chondrogenic induction can occur. It was observed that areas that become more confluent or dense underwent chondrogenesis first (this is a known phenomenon in cartilage cell culture). Base media for monolayer differs in composition of nutrients and supplements, in addition to differences in growth factors or molecules.

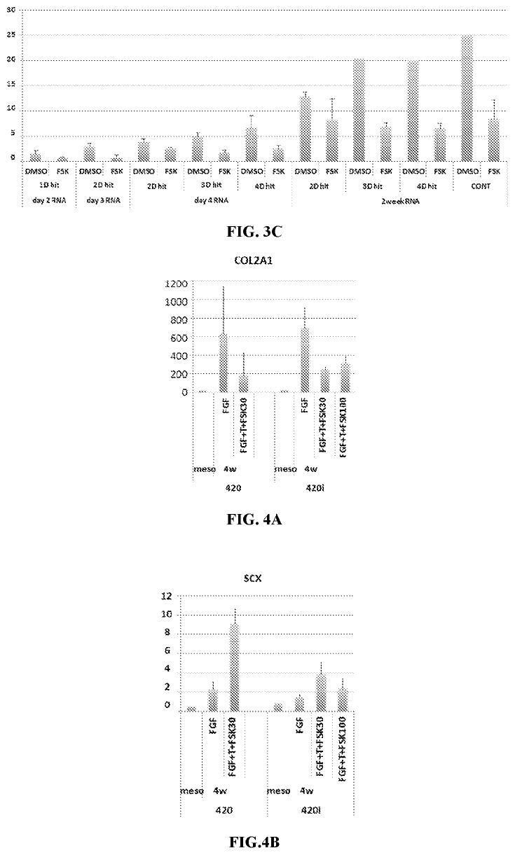

Forskolin (FSK) improved cartilage output in monolayer acutely (1 day) and longer term (2 weeks). Gene expression of COL2A1 in monolayer after 2, 3, 4 days or 2 weeks in presence of TGFB and FGF is shown in FIGS. 3A and 3B. SCX expression is reduced with FSK in presence of TGFB and FGF. SCX expression in monolayer after 2, 3, 4 days or 2 weeks is shown in FIG. 3C.

### Example 4: Phenotype and Gene Expression Patterns of Monolayer-Derived

### Chondrocytes Derived in Different Compositions

### Materials and Methods

Two starting populations (day 14 paraxial mesoderm/meso):

420=4 μM BMP inhibitor Dorsomophin and 20 ng/ml FGF in stage 2; 420 mesoderm shows tendencies towards tendon and less chondrogenic potential (in general in most experiments). 420i mesoderm=420+I=treated with a Wnt inhibitor IWP2 from day 3-5 of differentiation/stage 2, has increased chondrogenic potential. Five experiments were conducted, and qPCR tests were performed on the cells after 4 weeks and the data was pooled and analyzed.

In a second test, 420 and 420i paraxial mesoderm cells were plated in micromass cultures supplemented with TGFβ and qPCR was performed after 12 weeks of micromass expansion.

In a third test, 420 and 420i paraxial mesoderm cells were plated in serum-free monolayer culture with no additional factors and qPCR was performed following 4 weeks of culture. A second group of 420 and 420i paraxial mesoderm cells were plated in serum-free monolayer culture supplemented with either FGF alone or with a mixture of FGF, TGFβ and Forskolin (FSK). qPCR was performed after 4 weeks in culture. Each condition had a sample size of 3.

### Results

Chondrogenic monolayer phenotypes at 4 weeks old are all chondrogenic with 420i mesoderm starting population.

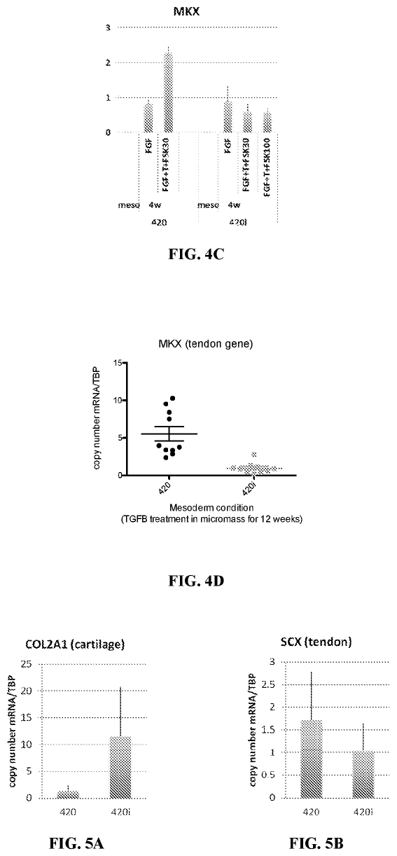

Gene expression including COL2A1 (cartilage gene), SCX (tendon gene), and MKX (tendon gene) of chondrocytes derived in monolayer after 4 weeks in different media compositions was analyzed (FIG. 4A-4C). qPCR data show that 420i has higher cartilage gene expression and 420 alone has higher tendon/ligament gene expression (SCX and Mohawk, or MKX expression). 420 alone has higher tendon/ligament gene expression in 12-week-old TGFβ-treated micromasses (Mohawk, or MKX expression) (FIG. 4D). The 420i-induced mesoderm also demonstrated higher chondrogenic potential in monolayer cultures after 4 weeks, shown by higher COL2A1 expression (a cartilage associated gene) in 420i cultures and higher SCX expression (a tendon gene) in 420 cultures when cultured in media that was not supplemented with any additional factors (FIGS. 5A and 5B). In media supplemented with FGF, 420-induced mesoderm demonstrated higher chondrogenic potential in monolayer cultures after 4 weeks shown by higher COL2A1 expression (a cartilage associated gene) in 420i cultures and higher SCX expression (a tendon gene) in 420 cultures (FIGS. 5C and 5D).

These results demonstrate that paraxial mesoderm produced in monolayer culture with different compositions of media may have different cartilage potential for therapeutic purposes.

### Example 5: Role of Forskolin in Inducing Chondrogenesis in Absence of FGF or TGFB

### Materials and Methods

100,000-200,000 paraxial mesoderm cells were plated per well on 24-well plates with culture condition of serum-free differentiation media supplemented with or without FSK. Cells were cultured for 2, 3, or 4 weeks in media.

### Results

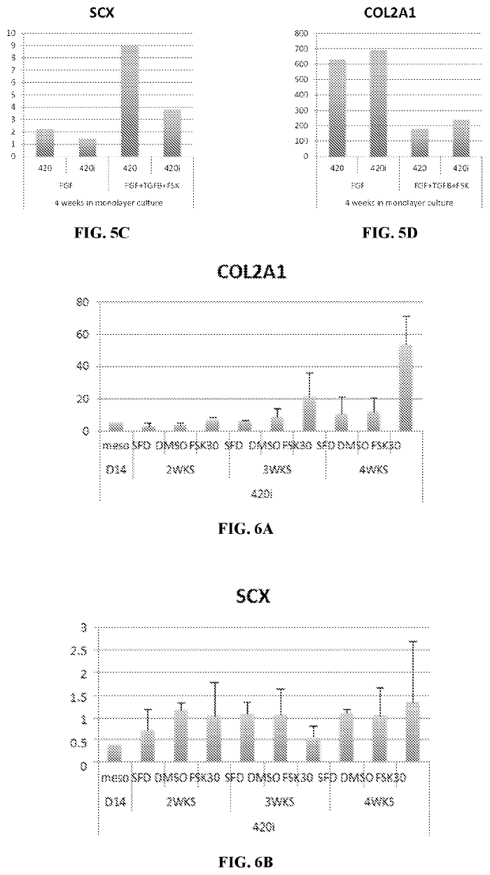

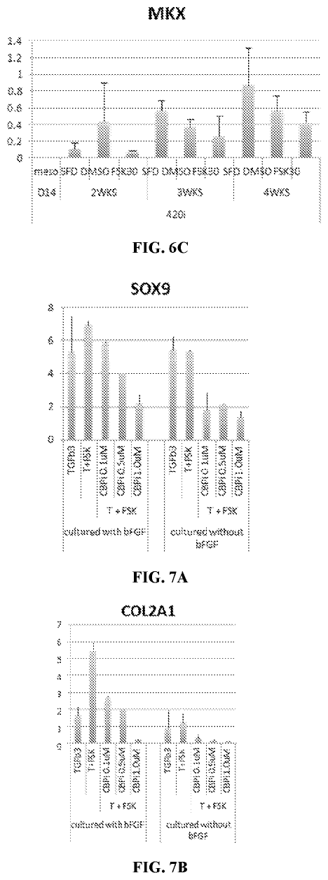

In the absence of exogenous TGFB/BMP agonists, endogenous TGFβ/BMP signaling must be present (usually at regions where cells are more confluent/higher density) for chondrogenesis to occur. In monolayers cultured without TGFβ and FGF, FSK (alone) induced cartilage. DMSO is vehicle control for FSK (reconstituted in DMSO). Increased chondrogenic gene expression (COL2A1) with FSK, minimal change or decrease in tendon genes (SCX and MKX) (FIGS. 6A-6C).

### Example 6: Effects of FSK are Dampened by CBP Inhibitor

### Materials and Methods

100,000-200,000 paraxial mesoderm cells were plated per well on 24-well plates with culture condition of serum-free differentiation media supplemented with TGFβ, with or without FGF and/or FSK and/or a CREB-binding protein inhibitor (CBPi). Cells were cultured for 1, 2, 3, or 4 weeks in media.

### Results

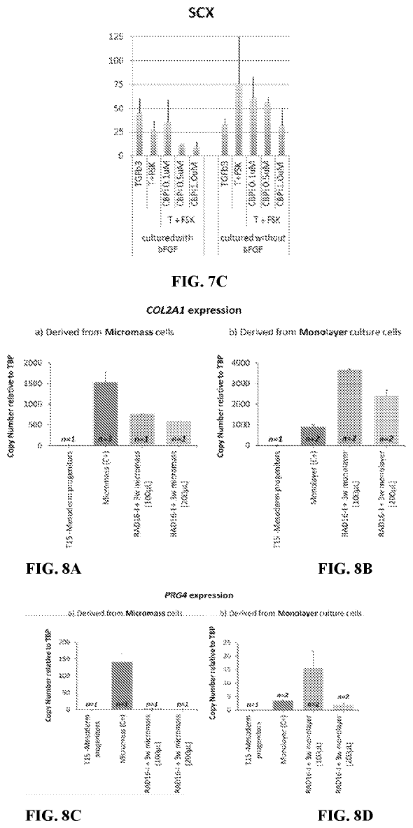

Effects of increased cartilage and/or tendon observed with FSK were dampened by CBP inhibitor, suggesting FSK's effect is mediated via PKA/CREB (FIGS. 7A-7C).

### Example 7: hPSC-Derived Chondrocytes can be Cryopreserved and Retain Cartilage Forming Potential Upon Thawing

### Materials and Methods

Micromass tissues (aged 6 weeks old) were dissociated by collagenase treatment and were resuspended in cryopreservation media: 50% FBS+10% DMSO+40% IMDM. Approximately 5 million cells aliquoted into each vial (testing a range from 2-10 million cells per vial) and immediately stored in −80° C. for 2 days followed by transfer to liquid nitrogen.

Frozen cells were thawed and replated in micromass or encapsulation in biomaterial (RAD16-I/Puramatrix) and analyzed for viability and cartilage forming potential after 6 weeks.

### Results

Micromass derived chondrocytes are viable after cryopreservation/thaw. Viability/Yield of micromass-derived chondrocytes after thawing is summarized in Table 1 below.

TABLE 1
Viability and yield of micromass-
derived chondrocytes after thawing
5 Million cells per each Storage Vials

| | Thaw 1 | Thaw 2 |
|---|---|---|
| Total Cells | 4820000 | 4340000 |
| Live Cells | 4400000 | 4120000 |
| Viability | 91% | 95% |
| Recover rate | 96% | 87% |
| Cumulative Yield | 88.00% | 82.40% |

After a cycle of freeze-thaw, micromass-derived chondrocytes generated a cartilage tissue. The histology confirms that cartilage following thawing cycles, appears to be of better quality (i.e., as observed by more uniform Toluidine blue staining, thicker tissue) compared the cartilage generated from original micromass prior to cryopreservation. Thus, cryopreserved micromass-derived chondrocytes retain their cartilage potential and their ability to make cartilage tissue post-thaw.

Monolayer-derived chondrocytes could also be cryopreserved and/or passaged and they retained cartilage-forming potential similar to micromass-derived chondrocytes.

### Example 8: Monolayer-Derived Chondrocytes Encapsulated in Biomaterial for Tissue

### Engineering of Implants

Materials and Methods Chondrocytes from monolayer cultures were dissociated from their original matrix, or thawed from cryopreservation state, and encapsulated in RAD16-I/PURAMATRIX™ scaffolds using Millicell-CM inserts for 6 weeks. Each insert was filled with 12,000 cells/μL cell/RAD16-I solution and polymerized through pH equilibration. Tissue engineered constructs were cultured in serum-free media containing ascorbic acid, dexamethasone, proline, insulin, and transferrin cell culture media supplement (ITS) and TGFβ for 6 weeks.

### Results

Cartilage formation from hESC-derived chondrocytes derived from monolayer cultures in the PURAMATRIX™ system were compared to micromass-derived chondrocytes using histological analysis. These constructs were cultured before toluidine blue staining analysis. Toluidine blue staining indicated proteoglycan rich matrix (ideal for cartilage) and tissues were uniform in thickness, ideal for implantation. TGFβ+FGF+FSK-induced monolayer-derived chondrocytes were successfully encapsulated for tissue engineering, and they are comparable to micromass-induced chondrocytes.

Monolayer-derived chondrocytes behaved similarly to micromass-derived chondrocytes in terms of their expression of COL2A1 and PRG4 when encapsulated in biomaterial for tissue engineering of implants for 6-8 weeks (FIGS. 8A-8D).

More nuanced cartilage potential of monolayer-derived cells was observed. Differentially induced monolayer-derived chondrocytes are likely to produce distinct cartilage tissues upon seeding into micromass culture.

Micromasses were seeded with monolayer-derived chondrocytes and were treated with TGFβ to induce articular cartilage (i.e., to reproduce “superficial zone-like” chondrocytes that express PRG4) or with BMP to induce growth plate cartilage (GPC; i.e., to produce hypertrophic chondrocytes that express COL10A1). Monolayer-derived chondrocytes were generated with the following compositions: FGF alone or TGF+FGF+FSK30.

Plating monolayer-derived chondrocytes in TGFB-treated micromass induces articular-like cartilage phenotype. Plating monolayer-derived chondrocytes in BMP4-treated micromass culture induces growth plate cartilage phenotype. Hypertrophic chondrocyte gene or GPC gene COL10A1 is higher in BMP micromasses (FIG. 9A), regardless of monolayer treatment prior to micromass culture. Articular cartilage gene (PRG4) is higher in TGFB micromasses. (FIG. 9B), as expected, and it is higher in micromasses derived from FGF+TGFβ+FSK treated cells. Chondrocyte progenitors derived in monolayer with either FGF alone or TGFB+FGF+FSK can generate articular and growth plate cartilage later in micromass (FIGS. 9A-9F)

In summary, monolayer-derived chondrocytes can be influenced later to become articular or growth plate cartilage. Adding an exogenous TGFβ agonist was not required for FGF-treated cells to become chondrocytes.

### Example 9: Derivation and Molecular Characterization of Human Pluripotent Stem Cells (hESCs/iPSCs) Capable of Giving Rise to Both Growth Plate and Articular Cartilage

Using a modified directed differentiation approach based on previously described methods (Craft et al., Nat Biotechnol., 2015, 33(6):638-645; PMID: 25961409), derived chondrocytes from human pluripotent stem cells (i.e., hESCs/iPSCs) capable of giving rise to both growth plate and articular cartilage were generated. The objectives of this study were two-fold. The objective was to determine the molecular mechanisms governing chondrocyte cell fate and differentiation by comprehensively probing the molecular signatures of the generated hESC-derived chondrocytes, along with chondrocytes derived from developing mouse and human cartilage. The transcriptomes of hESC-derived articular chondrocytes (AC) and growth plate chondrocytes (GPC) by bulk RNA-sequencing (RNA-seq) were characterized. The findings indicated (1) lineage-specific gene expression whose directionality overlapped significantly with the gene expression differences observed between epiphyseal and growth plate cartilage during human development, (2) accompanying regulatory landscapes of hESC-derived chondrogenic lineages, as well as mouse chondrocytes isolated by cell sorting, via refined ATAC-sequencing (ATAC-seq) protocols (i.e., protocols as previously described in Gui et al., Elife, 2017, 5(6) e29329, PMID: 29205154; Richard et al., 2020, Cell, 181(2): 362-381, PMID: 32220312), (3) identification of putative gene regulatory networks specific to either growth plate or articular chondrocytes via integration of the collective transcriptomic and epigenetic datasets, and (4) evidence supporting representative regulatory networks for RUNX2 in growth plate chondrocytes and for RELA (aka Transcription factor p65) in articular chondrocytes.

### Results

Trancriptomic Profiles of hESC-Derived and Human Fetal Chondrocytes

Previous studies by the inventor have shown that hESC-derived chondrogenic cells/tissues acquire characteristics reminiscent of developing human articular or growth plate chondrocytes/cartilage following treatment with TGFβ 3 (TGFβ) or BMP4 (BMP), respectively (PMID: 25961409). Both cartilage tissues are similar in size, and rich in proteoglycans as confirmed by histological assessments. The surface layer of the hESC-derived articular cartilage is smooth and contains flattened chondrocytes arranged parallel to the surface, while its deeper zone of this tissue contains chondrocytes that are relatively uniform in size and are evenly distributed within their extracellular matrix. Chondrocytes within the BMP-treated cartilage matrix are relatively larger, or hypertrophic.

In previous studies, a candidate based (qRT-PCR and immunohistochemistry) approach was taken to confirm that lineage-specific genes were specifically expressed in each respective tissue (Craft et al., Nat Biotechnol., 2015, 33(6):638-645; PMID: 25961409), such as PRG4 expression in the articular lineage and COL10A1 in the growth plate lineage. Beyond these classic chondrocyte markers, and information gained from studies in other species, understanding of the transcriptional profiles corresponding to human articular and growth plate cartilage remains limited. To generate unbiased transcriptomic profiles for each of these lineages, a series of RNA-sequencing experiments on both chondrogenic lineages derived in parallel from 7 independent differentiations of the H9 hESC line were performed. Twelve-week-old chondrocytes/cartilage tissues from 4 experiments (n=6 tissues in total) were collected to facilitate robust comparisons between the lineages. RNA-sequencing was also performed on chondrocytes isolated from developing human epiphyseal or growth plate cartilage to serve as an in vivo reference for each cell type. Primary chondrocytes were isolated from the left or right distal femur from an embryonic day (E)67 fetal donor sample. Genes that were differentially expressed (DEGs) between respective cartilage lineages, in vitro or in vivo, are studied.

The resulting transcriptomic signatures of each chondrogenic lineage were distinct, as indicated by Principal Component Analysis (FIG. 10A). Principle component 1 (PC1) represents differences observed between the in vitro and in vivo samples (FIG. 10A, squares vs. circles/triangles). Within each experimental group, the articular and growth plate samples were separated along PC2 (FIG. 10A, PC2). As we predicted from candidate gene expression trends, the hESC-derived articular chondrocytes (orange circles/triangles) clustered closest to the fetal epiphyseal cartilage (red squares), while the hESC-derived growth plate cartilage clustered (light blue circles/triangles) closest to the fetal growth plate cartilage (dark blue squares). Differences between articular and growth plate cartilage were more pronounced for hESC-derived chondrocytes than for their in vivo counterparts, as indicated by the greater distance in separation along the PC2 axis.

The top 40 genes with the highest degree of differential expression between hESC-derived articular and growth plate chondrocytes are UCMA, R3HDML, CHI3L1, GPR171, PRG4, LYPD1, COL15A1, TNMD, COMP, MEOX1, ADORA1, FAP, FGF1, DOC2B, CD79B, MMP3, GALNT16, GH1, PAX1, AIF1, IL17B, MC3R, TRIM53BP, CD24P4, SCNNIB, SP7, VNIR40P, SCLBA3, LRRC38, APOB, COLOAJ, TRIM49D1, ALPL, CXCL3, PRB2, TRIM53AP, MEPE, HDC, PANX3, and IBSP (heat map not shown). Most of these genes exhibited similar differential expression in fetal epiphyseal and fetal growth plate chondrocytes (data not shown). This trend persisted and achieved statistical significance (p-value <0.05) when the dataset was expanded to the top 200 differentially expressed genes (FIG. 10B, compare top and bottom graphs). Similar results were obtained when starting with the top differentially expressed genes between fetal epiphyseal and fetal growth plate chondrocytes (data not shown). Finally, gene-set enrichment analyses (GSEA) were performed on the set of genes upregulated in hESC-derived articular or hESC-derived growth plate cartilage. For hESC-derived articular cartilage enrichment was found for terms relating to (1) ECM organization, (2) response to TGF stimulus, and (3) collagen processes (Table 2). For hESC-derived growth plate cartilage, enrichment was found for terms relating to (1) ossification, (2) ECM organization and (3) cartilage development (Table 3). Similar enrichment terms were obtained when the same analysis was performed on genes upregulated in fetal epiphyseal cartilage or fetal growth plate cartilage. Taken together, these data lend strong support to the notion that the two chondrogenic cell types derived from hESCs represent bona fide chemically induced articular and growth plate chondrocytes.

TABLE 2
Gene-set enrichment analyses for upregulated genes in hESC-derived articular cartilage
HESC-derived articular cartilage

| ID | Description | GeneRatio | BgRatio | pvalue |
|---|---|---|---|---|
| GO:0030198 | extracellular matrix organization | 12/88 | 361/16722 | 4.0158E−07 |
| GO:0043062 | extracellular structure organization | 12/88 | 362/16722 | 4.1359E−07 |
| GO:1902042 | negative regulation of extrinsic apoptotic | 4/88 | 37/16722 | 4.1414E−05 |
| | signaling pathway via death domain | | | |
| | receptors | | | |
| GO:0090197 | positive regulation of chemokine secretion | 3/88 | 15/16722 | 6.1212E−05 |
| GO:0090196 | regulation of chemokine secretion | 3/88 | 16/16722 | 7.5052E−05 |
| GO:0008625 | extrinsic apoptotic signaling pathway via | 5/88 | 84/16722 | 8.0117E−05 |
| | death domain receptors | | | |
| GO:0090195 | chemokine secretion | 3/88 | 18/16722 | 0.00010853 |
| GO:1900047 | negative regulation of hemostasis | 4/88 | 53/16722 | 0.00017221 |
| GO:0032722 | positive regulation of chemokine | 4/88 | 56/16722 | 0.00021343 |
| | production | | | |
| GO:0001525 | angiogenesis | 10/88 | 482/16722 | 0.00021769 |
| GO:0071560 | cellular response to transforming growth | 7/88 | 232/16722 | 0.00022111 |
| | factor beta stimulus | | | |
| GO:0071559 | response to transforming growth factor | 7/88 | 238/16722 | 0.00025836 |
| | beta | | | |
| GO:0050730 | regulation of peptidyl-tyrosine | 7/88 | 244/16722 | 0.00030051 |
| | phosphorylation | | | |
| GO:0009595 | detection of biotic stimulus | 3/88 | 29/16722 | 0.00046608 |
| GO:0021696 | cerebellar cortex morphogenesis | 3/88 | 29/16722 | 0.00046608 |
| GO:0090287 | regulation of cellular response to growth | 7/88 | 263/16722 | 0.00047174 |
| | factor stimulus | | | |
| GO:0002685 | regulation of leukocyte migration | 6/88 | 189/16722 | 0.00048412 |

TABLE 3
Gene-set enrichment analyses for upregulated genes in hESC-derived growth plate cartilage
HESC-derived growth plate

| ID | Description | GeneRatio | BgRatio | pvalue |
|---|---|---|---|---|
| GO:0001958 | endochondral ossification | 6/166 | 28/16722 | 2.7483E−07 |
| GO:0036075 | replacement ossification | 6/166 | 28/16722 | 2.7483E−07 |
| GO:0031214 | biomineral tissue development | 10/166 | 158/16722 | 4.0086E−06 |
| GO:0110148 | biomineralization | 10/166 | 158/16722 | 4.0086E−06 |
| GO:0090287 | regulation of cellular response to growth | 11/166 | 263/16722 | 6.4961E−05 |
| | factor stimulus | | | |
| GO:0071695 | anatomical structure maturation | 10/166 | 230/16722 | 0.000102 |
| GO:0002062 | chondrocyte differentiation | 7/166 | 121/16722 | 0.00020474 |
| GO:0051216 | cartilage development | 9/166 | 205/16722 | 0.00021151 |
| GO:0001649 | osteoblast differentiation | 9/166 | 207/16722 | 0.00022737 |

In addition to the identification of differentially expressed genes in hESC-derived articular and growth plate chondrocytes after 12 weeks of differentiation, we also identified differentially expressed genes between these two lineages at earlier timepoints in a set of new experiments, namely 4 weeks and 8 weeks of differentiation, and we included corresponding cultures after 12 weeks. For hESC-derived articular cartilage at 4 weeks, enrichment was found for terms relating to (1) cartilage development, (2) skeletal system development and (3) extracellular matrix, among others (data not shown).

For hESC-derived growth plate cartilage at 4 weeks, enrichment was found for terms relating to (1) chondrocyte differentiation, (2) extracellular matrix organization and (3) collagen fibril organization, among others (data not shown).

For hESC-derived articular cartilage at 8 weeks, enrichment was found for terms relating to (1) extracellular matrix, (2) response to transforming growth factor and (3) collagen fibril organization, among others (data not shown).

For hESC-derived growth plate cartilage at 8 weeks, enrichment was found for terms relating to (1) ossification, (2) extracellular matrix organization and (3) bone mineralization, among others (data not shown).

In replicate hESC-derived articular cartilage at 12 weeks, enrichment was found for terms relating to (1) extracellular matrix organization, (2) collagen fibril organization and (3) skeletal system development (Table 2 and 3), among others.

In replicate hESC-derived growth plate cartilage at 12 weeks, enrichment was found for terms relating to (1) endochondral ossification, (2) endochondral bone morphogenesis and (3) skeletal system morphogenesis (Table 2 and 3), among others.

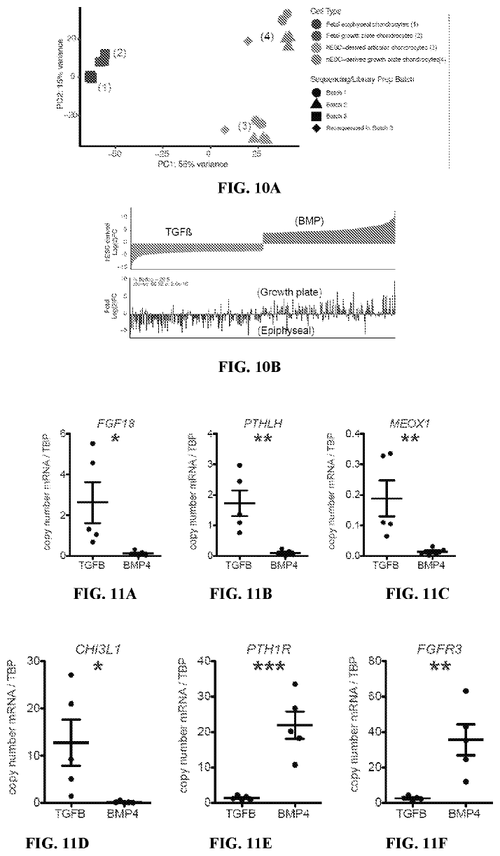

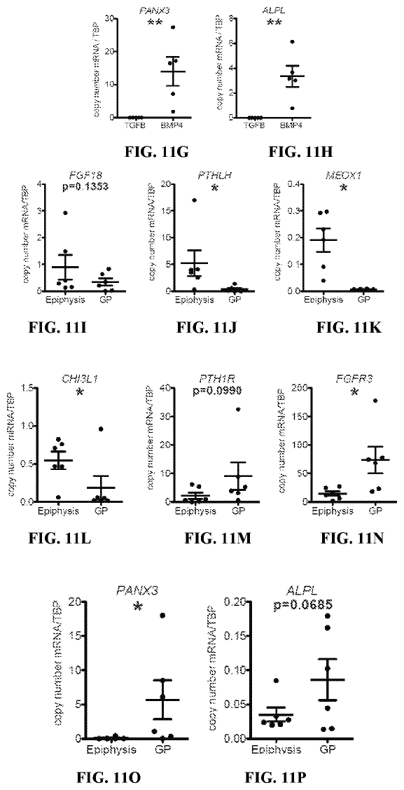

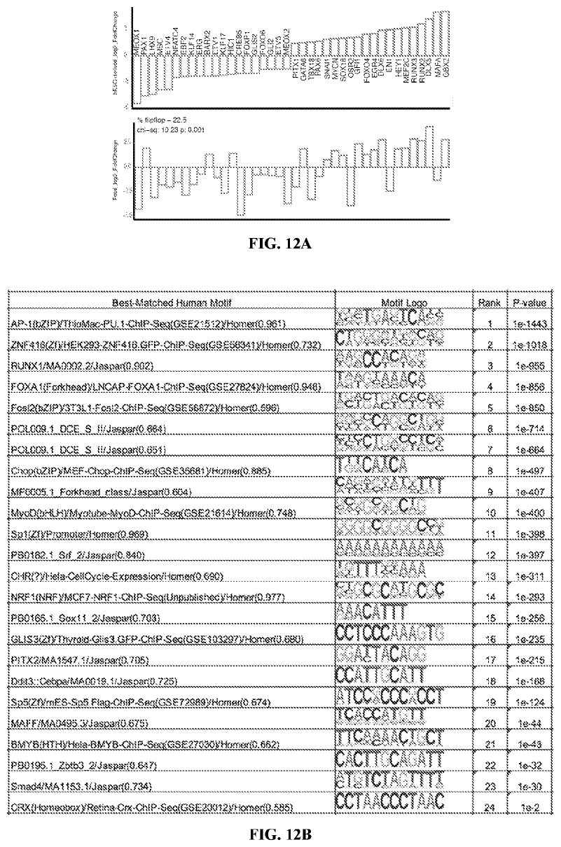

A subset of the Differentially Expressed Genes (DEGs) identified by RNA-seq was validated using in situ hybridization (ISH), quantitative RT-PCR (qPCR), and immunohistochemistry (IHC) on additional cartilage tissues derived from hESCs, epiphyseal and growth plate chondrocytes from the distal femur and proximal tibia of fetal donor specimens at stages (Embryonic) E59-E72, when tissues are actively differentiating, and developing fetal joints/growth plates (FIGS. 11A-11P.). Some of the top DEGs, such as PRG4 and COMP, have been previously demonstrated in cartilage biology, while the observed expression of other DEGs extracted from the current dataset in cartilage, such as COL15A1 and EFHDa1, were previously unknown in cartilage biology.

Spatial resolution of RNA or protein expression was used to identify DEG expression in specific zones of cartilage. To localize mRNA expression in situ, RNAscope was performed on hESC-derived cartilage tissues as well as the fetal knee joint and distal femur growth plate sections using probes recognizing COL2A1, PRG4, TNMD, and COL10A1. Type II collagen, encoded by the gene COL2A1, is a major structural component of both articular and growth plate cartilage, and as such, expression is observed in the cartilaginous structures both in vitro and in vivo (micrograph not shown). PRG4 is expressed in the superficial layer of the hESC-derived TGFβ-treated articular cartilage and absent in the BMP4-treated growth plate-like cartilage (micrograph not shown). Similarly, in vivo, PRG4 is expressed in the superficial zone of fetal articular cartilage, as well as the intra-articular ligaments and meniscus, and is absent in the growth plate. COL10A1 mRNA is detected in the hESC-derived BMP4-treated growth plate cartilage, but not in the TGFβ-treated articular cartilage, consistent with expression patterns found in the fetal knee, where COL10A1 is expressed in the hypertrophic chondrocytes of the growth plate but not in the epiphyseal chondrocytes (micrograph not shown). Tenomodulin (TNMD), a well-known marker of tendon fate (PMID: 15632070), was a top DEG in the articular/epiphyseal lineages. Of interest, TNMD expression was detected in the most superficial layers of the hESC-derived articular cartilage and the fetal knee epiphyseal cartilage. TNMD was also expressed in the intra-articular ligaments in vivo, as expected.

Several DEGs were further validated on hESC-derived chondrocytes (FIGS. 11A-11H) and fetal chondrocytes from the distal femur and proximal tibia of three developmental timepoints (E59 (Carnegie Stage 23), E67 and E72; FIGS. 11I-11P) using qPCR. Receptor-ligand pairs, FGFR3 and FGF18, and PTH1R and PTHLH are known to be differentially expressed between articular (FGF18, PTHLH) and growth plate cartilages (FGFR3, PTH1R) (PMID: 31290205, PMID: 12960068, PMID: 23060229, 15781473, PMID: 27142453, PMID: 8314082). As predicted by previous studies, expression levels of FGF18 and PTHLH were significantly higher in hESC-derived articular chondrocytes, and levels of FGFR3 and PTH1R were significantly higher in the hESC-derived growth plate chondrocytes. Similar patterns were observed in fetal chondrocytes. Pannexin 3 (PANX3), the second highest DEG in the growth plate lineage, was previously found to promote chondrogenic differentiation in prehypertrophic and hypertrophic chondrocytes of the growth plate (PMID: 20404334), and we found its expression is significantly higher in both hESC-derived and fetal growth plate chondrocytes. Alkaline phosphatase (ALPL), known to be expressed in both hypertrophic chondrocytes and osteoblasts, plays a role in the mineralization of bone (PMID: 11850436). As expected, ALPL was also more highly expressed in hESC-derived and fetal growth plate chondrocytes compared to their respective articular/epiphyseal cartilage. In the articular lineage, chitinase-3 like protein 1 (CHI3L1, also known as YKL-40) and mesenchyme homeobox 1 (MEOX1) were found to be top DEGs, and their lineage-specific expression were confirmed in vitro and in vivo. CHI3L1 expression has been previously described in cultured chondrocytes and osteoarthritic cartilage (PMID: 11315922, PMID: 15015934), while MEOX1 is most well-known for its role in somitogenesis and axial skeleton formation (PMID:19520072).

Using IHC, DEG-encoded proteins were detected in hESC-derived cartilages, as well as developing phalangeal (E70) and knee joints (E59). Cartilage Oligomeric Matrix Protein (COMP), a non-collagenous extracellular matrix protein expressed in cartilage, ligaments, and tendon (PMID: 16340129, PMID: 24558358, PMID: 16542502), was more highly expressed in hESC-derived articular cartilage, but was not differentially expressed between the fetal epiphysis and fetal growth plate, consistent with the overlap in similar chondrocyte cells between these two samples (fetal cartilaginous elements are continuous structures and the dissection location was approximate). COMP was detected both in the matrix and in cells within the hESC-derived articular cartilage tissue, but found only intracellularly in hESC-derived growth plate tissue. In both the metacarpophalangeal and knee joints, COMP is detected in the matrix of both the epiphyseal and growth plate cartilage, consistent with the fetal RNA-seq data. It is also detected in ligaments, and perichondrium tissues. SP7 (also known as Osterix), a transcription factor expressed essential for growth plate chondrocyte and osteoblast differentiation (PMID: 11792318) (PMID: 21075078).), was significantly higher in hESC-derived and fetal growth plate cartilages. At the protein level, it is localized to the nucleus of hypertrophic chondrocytes within the hESC-derived growth plate cartilage and the fetal growth plate, but was not detectable in the epiphyseal chondrocytes at the joint surface.

The chosen unbiased transcriptomic approach also uncovered potential previously undescribed markers of articular and growth plate chondrocytes. Type XV collagen (COL15A1) is a non-fibrillar basement membrane-associated collagen protein that has been previously detected in the perichondrium around bones and in mesenchymal stem cells undergoing osteogenic differentiation (PMID: 11827796, PMID: 19365806). While not differentially expressed in the fetal tissues, COL15A1 was significantly higher in hESC-derived articular cartilage compared to hESC-derived growth plate cartilage. We localized the highest level of type XV collagen in the matrix of the superficial zone of hESC-derived articular cartilage, and it was also detected within cells of the deeper zone of the articular cartilage and some cells within the growth plate cartilage. Type XV collagen is expressed in the matrix of the epiphysis of the metacarpophalangeal joint, and at the surface of the knee joint cartilages, but is absent in the matrix surrounding hypertrophic chondrocytes of the growth plates. These data suggest COL15A1 expression may be specific to the superficial zone of articular cartilage. EF-hand domain-containing protein 1 (EFHD1) expression was significantly higher in both hESC-derived and fetal growth plate chondrocytes. EFHD1 is a calcium-binding protein localized to the inner mitochondrial membrane, previously undescribed in cartilage (PMID: 33537316). EFHD1 protein was localized to the cytoplasm of BMP4-treated hypertrophic chondrocytes, and hypertrophic chondrocytes in the fetal growth plates, but not in articular or epiphyseal cartilage, as expected. These data indicate EFHD1 is specifically expressed in hypertrophic chondrocytes of the growth plate.

Collectively, these experiments underscored the utility of the established in vitro differentiation methods to define chondrocyte lineages, and further illustrated the strength of the described transcriptomic datasets in identifying previously described markers as well as undescribed markers and potential regulators of articular and growth plate cartilage.

Cataloging Gene Expression and Chromatin Accessibility Differences Between hESC-Derived Articular Chondrocytes and hESC-Derived Growth Plate Chondrocytes

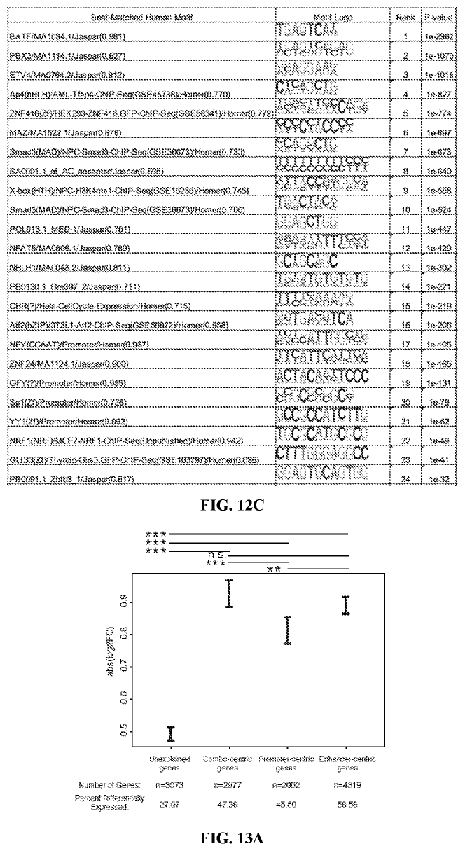

277 transcription factors (TFs) that were differentially expressed were identified in at least one of the four cell types profiled and for which a binding motif has been described. A subset of these (n=36) was more highly expressed in both hESC-derived and fetal articular chondrocytes, while a different subset (n=23) was more highly expressed in both hESC-derived and fetal growth plate chondrocytes. (Top 40 TFs shown in FIG. 12A). To refine this list of potential chondrogenic lineage regulators, their interactions with the genome in growth plate or articular chondrocytes were investigated by performing ATAC-seq (Ludwig et al., Cell Reports, 2019, 27, 3228-3240, PMID: 31189107). ATAC-sequencing (ATAC-seq) is a method used to characterize chromatin accessibility on a genome-wide basis, on a subset of hESC-derived chondrocytes that were used for transcriptomic analysis, in order to facilitate robust comparisons between gene expression and chromatin accessibility. For an in vivo comparison, ATAC-seq data was generated from a general population of mouse embryonic chondrocytes expressing Col2a1 or hypertrophic chondrocytes expressing Col10a1. Col2a1+ or Col10a1+ chondrocytes were isolated from E15.5 transgenic mice harboring fluorescent reporters driven by Col2a1 or Col10a1 regulatory elements using cell sorting. The genome-wide overlap of peaks found in the two types of human and mouse chondrocytes is summarized in Table 4. Col2a1+ chondrocytes may contain chondrocytes that also express Col10a1, and it is expected that Col10a1-+chondrocytes also express Col2a1, but this population is more restricted in its hypertrophic lineage.

TABLE 4
Summary of ATAC-seq peaks from mouse and human chondrocytes.

| | | | Mouse embryonic | | |
|---|---|---|---|---|---|
| | | | chondrocytes | | |
| | | | All | Col2+ | Col10+ |
| | | | 30,950 | 28,972 | 12,906 |
| hESC- | All Peaks | 37,780 | 13,687 | | |
| derived | (T + B) | | | | |
| chondrocytes | TGFB | 31,137 | | 13,381 | 9,223 |
| | (ADI peaks) | | | | |
| | BMP | 29,821 | | 12,471 | 9,070 |
| | (All peaks) | | | | |
| | TGFB | 11,571 | | 3,971 | 2,385 |
| | (Unique) | | | | |
| | BMP | 12,154 | | 2,584 | 1,754 |
| | (Unique) | | | | |

Profiling the hESC-derived chondrocytes by ATAC-seq and calling significant reproducible open-chromatin regions (i.e., peaks) revealed a total of 37,780 unique peaks, corresponding to putative regulatory elements. These regions were categorized on the basis of differential accessibility in either growth plate or articular chondrocytes, identifying 12,154 regions more accessible in growth plate chondrocytes and 11,571 more accessible in articular chondrocytes. These differentially accessible regions (DARs) suggest cell-type specific regulatory activity and are the focus of subsequent analyses. Examples of DARs identified in growth plate and articular chondrocytes are shown for the IHH and FGF1loci, respectively. Using the GREAT region-based association tool [PMID: 20436461], terms significantly associated with DARs from growth plate chondrocytes were identified, including anomaly of the limb diaphyses and ECM organization. Likewise, terms associated with DARs from articular chondrocytes were also identified, including ECM organization, collagen metabolic process, and osteoarthritis. The top 20 DEGs for each lineage in this subset of hESC-derived tissues, the accessibility of their corresponding promoters, and their respective cis-regulatory score (CRS, see below) are listed as follows, and are merely representative of a larger dataset:

(1) For hESC-derived articular (TGFβ-correlated) chondrocytes, the genses with high expression and promoter accessibility scores ranging from 0 to 2 include SERTAD4-AS1, PENK, ADORA1, FGF18, MCUB, FGF1, SNTTB1, SSC5D, ADAMTSL2, CD70 CILP2, COMP, ANGPTL6, SERTAD4, FAP, COL22A1, GALNT16, COL15A1, LYPD1 and CHI3L1. Conversely, these genes also exhibit low expression and promoter accessibility for hESC-derived growth plate (BMP-correlated) chondrocytes.

(2) For hESC-derived growth plate (BMP-correlated) chondrocytes, the DARs with high expression and promoter accessibility scores ranging from 0 to 2 include IRF6, S100P, COL10A1, LPAR3, IHH, LG14, MGST1, FST, SLC13A5, ADGRD1, FAM177B, FXYD3, TSPAN18, WDR86, VSTM2L, HOXB6, SPINK5, TOX2, and C5AR2. Conversely, these DARs also exhibit low expression and promoter accessibility for hESC-derived articular (TGFβ-correlated) chondrocytes. 57 TF-encoding genes were differentially expressed primarily in hESC-derived chondrocytes, 134 TF-encoding genes were differentially expressed primarily in fetal chondrocytes, and 86 genes encoding TFs overlapped between the hESC-derived and fetal chondrocytes (subset shown in FIG. 12A).

To begin to understand differential transcriptional regulation mechanisms in these two lineages, de novo motif analysis was used to identify over-represented transcription factor (TF) motifs in DARs specific to either articular or growth plate chondrocytes (FIGS. 12B and 12C). A handful of these TFs were also differentially expressed in the corresponding cell type; however, the majority was not The same two sets of DARs were further examined for enrichment of motifs belonging to TFs differentially expressed in the corresponding cell types. This yielded a reduced set of TFs, several of which were also observed in the de novo motif anlaysis (FIGS. 12B and 12C). This approach confirmed that motif enrichment is not substantially correlated with sequence complexity. Analysis of sets of lineage-specific DARs of nearby genes exhibiting lineage-specific expression revealed similar enrichments for motif occurrences of several of these TFs in both region sets, despite conditioning on lineage-specific expression of these factors (data not shown). For example, the top 10 highly expressed genes encoding TFs specific to the BMP lineage are IRF6, MAFA, EGR3, HOXB6, FOXA2, OSR2, RUNX3, RUNX2, TBX20, andMEF2C. These TF-encoding DEGs are downregulated in the TGFβ cell lineage. However, enrichment of binding motifs of these TFs (including that of MEF2C) in BMP and TGFβ DARs revealed non-specificity of motifs in the BMP lineage only, with the exception of RUNX2 and RUNX3 whose motifs were significantly enriched in BMP DARS but were not significantly enriched in TGFB DARs. Conversely, the top 10 highly expressed genes encoding TFs specific to the TGFβ cell lineage are POUF2, MSC, NFATC2, HIC1, EGR2, NFATC4, EBF2, PKNOX2, SOX15, and ERG (heat map data not shown). These TF-encoding DEGs are downregulated in the BMP cell lineage. However, enrichment of TF binding motifs (include that of POU2F2) in DARS were not specific to just the TGFB lineage, thus also demonstrating lack of significant specificity of TFs to this lineage. This suggests that a simplistic model of gene expression, wherein upregulation of a given TF is associated with increased accessibility of elements to which it may bind, and subsequently increased expression of its putative targets, is not sufficient to explain the gene regulatory network information captured by the described ATAC-seq/RNA-seq strategy.

Defining Hypothetical Gene Regulatory Networks and their Divergence Across Cell Lineage Development

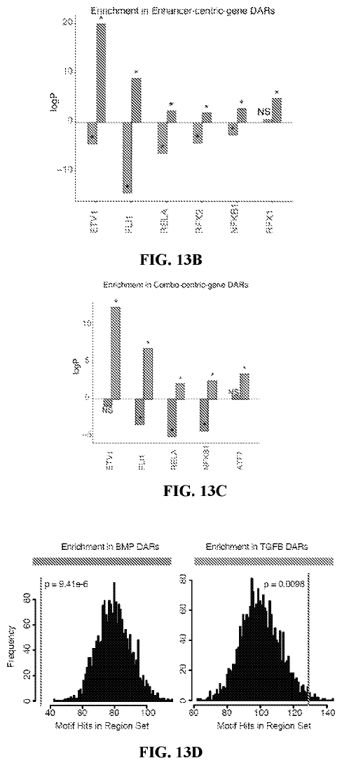

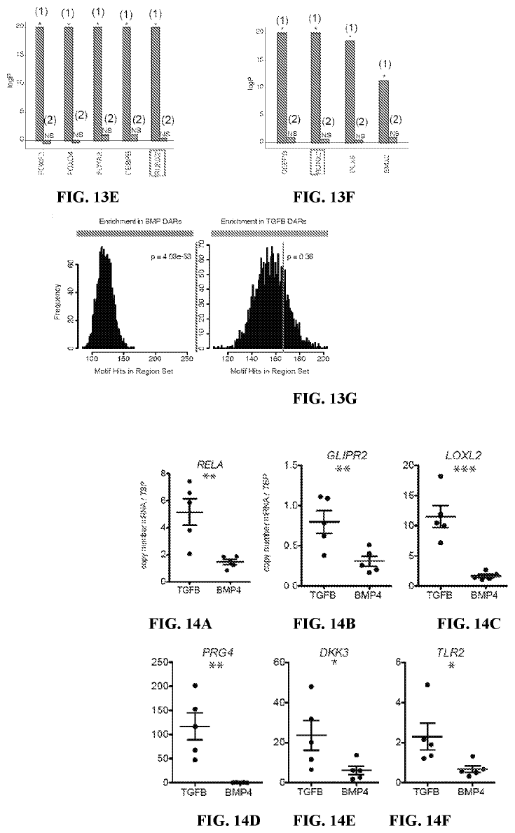

The ATAC-seq and RNA-seq datasets were integrated to better capture the regulatory behavior described in the sequencing datasets. The chosen approach defined three metrics of expression and accessibility at a given locus: 1) gene expression, 2) proximal (promoter) accessibility, and 3) distal (enhancer) activity, defined as a cis-regulatory score. For the top 20 DEGs, generally good correspondence was observed between these three metrics. However, expanding the scope of this integration approach to all DEGs made the correspondence less clear i.e. the genes exhibiting lineage-specific expression (as assessed by RNA-seq analysis) demonstrated varied and overlapping promoter accessibility (as assessed by ATAC-seq analysis). Potentially, multiple regulatory principles may be at play. By modifying an analytical approach described in previous studies describing the cis-regulatory behavior of immunological genes in mice (Yoshida et al., Cell, 2019, 176(4) 897-912, PMID: 30686579), the genes were classified into four different regulatory behaviors based on the proportion of variance in expression explained by chromatin accessibility within their respective loci. Briefly, these contain genes whose expression variance is best explained by: (1) variance does not clearly associate with chromatin accessibility (‘unexplained’, cluster 1); (2) a combination of promoter accessibility and distal cis-regulatory accessibility (‘combo-centric’, cluster 2), (3) promoter accessibility alone (‘promoter-centric’, cluster 3), or (4) distal cis-regulatory accessibility alone (‘enhancer-centric’, cluster 4). In general, genes falling into clusters 2-4 exhibited larger fold-changes in expression between articular and growth plate chondrocytes compared to genes falling into the ‘unexplained variance’ category (cluster 1; FIG. 13A). Likewise, a greater proportion of genes from clusters 2-4 (genes whose variance in expression can be attributed to promoter or enhancer accessibility or both) were differentially expressed, compared to those from cluster 1 (whose variance cannot be attributed to differentially accessibility in any putative regulatory elements, FIG. 13A). Further analyses confirmed that sets of genes segregated with this method show increased sharing of direction (i.e., lineage bias) for the expected parameters (e.g., ‘combo-centric’ gene expression had a greater correspondence with cis-regulatory bias metric than did ‘promoter-centric’ gene expression).

Further analyses was conducted to characterize the TF motif enrichment in the DARs of genes belonging in clusters 2-4 (i.e., combo-centric, promoter-centric, and enhancer-centric genes) with the following initial restrictions: (1) motifs were only considered for TFs that were differentially expressed between hESC-derived articular and growth plate chondrocytes (TGFβ=124, BMP=83); enrichment of each motif was only considered for DARs, or promoters, in which the direction of accessibility (growth plate vs. articular) matched the direction of expression (growth plate vs. articular). For each motif demonstrating enrichment according to these criteria, further validation was done to determine whether enrichment could be significantly detected in the set of DARs/promoters for which the direction of accessibility was opposite to the direction of expression. This approach yielded a small number of motifs enriched in either promoter or enhancer sequences from cluster 2-4 genes, and that were biased towards articular chondrocytes or growth plate chondrocytes.

TABLE 5
Differentially expressed Transcription Factors (TFs) between
hESC-derived articular and growth plate chondrocytes

| Transcription factors upregulated | | Transcription factors upregulated in | | |
|---|---|---|---|---|
| in growth-plate chondrocytes | | articular chondrocytes | | |
| SOX8 | TBX3 | ETV1 | EGR1 | STAT6 |
| DLX6 | HEY2 | TEAD3 | NFYB | THAP11 |
| RUNX3 | MYC | POU2F2 | TWIST1 | SIX2 |
| HLTF | FOXF2 | BARX2 | EGR2 | FOXA3 |
| RFX3 | ZSCAN29 | MSANTD3 | BATF3 | ATF7 |
| MEF2C | GATA6 | MEF2A | IRF1 | ZNF692 |
| NFE2L1 | OSR1 | TFE3 | CENPB | KLF17 |
| YY1 | NR1I2 | MNT | NR1D1 | HINFP |
| KLF5 | ZNF711 | TCF3 | IRF3 | RELA |
| NFAT5 | HOXB13 | GLI2 | GLIS2 | ZNF449 |
| DLX5 | ISL2 | TEAD2 | ZNF384 | ZIC4 |
| PITX3 | ZBTB7B | NFKB2 | SIX1 | ZNF519 |
| HOXB6 | GFI1 | RFX2 | FOXP2 | FOXC2 |
| NKX3-2 | EN1 | MZF1 | SOX15 | HIC1 |
| E2F3 | MSX1 | XBP1 | NFATC1 | TGIF1 |
| TBX18 | HAND2 | HIF1A | BARX1 | SOX12 |
| NR3C1 | EBF1 | NFATC4 | RFX1 | ZNF354C |
| STAT1 | TBX20 | MYBL2 | BHLHE40 | MSC |
| DLX2 | HEY1 | NFATC2 | VEZF1 | ZBTB7A |
| VAX2 | OSR2 | E2F1 | KLF4 | ZBTB18 |
| PRRX1 | ZNF528 | HSF4 | LMX1B | LBX2 |
| MEF2D | ZEB2 | RELB | POU2F3 | ZNF467 |
| TFAP2E | EMX2 | DBP | LEF1 | POU6F1 |
| IRF6 | ZNF274 | ERF | ETV6 | ZBTB7C |
| ATF6 | CEBPB | MEOX2 | ZIC5 | MAFF |
| CREB1 | ZNF24 | GLI3 | TBX19 | NR2F2 |
| ONECUT2 | ESRRA | HLF | CREB3L4 | PBX1 |
| MXI1 | GLIS1 | NFKB1 | HES6 | MYBL1 |
| HOXB8 | ZFP42 | ELF2 | CREB5 | TCF4 |
| HOXB5 | GATA2 | PRDM4 | NFIB | ZNF136 |
| ELF1 | EGR3 | ELK3 | ZEB1 | ZNF682 |
| TBX2 | MAFA | FOXM1 | TBX6 | TEAD4 |
| SNAI1 | FOXO4 | PPARD | FLI1 | ZBTB14 |
| RREB1 | POU3F1 | BACH2 | TCF7L1 | MAFK |
| RUNX2 | TEAD1 | TFEB | KLF10 | POU3F3 |
| SOX9 | ZNF675 | BCL6 | ERG | FOXO6 |
| FOXA2 | ZNF460 | HES1 | CREB3L1 | PBX2 |
| ELK1 | SREBF2 | FOXP1 | RUNX1 | PBX2 |
| KLF2 | SOX18 | NFE2L2 | ZNF382 | PBX2 |
| PPARG | ZBED1 | ETV3 | EN2 | ETV5 |
| GRHL1 | HOXA10 | PROX1 | PKNOX2 | |
| CLOCK | | MSX2 | E2F7 | |

Analyses was conducted focusing on motifs most highly enriched in DARs from enhancer-centric (cluster 4) and combo-centric (cluster 2) genes, as these groups exhibit the strongest trends in differential expression across lineages (FIG. 13A). TF whose motifs were specifically enriched in TGFβ-treated articular cartilage-specific DARs included ETV1, FLI1, RELA, RFX2, NFKB1, RFX1, and ATF7, and significantly depleted or not significantly enriched for in BMP-specific growth plate cartilage-specific DARs (FIGS. 13B-13D). For example. RELA was one TF whose motifs were specifically enriched in TGFβ-treated articular cartilage-specific DARs (FIGS. 13B-13D), and significantly depleted in BMP-specific growth plate cartilage-specific DARs. TF whose motifs were specifically enriched in BMP-specific growth plate cartilage-specific DARs, and not significantly enriched in any TGFB-specific articular cartilage DARs, included FOXF2, FOXO4, FOXA2, CEBPB, RUNX2, DLX5 and EMX2 (FIGS. 13E-13G). RUNX2 motifs were specifically enriched in BMP-specific DARs (FIGS. 13E-13G), and not significantly enriched in any TGFB-specific articular cartilage DARs. DARs containing motifs for RELA or RUNX2 were specifically enriched for RELA or RUNX2 binding sites, respectively (see Methods). RELA, also known as p65, belongs to the NF-κB family of transcription factors that share a REL homology domain and can form transcriptionally active dimers with other family members. It is a transcriptional activator of SOX9, a master regulator of chondrocyte differentiation, as well as early differentiation and anabolic factors such as SOX6 and COL2A1, late-stage factor HIF-2a, and the catabolic gene ADAMTS5. It also plays a role in cartilage homeostasis and degradation in osteoarthritis. RUNX2, also known as CBFA1, PEBP2, or AML3, belongs to a class of transcription factors containing a Runt-homology domain (PMID: 8341710). RUNX2 has long been recognized as a ‘master’ skeletogenic factor, sitting atop a regulatory cascade governing osteoblast differentiation (PMID: 9182763; 9182762; 9182764). Since its initial discovery, the role of RUNX2 in skeletogenesis has expanded to include the regulation of chondrocyte hypertrophy in growth plate cartilage (PMID: 10213384; 10072783; 15107406). It also has a similar, though pathogenic, role in articular chondrocytes, which acquire hallmarks of hypertrophy in joint diseases such as osteoarthritis (PMID: 32913706; 31189030; 28539595).

Analyses were conducted on selected enhancer and promoter elements assigned to DEGs that have putative binding sites for RUNX2 or RELA in the hESC-derived chondrocytes to validate using CHIP-qPCR (Tables 6 and 7). To identify candidates for validation, putative binding sites were cross-referenced with ATAC-seq data collected from E15.5 mouse Col2a1+ and Col10a1+ chondrocytes, and published ChIP-seq data for several cell types. Targets that satisfied some or all of these criteria were selected, along with some genes that have been previously described in chondrocyte biology, and others only with binding sites that have overlapping ChIP-seq peaks in other cell types.

Seven putative RELA target loci (Table 6) were selected for confirmation by ChIP-qPCR, including several genes known to be involved in articular cartilage identity and maintenance. These include PRG4 (lubricin), a functional marker for the superficial zone of articular cartilage; LOXL2 (lysyl oxidase-like 2), which induces anabolic gene expression and plays a potential protective role against osteoarthritis (Alshenibr et al., Arthritis Res Ther., 2017, 19(1):179; PMID: 28764769); DKK3 (Dickkopf-3), a noncanonical member of the Dkk family of Wnt antagonists that plays a role in articular cartilage maintenance (PMID: 26687825); and TLR2 (Toll-like receptor 2), which mediates articular cartilage homeostasis (PMID: 24237425).

TABLE 6
Summary of candidate RELA targets

| | | | | Overlap with | Overlap with | Validated | Fold |
|---|---|---|---|---|---|---|---|
| Putative | | Distance from | | Mouse Col2a1+ | mouse Col10a1+ | ChIP-seq hits | enrichment in |
| Target | Chromosome location (hg19) | TSS of gene | | peaks | peaks | from other studies | ChIP-qPCR |
| PRG4 | chr1: 186201240-186201490 | −64.4 | kb | Y | — | 1 | 8.26 |
| LOXL2 | chr8: 23268990-23269240 | −7520 | bp | | — | 44 | 6.03 |
| LTBP2 | chr14: 75083374-75083624 | −4380 | bp | | — | 33 | 5.71 |
| GLIPR2 | chr9: 36135932-36137932 | +10 | bp | Y | — | 17 | 5.57 |
| DKK3 | chr11: 12101707-12101957 | −71.1 | kb | | — | 134 | 11.56 |
| TLR2 | chr4: 154577179-154577429 | +27.2 | kb | Y | — | 36 | 4.85 |
| COL15A1 | chr9: 101733568-101733818 | +26.6 | kb | Y | — | 1 | 1.28 |

TABLE 7
Summary of candidate RUNX2 targets

| | | | | Overlap with | Overlap with | Validated | Fold |
|---|---|---|---|---|---|---|---|
| Putative | | Distance from | | mouse col2+ | mouse col10+ | ChIP-seq hits | enrichment in |
| Target | Chromosome location (hg19) | TSS of gene | | peaks | peaks | from other studies | ChIP-qPCR |
| ACAN | chr15: 89312870-89313120 | −33.0 | kb | Y | | | 3.98 |
| ATOH8 | chr2: 85969150-85969400 | −11.9 | kb | Y | | 2 | 4.79 |
| C16orf72 | chr16: 9166745-9166995 | −18.5 | kb | | | 4 | 12.88 |
| COL10A1 | chr6: 116439814-116440064 | +7110 | bp | Y | Y | 4 | 2.44 |
| RCL1 | chr9: 4837930-4838180 | +44.8 | kb | | | 3 | 14.73 |
| WNT10B | chr12: 49366141-49368141 | −899 | bp | | | 4 | 5.96 |
| GPR153 | chr1: 6319685-6321685 | +699 | bp | Y | Y | 3 | 4.06 |
| MAP4K3 | chr2: 39719320-39719570 | −54.6 | kb | Y | Y | 1 | 11.36 |
| RXRA | chr9: 137178491-137178741 | −39.3 | kb | | | 4 | 6.42 |
| SCUBE1 | chr22: 43701886-43702136 | −36.8 | kb | Y | Y | 1 | 9.27 |

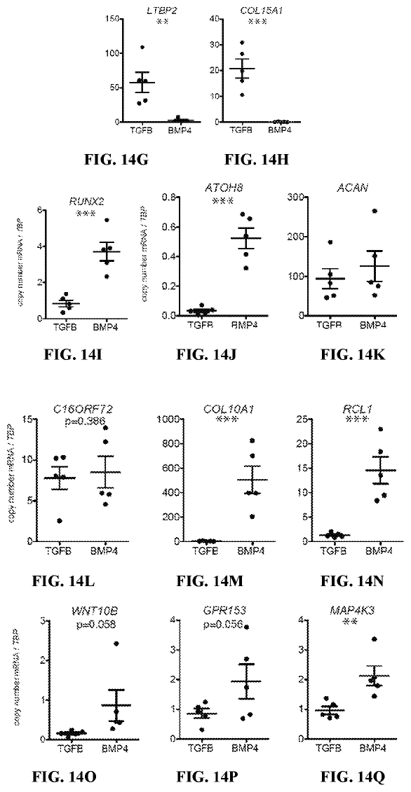

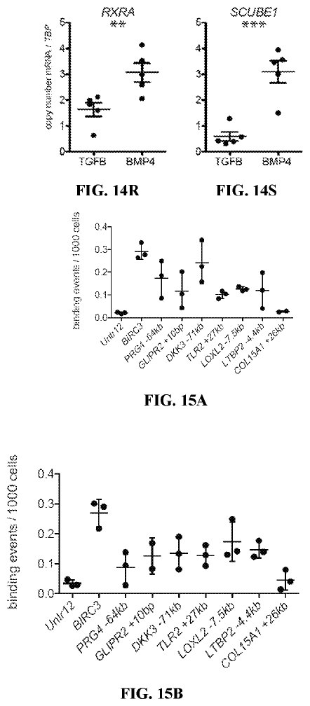

Also selected for validation were targets that have not been previously described in articular cartilage, including LTBP2, COL15A1, and GLIPR2. Representative binding regions with RELA motifs are the GLIPR2 promoter (overlapping with RELA ChIP-seq data and overlap with Col2a1+mouse chondrocytes; data not shown) and an upstream enhancer of LOXL2 (overlapping with RELA ChIP-seq data and histone acetylation peaks, data not shown). RELA and these putative target genes are expressed at significantly higher levels in hESC-derived articular cartilage (FIGS. 14A-14H). RELA, COL15A1, and LOXL2 were not differentially expressed between fetal epiphyseal and growth plate chondrocytes, however the remaining RELA targets were significantly higher in the fetal epiphyseal chondrocytes (Table 6 and data not shown). Notably, while COL15A1 is not a DEG in the fetal chondrocytes, its protein expression appears higher in the matrix of the fetal epiphysis compared to the matrix of the fetal growth plate.

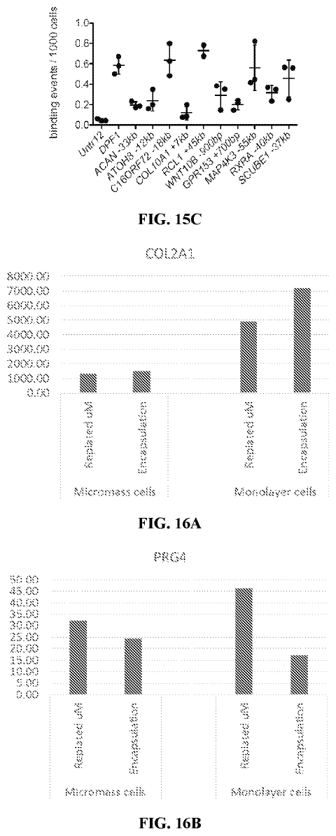

Following ChIP-mediated pulldown of genomic regions bound by RELA in TGFB-treated articular cartilage, six of the seven loci were enriched at least 2-fold compared to the negative control in the TGFB-treated articular cartilage (Table 6 and FIGS. 15A-15B), and five of the seven loci were enriched at least 5-fold. The binding region for the COL15A1 locus was only 1.3-fold enriched compared to the negative control in hESC-derived TGFB-treated articular cartilage, which suggests that RELA was not sufficiently bound to this locus in this sample or that COL15A1-expressing cells were not of sufficient abundance in these samples.

Ten putative RUNX2 targets (Table 7) were chosen for confirmation by ChIP-qPCR, including genes known to be important for chondrocyte and growth plate biology, including ACAN (Aggrecan), an essential proteoglycan in the extracellular matrix of both articular and growth plate cartilage (PMID: 28804204, PMID: 25446537); COL10A1 (Type X collagen), a marker of hypertrophic chondrocytes important for endochondral bone formation (PMID: 25321476); WNT10B, a Wnt family ligand thought to play a role in terminal chondrocyte differentiation and osteoblastogenesis (PMID: 17337262, PMID: 15728361); ATOH8 (Atonal homolog 8), a transcription factor important for chondrocyte proliferation and differentiation in the cartilaginous elements of endochondral bone (PMID:31449527); and RXRA (Retinoid X receptor alpha), a retinoic acid receptor that plays a role in endochondral ossification (Sun et al., 2019, Osteoarthritis and Cartilage, 27(1): S177-S178; PMID: 11277079). Also analyzed were targets previously undescribed in cartilage biology, including C16ORF72, RCL1, GPR153, MAP4K3, and SCUBE1 based on previously describe ChIP-seq interactions or homology with ATAC-seq peaks from mouse chondrocytes (Table 7). Representative gene regulatory elements with RUNX2 motifs are an upstream ATOH enhancer (overlapping with RUNX2 ChIP-seq data, Col2a1+ mouse chondrocytes, and histone acetylation marks, data not shown) and an upstream enhancer of ACAN (data not shown), which overlaps with peaks found in mouse Col2a1+ chondrocytes and is homologous to an enhancer identified in mouse chondrocytes (PMID: 29343853). RUNX2 and the putative target DEGs are more highly expressed in hESC-derived growth plate cartilage (FIGS. 14I-14S), with the exception of ACAN which is expressed in both cartilage lineages. Similarly, RUNX2 and most putative target genes were more highly expressed in fetal growth plate chondrocytes compared to fetal epiphyseal chondrocytes, with the exception of C16ORF72 which was expressed at a similar level. Following ChIP-mediated pulldown of genomic regions bound by RUNX2 in BMP-treated growth plate cartilage, all 10 target loci chosen for validation were enriched at least 2-fold compared to the negative control (Table 7 and FIG. 15C). Seven of the ten loci were enriched at least 5-fold compared to the negative control, confirming RUNX2 binding events at these gene regulatory elements.

As the great majority of putative DARs predicted as harboring motifs recognized by RELA and RUNX2 in hESC-derived articular and hESC-derived growth plate cartilage, respectively were indeed enriched compared to the negative control loci, these datasets are valuable for further exploration of the molecular mechanisms underlying chondrocyte fate decisions.

### Discussion

A thorough characterization of both the transcriptomic signatures and gene regulatory landscapes of human articular and growth plate chondrocytes is provided here. It also provides evidence that these hESC-derived lineages are molecularly similar to their in vivo counterparts, through transcriptomic profiling of human fetal epiphyseal and growth plate chondrocytes, and epigenetic profiling of mouse embryonic chondrocytes that were isolated from either Col2a1-(representing the majority of all mouse chondrocytes) or Col10a1-reporter mice (representing mouse growth plate chondrocytes). Specifically, we found strong correlations between hESC-derived articular cartilage and fetal epiphyseal samples, and likewise between hESC-derived growth plate cartilage and fetal growth plate samples.

We performed extensive experimental validation of differentially expressed genes, confirming lineage-specific patterns across multiple independent hESC differentiation experiments and primary cell datasets. Receptor-ligand pairs, Fibroblast growth factor 18 (FGF18) and its receptor FGFR3 (PMIDs: 31290205, 12960068, 23060229, 15781473), and Parathyroid hormone-like hormone (PTHLH) and its receptor PTH1R ((PMID: 27142453, PMID: 8314082), are known to be differentially expressed between articular and growth plate cartilage, respectively. We found these expression patterns, and those of other known markers, to hold true in both the hESC-derived and fetal chondrocytes. In addition to known markers, we identified novel genes that mark distinct cartilage lineages, such as MEOX1 and CHI3L1 in the articular cartilage lineage, and EFHD1 in the growth plate cartilage lineage. MEOX1 and CHI3L1, whose expression has been reported in the axial skeleton and in osteoarthritic cartilage respectively, had not yet been identified in developing articular cartilage. EFHD1 was found to be strongly localized to hypertrophic cells in hESC-derived and fetal growth plate chondrocytes. Previously studied in its role as a calcium sensor (PMID: 26975899), EFHD1 could play a role in mediating cellular response to calcium in hypertrophic chondrocytes (PMID: 11404353). We also surprisingly found tenonodulin (TNMD) to be expressed in the superficial zone of articular cartilage, co-expressed with PRG4. TNMD, closely related to chondromodulin 1 (CNMD), is known as a functional marker for tenocytes (PMID: 15632070). While there is conflicting evidence of TNMD expression in resting and proliferating chondrocytes of the growth plate cartilage (PMID: 11357195, 18239943), TNMD expression in the superficial zone of articular cartilage has not been previously described. Although this result was seemingly unexpected, both cartilage and tendons/ligaments rely on TGFB signaling, and they likely arise from a common developmental progenitor (PMID: 19304887, PMID: 18295755, PMID: 27292641). This interesting finding warrants further exploration of the developmental relationship between cartilage and the adjacent connective tissues in the joint. Further, these data uncover several other novel genes as yet unstudied in chondrocyte biology, underscoring the potential utility of tissue- or zone-specific markers, and the opportunity to investigate their function(s) in cartilage development or maintenance in human cells and other models.

Despite this strong overall correspondence between hESC-derived and fetal cartilages, there were some notable exceptions, including genes whose expression patterns were opposite those seen in vitro. This was an expected result, as the cartilage dissected from fetal samples are more heterogeneous than the hESC-derived tissues, and there is overlap the chondrocyte cells in these samples due to the continuous structure of fetal cartilage and the approximate dissection boundary. For example, the dissected epiphyseal cartilage includes perichondrium, resting zone chondrocytes, proliferative chondrocytes, in addition to chondrocytes that will participate in events related to the secondary ossification center and those that will eventually give rise to the neonatal and adult articular cartilage. Likewise, the growth plate cartilage includes proliferative, pre-hypertrophic, and hypertrophic chondrocytes, in addition to perichondrium cells, although our micro-dissection approach was to omit osteoblasts and hematopoietic cells. Furthermore, it is yet unclear where hESC-derived articular and growth plate cartilage reflect developmental time relative to fetal cartilage. Some obvious differences in developmental stage are the presence of late stage growth plate marker gene expression such as Integrin Binding Sialoprotein (IBSP) hESC-derived growth plate chondrocytes that was lacking in the fetal growth plate chondrocytes, and the presence of a distinct superficial zone of cartilage in the hESC-derived articular cartilage that is less developed in the fetal tissue used in this study (i.e., we indicated that the surface of the epiphysis corresponded to the site of the future superficial zone of articular cartilage). The potentially more developed superficial zone in the hESC-derived articular cartilage may be why superficial-zone-specific genes, such as COL15A1, a non-fibrillar basement membrane-associated collagen (PMID: 24043668), were identified as differentially expressed in the hESC-derived articular cartilage but not in the fetal epiphyseal cartilage. Future studies focused on transcriptomic profiling chondrocytes at the single-cell level and/or over differentiation and time will address some of these standing questions.

Given our confidence in the divergent properties of the hESC-derived chondrogenic lineages, and that they reflect true in vivo lineage properties, we also sought to use this system to define putative gene-regulatory networks (GRNs) which may govern lineage specification and gene expression patterns in developing chondrocytes. We used ATAC-seq to probe the regulatory landscape of hESC-derived chondrocytes, as this approach offers insight into the potential binding of all factors using only a single assay. By generating paired ATAC- and RNA-seq libraries on hESC-derived articular (TGFB-treated) and growth plate (BMP-treated) chondrocyte lineages, we were able to consider chromatin accessibility and nearby gene expression at a locus-specific level for key lineage-marker genes as well as more broadly across the genome. Additionally, this assay also pointed us to potential targets of the candidate regulatory transcription factors (TFs).

As TFs typically have key roles in governing lineage-specific expression patterns, we identified a number of factors which demonstrate biases in expression across lineages, and for which motif occurrence is enriched in putative lineage-biased regulatory elements. Finding that a simple model of a GRN was insufficient to explain the behaviors observed in our epigenetic and expression datasets, we applied a per-locus approach to integrating our ATAC- and RNA-seq genes, defining sets of genes with different putative regulatory behaviors. We found that these groups exhibited different patterns of differential gene expression, associations with chromatin accessibility data, and, importantly, enriched occurrence of lineage-biased TFs (FIG. 4). Notably, our finding that grouped genes differed in their degree of differential expression is consistent with a previous study which stratified immune genes on the basis of regulatory behaviors. We leveraged these findings to identify TFs exhibiting enrichments for DARs around DEGs we defined as either ‘enhancer-centric’ or ‘combo-centric’-enrichments exclusive to a particular lineage (FIG. 5). Through this analysis, we identified RELA and RUNX2 as transcription factors, among others, which may play a role in lineage-specific regulatory networks, and used the results of our integrative ATAC-/RNA-seq analysis to prioritize candidate regulatory regions which they may bind to regulate the differential expression of lineage-specific genes. Remarkably, when we performed ChIP-qPCR against these candidate regions, we found that a majority (16 of the 17 tested) did in fact bind the predicted TF in the expected lineage. These findings emphasize the co-use of the epigenetic and expression datasets generated in this study in defining putative gene regulatory networks which may be active in developing chondrocyte populations, and in identifying key regulatory factors controlling these networks.

Our transcriptomic profiling approach uncovered several interesting TFs in both hESC-derived cartilage lineages, including those that had not been identified as significantly differentially expressed in the smaller subset of samples in which we also performed ATAC-seq. Many of these TFs and their associated family members showed similar expression patterns in the fetal cartilage specimens, and several have been previously identified in the context of cartilage and joint biology, validating our data and the hESC-model system we've studied here. Such TF families identified in the TGFB-induced hESC-derived articular cartilage include the ETS factors, containing a conserved ETS DNA-binding domain, include the polyomavirus enhancer activator 3 (PEA3) family members (ETV1, ETV4, ETV5), and the ETS-related gene (ERG) family members (ERG, FLIT, FEV) (PMID: 23870508). Here, we specifically pinpointed ETV1 and FLI1 as regulators of enhancer- and combo-centric genes in the hESC-derived articular cartilage lineage (FIG. 5). PEA3 family members are significantly differentially expressed in both hESC-derived articular cartilage and in fetal epiphyseal chondrocytes. They are FGF-responsive genes and there is some evidence that loss of these proteins results in reduced and disorganized brachial cartilage (PMID: 26555052). ERG and FLIT are differentially expressed in hESC-derived articular cartilage (but not significant in fetal data), while FEV is differentially expressed in growth plate cartilage (not in fetal). ERG has been well-studied for its role in long-term maintenance of articular cartilage, and, along with FLIT, upregulates articular cartilage genes such as PTHLH and PRG4 (PMID: 17336282, https://doi.org/10.1016/j.joca.2013.02.063). The CREB family of TFs include CREB5 and CREB3L1, both of which are differentially expressed in hESC-derived articular cartilage (CREB5 is differentially expressed in fetal epiphyseal cartilage, but CREB3L1 is not significant in fetal data). CREB5 is a known regulator of PRG4 expression in the articular cartilage (PMID: 33712729), and shares sequence homology with the ATF family of TFs, such as ATF7 we highlight as a regulator of combo-centric genes in the hESC-derived articular cartilage lineage (FIG. 5). Nuclear factor of activated T-cells (NFAT) family members NFATC2 and NFATC4 are both differentially expressed in hESC-derived articular cartilage (NFATC4 is also differentially expressed in fetal epipysis). NFATC2 is also more highly expressed in superficial zone chondrocytes compared to deep zone chondrocytes in bovine cartilage, and NFAT family members play a role in chondrocyte gene expression and articular cartilage maintenance (PMID: 24248346, PMID: 12239209, PMID: 24257415). The homeobox proteins MEOX1 and MEOX2 and the LIM-homeobox protein LHX9 were also DEGs in both hESC-derived articular cartilage and fetal epiphyseal chondrocytes. MEOX1 and MEOX2 are essential for the development of all somite compartments and for the normal development of the cranio-cervical joint (PMID: 19520072). LHX9 is induced by FGF-signaling and has been previously studied for its role in the progression of osteosarcomas (PMID: 31788020). These TFs and TF families, among others we identified in these studies, warrant further exploration for their individual and joint roles in articular cartilage development and stability.

In the growth plate lineage, members of the DLX family of TFs, DLX2, DLX5, DLX6, were highly expressed in growth plate cartilage (DLX5 and DLX6 also expressed in fetal growth plate, DLX2 only differentially expressed in hESC-derived growth plate), and are known to be critical regulators of cartilage differentiation during endochondral ossification (PMID: 17051482). In particular, DLX5 has been shown to regulate the differentiation of immature proliferating chondrocytes into hypertrophic chondrocytes, and in osteoblast differentiation (PMID: 12482714). Similarly, two RUNX family members, RUNX2 and RUNX3 are differentially expressed in both hESC-derived and fetal growth plate cartilage. RUNX2 is a critical TF for chondrogenic maturation and osteoblast differentiation, and works in concert with DLX5 and SP7 for the proper skeletal development (https://doi.org/10.1016/S1348-8643(14)00032-9). RUNX3 works redundantly with RUNX2 in chondrocyte maturation (PMID: 15107406). The forkhead box (FOX) proteins are a superfamily of TFs, of which several members are differentially expressed in either articular cartilage or growth plate lineages. Of this large family, FOXA2, expressed in the hESC-derived growth plate cartilage, is a critical regulator of hypertrophic differentiation in chondrocytes (PMID: 22595668) and has been implicated in cartilage degradation and OA progression (www.oarsijoumal.com/article/S1063-4584(19)30273-0/fulltext). Myocyte enhancer factor 2c (MEF2C) is differentially expressed in both hESC-derived and fetal growth plate cartilage, and activates the genetic program for hypertrophy during endochondral ossification (PMID: 17336904). These TFs, and others identified in the studies herein, can now be investigated for their biological role in growth plate biology and chondrocyte function.

The data here are valuable resources for studying human articular and growth plate cartilage development. Thus, it has been established and validated the in vitro human pluripotent stem cell cartilage differentiation system as a robust and useful tool in investigating articular and growth plate cartilage lineages. This is particularly important for understanding how to specify and maintain articular cartilage, since diseased and sometimes even regenerating tissue following cartilage damage display hypertrophy-like changes (PMID: 22178514). From tissue-specific transcriptomic data, we have identified several novel genes that mark the two different tissues, and have further identified and validated zone-specific markers of cartilage. The effort to identify genes and networks that regulate cartilage development can be propelled by this comprehensive study and analyses of transcriptomic and epigenetic signatures of articular and growth plate cartilage. We believe that this comparative perspective will prominently aid in our understanding of cartilage development and joint-disease biology.

### Example 10: Comparison of Micromasses Derived from High-Density Seeding Versus Low-Density Seeding (Monolayer) of Paraxial Mesoderm at Stage III

Macroscopic morphology of micromasses derived using different protocols was investigated after 1.5 weeks, 3 weeks, and 6 weeks (data not shown). High-density seeding of paraxial mesoderm at Stage III resulted in uneven and bumpy micromasses, and uneven toluidine blue staining, suggesting inefficient chondrogenesis. Micromasses derived from monolayer-derived chondrocytes were evenly stained with toluidine blue, suggesting uniform and efficient chondrogenesis. These data clearly indicate that monolayer derived micromasses are higher quality than those derived from high-density seeding of paraxial mesoderm at Stage III (images not shown).

3-week-old micromasses using high-density plating of paraxial mesoderm at Stage III cannot hold their shape when relocated to petri dish using forceps. On the contrary, 1.5-week-old monolayer-derived micromasses can hold their shape better than 3-week-old micromasses derived using high-density plating of paraxial mesoderm at Stage III. These observations clearly indicate structural differences in these micromasses derived from different methods. The micromass derived from low-density monolayer seeding of paraxial mesoderm at Stage III provide higher quality, more extracellular matrix, more uniform distribution of extracellular matrix (images not shown).

4-week-old micromass and 6-week-old micromass derived from high-density seeding of paraxial mesoderm at Stage III often have uneven morphology and areas with less dense matrix and less proteoglycan, indicating less than optimal chondrogenic efficiency. Monolayer-derived micromass after 4-weeks and 6 weeks display even morphology and uniform staining of the monolayer-derived micromass indicates highly efficient chondrogenesis (images not shown).

The superior cartilage-like quality of monolayer-derived cartilage tissues was further confirmed by the expression levels of COL2A1 (cartilage gene) after 6 weeks in micromass culture or 6 weeks after encapsulation in RAD16-I biomaterial (FIG. 16A). COL2A1 expression is higher in tissues derived from monolayer-derived cells compared to those derived from high density micromass culture of paraxial mesoderm cells (FIG. 16A). PRG4 expression (articular cartilage gene) was also expressed at similar levels in monolayer-derived micromasses and encapsulations compared to 6-week-old micromass or encapsulations derived using high-density seeding of paraxial mesoderm at Stage III (FIG. 16B). In general the levels of PRG4 are higher in micromass compared to encapsulations for both populations (FIG. 16B).

Gene expression graphs show monolayer derived micromasses undergo chondrogenesis earlier and more efficiently than original protocol micromasses. Gene expression was compared between 1 and 4 weeks of chondrogenic culture in micromass, monolayer culture, and monolayer derived micromasses to determine the comparative levels of chondrogenic genes COL2A1, SOX9, PRG4 and ACAN. Data suggests that monolayer culture and micromass culture initiated with monolayer-derived cells undergo chondrogenesis both earlier and more efficiently based on the upregulation of COL2A1, SOX9 and ACAN gene expression, and generate articular cartilage based on the upregulation of PRG4. FIG. 16C-F depict copy number mRNA in stage III paraxial mesoderm (day 14), micromass cultures derived from paraxial mesoderm plated into micromass at stage III (Micromass), monolayer cultures derived from paraxial mesoderm plated in monolayer at stage III (Monolayer), and Monolayer-derived micromass cultures derived from monolayer cells plated into micromass culture after 4 weeks, after 1 week, 2 weeks, 3 weeks, or 4 weeks, as indicated.

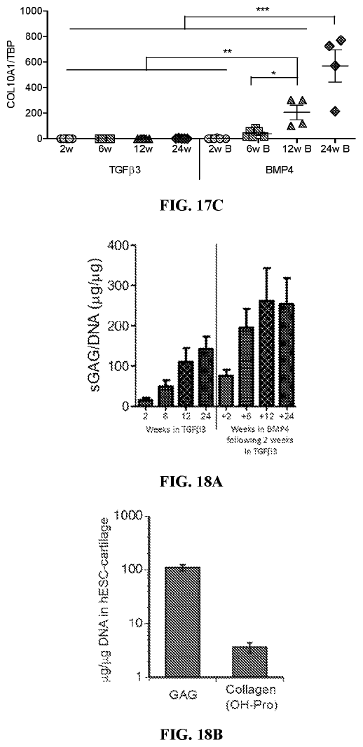

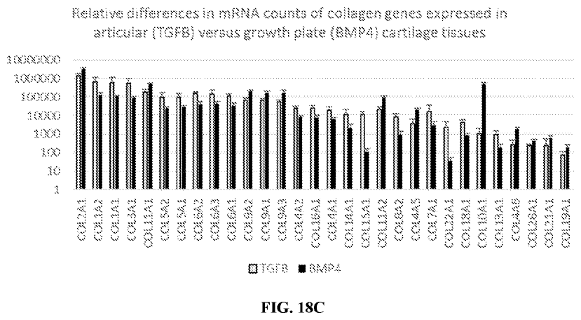

FIGS. 17A-C depict copy number COL10A1 mRNA normalized to TBP in micromass cultures at indicated timepoints and treatment regimens. TGFβ3-treated articular cartilage tissues were cultured in TGFβ3-supplemented media for indicated periods of time (2 weeks, 4 weeks, 6 weeks, 8 weeks, 10 weeks, or 12 weeks) until they were challenged with BMP4 by switching the media supplementation from TGFβ3 to BMP4 (e.g., BMP4 at ((@) 2 weeks indicates that cultures were treated with TGFβ3 for 2 weeks and then switched to BMP4 supplementation for an additional (+) 2, 6, 12 or 24 weeks). Micromass cultures that have been treated with TGFβ for 8 weeks or less prior to the BMP4-treatment regimen were able to respond to BMP4 and upregulate COL10A1 mRNA, indicating the cells remain bi-potent and can undergo growth plate cartilage differentiation. Micromass cultures that have been treated with TGFβ for at least 10 weeks prior to BMP4 treatment do not upregulate COL10A1 mRNA upon BMP4 challenge, and are thus resistant to BMP4-mediated growth plate differentiation. Data represents average of 4 biological replicates, error bars indicate standard error. FIG. 17A shows stability of hPSC-derived Articular Cartilage tissues is achieved after 8-10 weeks of TGFβ treatment as they become resistant to BMP4 challenge and growth plate chondrocyte differentiation. FIGS. 17B and 21C show cartilage lineages are stable long term in culture as PRG4 gene expression (FIG. 17B) remains significantly higher in the TGFβ-induced articular cartilage lineage, and COL10A1 expression (FIG. 17C) is exclusively expressed by BMP4-induced growth plate cartilage for up to 24 weeks. Distinct cartilage tissues do not express genes associated with the other lineage even after 24 weeks of culture in vitro.

FIG. 18A shows examples of quantified amount of sulfated (s) glycosaminoglycans (GAG) (μg per μg of DNA content) in micromass cultures cultured in the presence of TGFβ3 or BMP4 for indicated times (weeks). sGAG content increases over time in both cartilaginous tissues. FIG. 18B depicts representative quantification of both sulfated GAG and hydroxy-proline (OH-Pro; a surrogate biochemical quantification of collagen content), in TGFβ3-treated articular cartilage tissues cultured for 12 weeks. Values were calculated as μg per μg of DNA content per culture. Error bars represent standard error of the mean. FIG. 18C depicts the relative differential expression levels of representative collagen genes in articular (TGFβ) and growth plate (BMP) cartilage micromass tissues. Values represent mean (n=6) counts from RNA-sequencing, error bars indicate standard deviation. Graph depicts only those genes that were found to be significantly differentially expressed, i.e., non-inclusive of all collagen genes expressed in these tissues.

Collagen genes expressed higher in articular cartilage: COL6A3; COL4A4; COL14A1; COL22A1; COL16A1; COL8A2; COL8A1; COL12A1; COL4A2; COL5A2; COL24A1; COL3A1; COL1A2; COL18A1; COL4A1; COL1A1; COL5A1; COL6A2; COL6A1; COL7A1; COL15A1; COL13A1; COL25A1.

Collagen genes expressed higher in growth plate cartilage: COL20A1; COL10A1; COL11A1; COL4A6; COL4A5; COL11A2; COL2A1; COL9A1; COL9A2; COL9A3

Glycoproteins genes expressed higher in articular cartilage: SBSPON
THBS4; TNFAIP6; SPON1; SRPX; LAMA1; COCH; COMP; FN1; AEBP1; MXRA5; MATN2; TGFBI; SNED1; LAMB3; ECM1; EFEMP2; LAMA2; LAMB2; MFAP2; MFAP4; SRPX2; LTBP3; POSTN; CILP; PCOLCE; FBLN7; LTBP2; EMILIN1; VWA1; FBLN1; MGP; GAS6; CTHRC1; IGFBP7; FBLN5; FNDC1; ECM2; CRISPLD2; THBS3; LAMC3; HMCN1; LTBP4; CILP2; LTBP1; SVEP1; PXDN; THBS2; DPT; EDIL3; ABI3BP; IGFBP6; LAMC2; NELL1; MFAP5; LAMA3; SPARC; FBN2; FBN1; ELN; THBS1; EMILIN3; SMOC1; AGRN; NTN3; SSPO; OTOG;
Glycoproteins genes expressed higher in growth plate cartilage: NID1; SLIT3; NTNG1; LGI4; PAPLN; EMILIN2; RSPO2; NTNG2; RSPO4; MATN4; MATN3; NPNT; SPP1; DMP1; SPARCL1; NELL2; VWDE; VWA5A; TSKU; IGFBP2; FGL2; TINAGL1; EMID1; TNC; CRISPLD1; SPON2; IGSF10; GLDN; LAMA4; MFAP3; MFGE8; PCOLCE2;
Proteoglycan genes expressed higher in articular cartilage: LUM; FMOD; PODNL1; BGN; ASPN; HAPLN3; PRG4; PODN; OGN;
Proteogycan genes expressed higher in growth plate cartilage: IMPG2; CHAD; SPOCK3; CHADL; HAPLN1; DCN; HAPLN2; PRELP; ACAN;
Secreted factor genes expressed higher in articular cartilage: EGFL8; MEGF6; SFRP1; LEFTY2; WFIKKN1; AMH; FSTL1; BMP5; SCUBE3; GDF6; IL17D; PDGFC; GDF7; CHRDL1; GDF5; WIF1; NRTN; THPO; FGF2; ANGPTL6; INHBA; TGFβ3; VEGFB; TNFSF12; FGF18; ILI1; NGF; CLCF1; IL10; CX3CL1; VWC2; S100A6; FGF1; TNFSF10; PDGFA; WNT5B; ANGPTL1; ANGPTL7; SFRP5; EGFL6; ARTN; FGF9; CHRD; MEGF10; CBLN3; MST1; LIF; S100A3; S100A16; HBEGF; S100A4; TNFSF9; CRLF1; WNT9A; INHBE; CTF1; IGF1; SFRP2; NRG1; S100B; NTF3; FGF11; ANGPTL4; FSTL3; TGFβ1; VEGFC; HCFC1; S100A2;
Secreted factor genes expressed higher in growth plate cartilage: TGFA; WNT16; W NT11; CXCL13; KITLG; WNT5A; BMP8B; MEGF11; ANGPTL5; WNT10B; ANGPTL2; S100A13; WNT3; WFIKKN2; S100P; ANGPT1; S100A1; SCUBE1; BMP4; HGF; NRG4; IHH; FGFBP2; FGF7; ISM1; EGF; BMP2; IL17B; CXCL14; FGF14; GDF15; FGF13; WNT2B; PDGFD; PIK3IP1; CHRDL2; FST; WNT4; CRHBP; S100Z; BMP6; HHIP; TGFβ2; SCUBE2; ANGPT2; FRZB; MEGF9; CSF1;
ECM-affiliated genes expressed higher in articular cartilage: ANXA1; PLXDC2; MUC1; PLXDC1; LGALS1; MUC20; ANXA6; COLEC12; PLXNA4; C1QTNF5; SEMA3C; SEMA3A; SEMA4C; CSPG5; C1QL1; GREM1; SEMA3F; SEMA4D; SDC1; C1QTNF2; CLEC11A; GPC2; SEMA4A; CLEC2D; SEMA6A; C1QTNF8; C1QL4; CLEC3A; C1QTNF1; ANXA3; SDC2;
ECM-affiliated genes expressed higher in growth plate cartilage: MUC5B; ANXA5; SEMA5A; LGALS3; SDC4; SEMA4G; PLXNA1; PLXNB3; SEMA3D; GPC1; GPC4; ANXA2; SEMA3E; CLEC3B; GPC6; C1QTNF7; SEMA5B; GPC5; SEMA6B; GPC3; PLXNB1; SDC3.

1. A method for chemically inducing differentiation of human pluripotent stem cells (hPSC) into chondrocytes comprising:
(a) inducing the formation of primitive streak mesoderm from hPSC in a first culture medium;
(b) inducing the formation of paraxial mesoderm from primitive streak mesoderm in a second culture medium; and
(c) inducing conversion of paraxial meabstractsoderm cells into chondrocytes in a third culture medium;
wherein the paraxial mesoderm cells are cultured in a cell medium at a low cell density.

2. The method of claim 1, wherein the first cell culture medium for culturing the hPSC is supplemented with one or more of FGF agonists, BMP4 agonists, and TGFβ agonists; and optionally a Wnt agonist, in effective amounts for an effective amount of time to generate PSM cells expressing the cell surface markers CD56 and PDGFRα.

3. The method of claim 2, wherein: (a) the FGF agonist is selected from the group consisting of FGF, bFGF, FGF2, FGF4, FGF9, FGF19, FGF21, FGF3, FGF5, FGF6, FGF8a, FGF16, FGF 17, FGF18, FGF20 and FGF23, optionally active conjugates and fragments thereof; (b) the BMP4 agonist is selected from the group consisting of BMP4, GDF5, GDF6, GDF7, BMP4, BMP2, BMP6, BMP7 and BMP10; (c) the TGFβ agonist is selected from the group consisting of TGFβ 1, TGFβ, TGFβ 3, and Activin A; (d) the Wnt agonist is selected from the group consisting of CHIR99021, SB216763, TWS119, CHIR98014, Tideglusib, SB415286, LY2090314, CHIR-98014, AZD1080, TDZD-8 and wnt3a; and/or the FGF agonist is FGF, optionally wherein the BMP4 agonist is BMP4, and optionally wherein the TGFβ agonist is Activin A.

4. (canceled)

5. (canceled)

6. (canceled)

7. (canceled)

8. The method of claim 1, wherein: (a) step (a) preferably results in the formation of embryoid bodies; (b) step (a) omits a step that results in the formation of embryoid bodies; (c) the second cell culture medium for culturing the primitive streak mesoderm cells induced in step (a) is supplemented with a BMP4 inhibitor and/or an FGF agonist; and optionally a Wnt antagonist and/or a TGFβ inhibitor, in effective amounts for an effective amount of time to generate formation of paraxial mesoderm cells expressing D73, CD105 and/or PDGFRβ; and/or the FGF agonist is selected from the group consisting of FGF, bFGF, FGF2, FGF4, FGF9, FGF19, FGF21, FGF3, FGF5, FGF6, FGF8a, FGF16, FGF 17, FGF18, FGF20 and FGF23, optionally active conjugates and fragments thereof.

9. (canceled)

10. (canceled)

11. The method of claim 8, wherein the BMP4 inhibitor is selected from the group consisting of Dorsomorphin, and LDN 193189 dihydrochloride.

12. (canceled)

13. The method of claim 8, wherein the Wnt antagonist is selected from the group consisting of IWP2 (N-(6-Methyl-2-benzothiazolyl)-2-[(3,4,6,7-tetrahydro-4-oxo-3-phenylthieno[3,2-d]pyrimidin-2-yl)thio]-acetamide); Dickkopf-related protein 1 (DKK1), Wnt-C59 (4-(2-Methyl-4-pyridinyl)-N-[4-(3-pyridinyl)phenyl]benzeneacetamide) and XAV939 (3,5,7,8-Tetrahydro-2-[4-(trifluoromethyl)phenyl]-4H-thiopyrano[4,3-d]pyrimidin-4-one), and/or wherein the TGFβ inhibitor is selected from the group consisting of SB431542, GW788388, and A-83-01.

14. (canceled)

15. The method of claim 1, wherein: (ii) the third cell culture medium for culturing the paraxial mesoderm cells induced in step (b) is supplemented with effective amounts of a combination of agents selected from the group consisting of a TGFβ agonist, an FGF agonist, and a cyclic AMP agonist, for an effective amount of time to produce chondrocyte cells expressing one or more of SOX9, COL2A1, and PRG4; (ii) the paraxial mesoderm cells in step (b) are cultured at a low cell density between about 20×10³ per cm² and 20×10⁴ per cm2, inclusive; (iii) the number of the chondrocyte cells expressing one or more of SOX9, COL2A1, and PRG4 is about 10%, 20%, 30%, 40%, 50%, 60%, 70%, 80%, 90%, or over 100% more when derived from the paraxial mesoderm cells in step (b) cultured at a low cell density than if the paraxial mesoderm cells in step (b) are cultured at a high cell density provided having the same number of a starting population of paraxial mesoderm cells and/or (iv) the method further comprising the step of (d) culturing the chondrocytes in high density micromass or encapsulating the chondrocytes within biomaterials to produce mature chondrocytes and/or a cartilage tissue thereof in cell culture media containing a TGFβ agonist.

16. The method of claim 15, wherein: (a) the TGFβ agonist is selected from the group consisting of TGFβ1, TGFβ, TGFβ3, and Activin A; (b) the FGF agonist is selected from the group consisting of FGF, bFGF, FGF2, FGF4, FGF9, FGF19, FGF21, FGF3, FGF5, FGF6, FGF8a, FGF16, FGF 17, FGF18, FGF20 and FGF23, optionally active conjugates and fragments thereof; and/or (c) the cyclic AMP agonist is selected from the group consisting of prostaglandin E2, dbcAMP, 8-bromo-cAMP, genistein, forskolin, colforsin, and rolipram.

17. (canceled)

18. (canceled)

19. (canceled)

20. (canceled)

21. (canceled)

22. The method of claim 15, wherein: (a) the TGFβ agonist is selected from the group consisting of TGFβ1, TGFβ, TGFβ3, and Activin A; (b) the chondrocytes are encapsulated in hydrogels as tissue engineering scaffolds; (c) the method further comprising the step of freezing the chondrocytes, or the cartilage tissue thereof, in a salt solution and one or more cryoprotectants, optionally, wherein the method further comprising the step of thawing the frozen chondrocytes, or the cartilage tissue thereof, for further culture and/or tissue implants in vivo.

23. (canceled)

24. (canceled)

25. (canceled)

26. A chondrocyte or a cartilage tissue thereof prepared according to the method of claim 1.

27. (canceled)

28. A method of treating or preventing one or more disease or disorders needing cartilage repair in a subject in need thereof, comprising implanting the chondrocyte or the cartilage tissue thereof of claim 26.

29. The method of claim 28, wherein the subject has osteoarthritis, osteochondritis dissecans, polychondritis, other chondropathies, or injuries or damages affecting the cartilage, wherein one or more symptoms associated with osteoarthritis, osteochondritis dissecans, polychondritis, other chondropathies, or injuries or damages affecting the cartilage, is treated or ameliorated.

30. (canceled)

31. A tissue culture medium for inducing conversion of paraxial mesoderm cells into chondrocytes, comprising a combination of agents selected from the group consisting of a TGFβ agonist, an FGF agonist, and a cyclic AMP agonist.

32. The tissue culture medium of claim 31, in effective amounts to produce chondrocyte cells expressing one or more of SOX9, COL2A1, and PRG4.

33. The tissue culture medium of claim 31, wherein: (a) the TGFβ agonist is selected from the group consisting of TGFβ1, TGFβ, TGFβ3, and Activin A; (b) the FGF agonist is selected from the group consisting of FGF, bFGF, FGF2, FGF4, FGF9, FGF19, FGF21, FGF3, FGF5, FGF6, FGF8a, FGF16, FGF 17, FGF18, FGF20 and FGF23, optionally active conjugates and fragments thereof; (c) the cyclic AMP agonist is selected from the group consisting of prostaglandin E2, dbcAMP, 8-bromo-cAMP, genistein, Forskolin, colforsin, and rolipram.

34. The tissue culture medium of claim 31, wherein: (a) the TGFβ agonist is TGFβ3, at a concentration between about 1 ng/ml and about 50 ng/ml, inclusive; (b) the FGF agonist is bFGF, at a concentration of between about 1 ng/ml and 100 ng/ml, inclusive; and/or the cyclic AMP agonist is Forskolin, at a concentration between about 5 μM and about 100 μM, inclusive.

35. The tissue culture medium of claim 34, wherein: (a) the tissue culture medium comprises TGFβ agonist, wherein the concentration of the TGFβ agonist is between about 5 ng/ml and about 20 ng/ml, inclusive; or preferably about 10 ng/ml; (b) the tissue culture medium comprise an FGF agonist, wherein the concentration of the FGF agonist is between about 5 ng/ml and 50 ng/ml, inclusive; or preferably about 10 ng/ml; and/or (c) the tissue culture medium comprise a cyclic AMP agonist, wherein the concentration of the cyclic AMP agonist is between about 10 μM and about 50 μM, inclusive; or optionally about 30 μM.

36. (canceled)

37. (canceled)

38. (canceled)

39. (canceled)

40. (canceled)

41. (canceled)

42. The tissue culture medium of claim 31, wherein the combination of agents are effective in inducing a higher number of the chondrocyte cells expressing one or more of SOX9, COL2A1, and PRG4 is about 10%, 20%, 30%, 40%, 50%, 60%, 70%, 80%, 90%, or over 100% more when derived from the paraxial mesoderm cells cultured at a low cell density than if the paraxial mesoderm cells are cultured at a high cell density provided having the same number of paraxial mesoderm cells as a starting population.

43. The tissue culture medium of claim 42, wherein the paraxial mesoderm cells are cultured at a low cell density between about 20×10³ per cm² and 20×10⁴ per cm², inclusive.
